SEQUENCE LISTING (166 sequences)

SEQ ID NO: 1 - protein, 98 aa, Artificial Sequence
  Synthetic Antibody
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAA

SEQ ID NO: 2 - protein, 15 aa, Artificial Sequence
  Linker sequence
GGGGSGGGGSGGGGS

SEQ ID NO: 3 - protein, 123 aa, Artificial Sequence
  Synthetic Antibody
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAAERPTSDGSTGGMDVWGQGTLVTVSS

SEQ ID NO: 4 - protein, 106 aa, Artificial Sequence
  Synthetic Antibody
DIQMTQSPSSLSASVGDRVTITCRASQSISSYLNWYQQKPGKAPKLLIYGASTLQSGVPSRFSGSGSGTDFTLTISSLQPEDFATYYCQQSYSSGTFGQGTKVEIK

SEQ ID NO: 5 - protein, 124 aa, Artificial Sequence
  Synthetic Antiboy
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAAEQWGTNDGPDGGMDYWGQGTLVTVSS

SEQ ID NO: 6 - protein, 107 aa, Artificial Sequence
  Artificial Antibody
DIQMTQSPSSLSASVGDRVTITCRASQTIGSHLNWYQQKPGKAPKLLIYGTSNLQSGVPSRFSGSGSGTDFTLTISSLQPEDFATYYCQQSYSSPLTFGQGTKVEIK

SEQ ID NO: 7 - protein, 122 aa, Artificial Sequence
  Synthetic Antibody
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAAGPDRWAGYDAFDVWGQGTLVTVSS

SEQ ID NO: 8 - protein, 107 aa, Artificial Sequence
  Synthetic Antibody
DIQMTQSPSSLSASVGDRVTITCRASQTINTHLNWYQQKPGKAPKLLIYGASNLQGGVPSRFSGSGSGTDFTLTISSLQPEDFATYYCQQSYSTPLTFGQGTKVEIK

SEQ ID NO: 9 - protein, 120 aa, Artificial Sequence
  Synthetic Antibody
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAAESEWDTGGFDVWGQGTLVTVSS

SEQ ID NO: 10 - protein, 106 aa, Artificial Sequence
  Synthetic Antibody
DIQMTQSPSSLSASVGDRVTITCRASQSIGSYLNWYQQKPGKAPKLLIYSSSNLQSGVPSRFSGSGSGTDFTLTISSLQPEDFATYYCQQSYTSGTFGQGTKVEIK

SEQ ID NO: 11 - protein, 117 aa, Artificial Sequence
  Synthetic Antibody
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAAEGGMGLDPWGQGTLVTVSS

SEQ ID NO: 12 - protein, 106 aa, Artificial Sequence
  Synthetic Antibody
DIQMTQSPSSLSASVGDRVTITCRASQTISSYLNWYQQKPGKAPKLLIYAASNLHSGVPSRFSGSGSGTDFTLTISSLQPEDFATYYCQQSYSSGTFGQGTKVEIK

SEQ ID NO: 13 - protein, 123 aa, Artificial Sequence
  Synthtic Antibody
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAAERGDGLDGNVGLDIWGQGTLVTVSS

SEQ ID NO: 14 - protein, 106 aa, Artificial Sequence
  Synthetic Antibody
DIQMTQSPSSLSASVGDRVTITCRASQSISKYLNWYQQKPGKAPKLLIYGASTLQSGVPSRFSGSGSGTDFTLTISSLQPEDFATYYCQQSYSSGTFGQGTKVEIK

SEQ ID NO: 15 - protein, 119 aa, Artificial Sequence
  Synthetic Antibody
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAAEASTDNGLDYWGQGTLVTVSS

SEQ ID NO: 16 - protein, 106 aa, Artificial Sequence
  Synthetic Antibody
DIQMTQSPSSLSASVGDRVTITCRASQTISRHLNWYQQKPGKAPKLLIYSASSLASGVPSRFSGSGSGTDFTLTISSLQPEDFATYYCQQSHSAGTFGQGTKVEIK

SEQ ID NO: 17 - protein, 119 aa, Artificial Sequence
  Synthetic Antibody
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAAGSGGGDGLDVWGQGTLVTVSS

SEQ ID NO: 18 - protein, 106 aa, Artificial Sequence
  Synthetic Antibody
DIQMTQSPSSLSASVGDRVTITCRASQTISSYLNWYQQKPGKAPKLLIYSSSTLQGGVPSRFSGSGSGTDFTLTISSLQPEDFATYYCQQSYSSGTFGQGTKVEIK

SEQ ID NO: 19 - protein, 118 aa, Artificial Sequence
  Synthetic Antibody
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAAETGSVGLDYWGQGTLVTVSS

SEQ ID NO: 20 - protein, 106 aa, Artificial Sequence
  Synthetic Antibody
DIQMTQSPSSLSASVGDRVTITCRASQGISTYLNWYQQKPGKAPKLLIYGATSLESGVPSRFSGSGSGTDFTLTISSLQPEDFATYYCQQGYSQGTFGQGTKVEIK

SEQ ID NO: 21 - protein, 120 aa, Artificial Sequence
  Synthetic Antibody
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAADPTEGWSYLDYWGQGTLVTVSS

SEQ ID NO: 22 - protein, 106 aa, Artificial Sequence
  Synthetic Antibody
DIQMTQSPSSLSASVGDRVTITCRASQGISSYLNWYQQKPGKAPKLLIYGASRLQSGVPSRFSGSGSGTDFTLTISSLQPEDFATYYCQQGYSSGTFGQGTKVEIK

SEQ ID NO: 23 - protein, 121 aa, Artificial Sequence
  Synthetic Antibody
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAAEANESDSGGIDVWGQGTLVTVSS

SEQ ID NO: 24 - protein, 106 aa, Artificial Sequence
  Synthetic Antibody
DIQMTQSPSSLSASVGDRVTITCRASQTIGGYLNWYQQKPGKAPKLLIYAASSLQSGVPSRFSGSGSGTDFTLTISSLQPEDFATYYCQQGYSSGAFGQGTKVEIK

SEQ ID NO: 25 - protein, 122 aa, Artificial Sequence
  Synthetic Antibody
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAAEPDGEGTNGGLDIWGQGTLVTVSS

SEQ ID NO: 26 - protein, 106 aa, Artificial Sequence
  Synthetic Antibody
DIQMTQSPSSLSASVGDRVTITCRASQNISRYLNWYQQKPGKAPKLLIYSASSLQTGVPSRFSGSGSGTDFTLTISSLQPEDFATYYCQQSYSSGAFGQGTKVEIK

SEQ ID NO: 27 - protein, 123 aa, Artificial Sequence
  Synthetic Antibody
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAAEGSGSTDGSGAIDVWGQGTLVTVSS

SEQ ID NO: 28 - protein, 106 aa, Artificial Sequence
  Synthetic Antibody
DIQMTQSPSSLSASVGDRVTITCRASQSIGSYLNWYQQKPGKAPKLLIYAASSLQSGVPSRFSGSGSGTDFTLTISSLQPEDFATYYCQQSYSSGSFGQGTKVEIK

SEQ ID NO: 29 - protein, 124 aa, Artificial Sequence
  Synthetic Antibody
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAAEGGDYPGTSDGGLDIWGQGTLVTVSS

SEQ ID NO: 30 - protein, 106 aa, Artificial Sequence
  Synthetic Antibody
DIQMTQSPSSLSASVGDRVTITCRASQSISNYLNWYQQKPGKAPKLLIYGASSLQSGVPSRFSGSGSGTDFTLTISSLQPEDFATYYCQQSYSAGTFGQGTKVEIK

SEQ ID NO: 31 - protein, 122 aa, Artificial Sequence
  Synthetic Antibody
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAAADWYSGGGDALDIWGQGTLVTVSS

SEQ ID NO: 32 - protein, 106 aa, Artificial Sequence
  Synthetic Antibody
DIQMTQSPSSLSASVGDRVTITCRASQTIYNYLNWYQQKPGKAPKLLIYGASNLQSGVPSRFSGSGSGTDFTLTISSLQPEDFATYYCQQTYSSPTFGQGTKVEIK

SEQ ID NO: 33 - protein, 126 aa, Artificial Sequence
  Synthetic Antibody
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAADQDVGSVGNYYGNGMDVWGQGTLVTVSS

SEQ ID NO: 34 - protein, 109 aa, Artificial Sequence
  Synthetic Antibody
QSVLTQPPSASGTPGQRVTISCSGSSSNIGSNYVNWYQQLPGTAPKLLIYRNNQRPSGVPDRFSGSKSGTSASLAISGLRSEDEADYYCAAWDDNPRWVFGTGTKLTVL

SEQ ID NO: 35 - protein, 119 aa, Artificial Sequence
  Synthetic Antibody
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAAEEGDGGFFDYWGQGTLVTVSS

SEQ ID NO: 36 - protein, 111 aa, Artificial Sequence
  Synthetic Antibody
QSVLTQPPSASGTPGQRVTISCSGSSSNIGSNYVNWYQQLPGTAPKLLIYRNNERPSGVPDRFSGSKSGTSASLAISGLRSEDEADYYCAAWDGSLSGRGVFGTGTKLTVL

SEQ ID NO: 37 - protein, 120 aa, Artificial Sequence
  Synthetic Antibody
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAASDYDGGGLFDLWGQGTLVTVSS

SEQ ID NO: 38 - protein, 110 aa, Artificial Sequence
  Synthetic Antibody
QSVLTQPPSASGTPGQRVTISCSGSNSNIGDNYVHWYQQLPGTAPKLLIYMNNQRPSGVPDRFSGSKSGTSASLAISGLRSEDEADYYCATWDDSLSGVVFGTGTKLTVL

SEQ ID NO: 39 - protein, 123 aa, Artificial Sequence
  Synthetic Antibody
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAADPHDGYGGGPGFDYWGQGTLVTVSS

SEQ ID NO: 40 - protein, 110 aa, Artificial Sequence
  Synthetic Antibody
QSVLTQPPSASGTPGQRVTISCSGSSSNIGSNYVYWYQQLPGTAPKLLIYRSNQRPSGVPDRFSGSKSGTSASLAISGLRSEDEADYYCAAWDDTLSAVVFGTGTKLTVL

SEQ ID NO: 41 - protein, 116 aa, Artificial Sequence
  Synthetic Antibody
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAAGLPWTDYWGQGTLVTVSS

SEQ ID NO: 42 - protein, 109 aa, Artificial Sequence
  Synthetic Antibody
QSVLTQPPSASGTPGQRVTISCSGSNSNIGSNYVSWYQQLPGTAPKLLIYSNNQRPSGVPDRFSGSKSGTSASLAISGLRSEDEADYYCAAWDDRLSVVFGTGTKLTVL

SEQ ID NO: 43 - protein, 10 aa, Artificial Sequence
  Synthetic Antibody
RRSSRSWAMH

SEQ ID NO: 44 - protein, 19 aa, Artificial Sequence
  Synthetic Antibody
VISYDGRLKYYADSVKGRF

SEQ ID NO: 45 - protein, 14 aa, Artificial Sequence
  Synthetic Antibody
ERPTSDGSTGGMDV

SEQ ID NO: 46 - protein, 11 aa, Artificial Sequence
  Synthetic Antibody
RASQSISSYLN

SEQ ID NO: 47 - protein, 7 aa, Artificial Sequence
  Synthetic Antibody
GASTLQS

SEQ ID NO: 48 - protein, 8 aa, Artificial Sequence
  Synthetic Antibody
QQSYSSGT

SEQ ID NO: 49 - protein, 15 aa, Artificial Sequence
  Synthetic Antibody
EQWGTNDGPDGGMDY

SEQ ID NO: 50 - protein, 11 aa, Artificial Sequence
  Synthetic Antibody
RASQTIGSHLN

SEQ ID NO: 51 - protein, 7 aa, Artificial Sequence
  Synthetic Antibody
GTSNLQS

SEQ ID NO: 52 - protein, 9 aa, Artificial Sequence
  Synthetic Antibody
QQSYSSPLT

SEQ ID NO: 53 - protein, 13 aa, Artificial Sequence
  Synthetic Antibody
GPDRWAGYDAFDV

SEQ ID NO: 54 - protein, 11 aa, Artificial Sequence
  Synthetic Antibody
RASQTINTHLN

SEQ ID NO: 55 - protein, 7 aa, Artificial Sequence
  Synthetic Antibody
GASNLQG

SEQ ID NO: 56 - protein, 9 aa, Artificial Sequence
  Synthetic Antibody
QQSYSTPLT

SEQ ID NO: 57 - protein, 11 aa, Artificial Sequence
  Synthetic Antibody
ESEWDTGGFDV

SEQ ID NO: 58 - protein, 11 aa, Artificial Sequence
  Synthetic Antibody
RASQSIGSYLN

SEQ ID NO: 59 - protein, 7 aa, Artificial Sequence
  Synthetic Antibody
SSSNLQS

SEQ ID NO: 60 - protein, 8 aa, Artificial Sequence
  Synthetic Antibody
QQSYTSGT

SEQ ID NO: 61 - protein, 8 aa, Artificial Sequence
  Synthetic Antibody
EGGMGLDP

SEQ ID NO: 62 - protein, 11 aa, Artificial Sequence
  Synthetic Antibody
RASQTISSYLN

SEQ ID NO: 63 - protein, 7 aa, Artificial Sequence
  Synthetic Antibody
AASNLHS

SEQ ID NO: 64 - protein, 14 aa, Artificial Sequence
  Synthetic Antibody
ERGDGLDGNVGLDI

SEQ ID NO: 65 - protein, 11 aa, Artificial Sequence
  Synthetic Antibody
RASQSISKYLN

SEQ ID NO: 66 - protein, 10 aa, Artificial Sequence
  Synthetic Antibody
EASTDNGLDY

SEQ ID NO: 67 - protein, 11 aa, Artificial Sequence
  Synthetic Antibody
RASQTISRHLN

SEQ ID NO: 68 - protein, 7 aa, Artificial Sequence
  Synthetic Antibody
SASSLAS

SEQ ID NO: 69 - protein, 8 aa, Artificial Sequence
  Synthetic Antibody
QQSHSAGT

SEQ ID NO: 70 - protein, 10 aa, Artificial Sequence
  Synthetic Antibody
GSGGGDGLDV

SEQ ID NO: 71 - protein, 7 aa, Artificial Sequence
  Synthetic Antibody
SSSTLQG

SEQ ID NO: 72 - protein, 9 aa, Artificial Sequence
  Synthetic Antibody
ETGSVGLDY

SEQ ID NO: 73 - protein, 11 aa, Artificial Sequence
  Synthetic Antibody
RASQGISTYLN

SEQ ID NO: 74 - protein, 7 aa, Artificial Sequence
  Synthetic Antibody
GATSLES

SEQ ID NO: 75 - protein, 8 aa, Artificial Sequence
  Synthetic Antibody
QQGYSQGT

SEQ ID NO: 76 - protein, 11 aa, Artificial Sequence
  Synthetic Antibody
DPTEGWSYLDY

SEQ ID NO: 77 - protein, 11 aa, Artificial Sequence
  Synthetic Antibody
RASQGISSYLN

SEQ ID NO: 78 - protein, 7 aa, Artificial Sequence
  Synthetic Antibody
GASRLQS

SEQ ID NO: 79 - protein, 8 aa, Artificial Sequence
  Synthetic Antibody
QQGYSSGT

SEQ ID NO: 80 - protein, 12 aa, Artificial Sequence
  Synthetic Antibody
EANESDSGGIDV

SEQ ID NO: 81 - protein, 11 aa, Artificial Sequence
  Synthetic Antibody
RASQTIGGYLN

SEQ ID NO: 82 - protein, 7 aa, Artificial Sequence
  Synthetic Antibody
AASSLQS

SEQ ID NO: 83 - protein, 8 aa, Artificial Sequence
  Synthetic Antibody
QQGYSSGA

SEQ ID NO: 84 - protein, 13 aa, Artificial Sequence
  Synthetic Antibody
EPDGEGTNGGLDI

SEQ ID NO: 85 - protein, 11 aa, Artificial Sequence
  Synthetic Antibody
RASQNISRYLN

SEQ ID NO: 86 - protein, 7 aa, Artificial Sequence
  Synthetic Antibody
SASSLQT

SEQ ID NO: 87 - protein, 8 aa, Artificial Sequence
  Synthetic Antibody
QQSYSSGA

SEQ ID NO: 88 - protein, 14 aa, Artificial Sequence
  Synthetic Antibody
EGSGSTDGSGAIDV

SEQ ID NO: 89 - protein, 8 aa, Artificial Sequence
  Synthetic Antibody
QQSYSSGS

SEQ ID NO: 90 - protein, 15 aa, Artificial Sequence
  Synthetic Antibody
EGGDYPGTSDGGLDI

SEQ ID NO: 91 - protein, 11 aa, Artificial Sequence
  Synthetic Antibody
RASQSISNYLN

SEQ ID NO: 92 - protein, 7 aa, Artificial Sequence
  Synthetic Antibody
GASSLQS

SEQ ID NO: 93 - protein, 8 aa, Artificial Sequence
  Synthetic Antibody
QQSYSAGT

SEQ ID NO: 94 - protein, 13 aa, Artificial Sequence
  Synthetic Antibody
ADWYSGGGDALDI

SEQ ID NO: 95 - protein, 11 aa, Artificial Sequence
  Synthetic Antibody
RASQTIYNYLN

SEQ ID NO: 96 - protein, 7 aa, Artificial Sequence
  Synthetic Antibody
RNNQRPS

SEQ ID NO: 97 - protein, 8 aa, Artificial Sequence
  Synthetic Antibody
QQTYSSPT

SEQ ID NO: 98 - protein, 17 aa, Artificial Sequence
  Synthetic Antibody
DQDVGSVGNYYGNGMDV

SEQ ID NO: 99 - protein, 13 aa, Artificial Sequence
  Synthetic Antibody
SGSSSNIGSNYVN

SEQ ID NO: 100 - protein, 10 aa, Artificial Sequence
  Synthetic Antibody
AAWDDNPRWV

SEQ ID NO: 101 - protein, 10 aa, Artificial Sequence
  Synthetic Antibody
EEGDGGFFDY

SEQ ID NO: 102 - protein, 7 aa, Artificial Sequence
  Synthetic Antibody
RNNERPS

SEQ ID NO: 103 - protein, 12 aa, Artificial Sequence
  Synthetic Antibody
AAWDGSLSGRGV

SEQ ID NO: 104 - protein, 11 aa, Artificial Sequence
  Synthetic Antibody
SDYDGGGLFDL

SEQ ID NO: 105 - protein, 13 aa, Artificial Sequence
  Synthetic Antibody
SGSNSNIGDNYVH

SEQ ID NO: 106 - protein, 7 aa, Artificial Sequence
  Synthetic Antibody
MNNQRPS

SEQ ID NO: 107 - protein, 11 aa, Artificial Sequence
  Synthetic Antibody
ATWDDSLSGVV

SEQ ID NO: 108 - protein, 14 aa, Artificial Sequence
  Synthetic Antibody
DPHDGYGGGPGFDY

SEQ ID NO: 109 - protein, 13 aa, Artificial Sequence
  Synthetic Antibody
SGSSSNIGSNYVY

SEQ ID NO: 110 - protein, 7 aa, Artificial Sequence
  Synthetic Antibody
RSNQRPS

SEQ ID NO: 111 - protein, 11 aa, Artificial Sequence
  Synthetic Antibody
AAWDDTLSAVV

SEQ ID NO: 112 - protein, 7 aa, Artificial Sequence
  Synthetic Antibody
GLPWTDY

SEQ ID NO: 113 - protein, 13 aa, Artificial Sequence
  Synthetic Antibody
SGSNSNIGSNYVS

SEQ ID NO: 114 - protein, 7 aa, Artificial Sequence
  Synthetic Antibody
SNNQRPS

SEQ ID NO: 115 - protein, 10 aa, Artificial Sequence
  Synthetic Antibody
AAWDDRLSVV

SEQ ID NO: 116 - protein, 7 aa, Artificial Sequence
  Synthetic Antibody
RRSSRSW

SEQ ID NO: 117 - protein, 5 aa, Artificial Sequence
  Synthetic Antibody
SWAMH

SEQ ID NO: 118 - protein, 6 aa, Artificial Sequence
  Synthetic Antibody
SYDGRL

SEQ ID NO: 119 - protein, 126 aa, Artificial Sequence
  Synthetic Antibody
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAADVESALAYGEPYHHMDVWGQGTLVTVSS

SEQ ID NO: 120 - protein, 110 aa, Artificial Sequence
  Synthetic Antibody
QSVLTQPPSASGTPGQRVTISCSGSSSNIGRNYVYWYQQLPGTAPKLLIYRTSQRPSGVPDRFSGSKSGTSASLAISGLRSEDEADYYCATWDDSLSGWVFGTGTKLTVL

SEQ ID NO: 121 - protein, 126 aa, Artificial Sequence
  Synthetic Antibody
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAAGFDWGWDSSDIYSAMDYWGQGTLVTVSS

SEQ ID NO: 122 - protein, 107 aa, Artificial Sequence
  Synthetic Antibody
DIQMTQSPSSLSASVGDRVTITCRASQNIGRYLHWYQQKPGKAPKLLIYSASNLQSGVPSRFSGSGSGTDFTLTISSLQPEDFATYYCQQSYSSPFTFGQGTKVEIK

SEQ ID NO: 123 - protein, 126 aa, Artificial Sequence
  Synthetic Antibody
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAADDDGDVYAGYSYGGFDYWGQGTLVTVSS

SEQ ID NO: 124 - protein, 110 aa, Artificial Sequence
  Synthetic Antibody
QSVLTQPPSASGTPGQRVTISCSGSSSNIGRNYVYWYQQLPGTAPKLLIYRNDQRPSGVPDRFSGSKSGTSASLAISGLRSEDEADYYCAAWDDSLSGWVFGTGTKLTVL

SEQ ID NO: 125 - protein, 124 aa, Artificial Sequence
  Synthetic Antibody
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAAAPRVDWSDVSSGIDYWGQGTLVTVSS

SEQ ID NO: 126 - protein, 110 aa, Artificial Sequence
  Synthetic Antibody
QSVLTQPPSASGTPGQRVTISCSGSISNIGTNNVYWYQQLPGTAPKLLIYRNNQRPSGVPDRFSGSKSGTSASLAISGLRSEDEADYYCSAWDDSLSGVVFGTGTKLTVL

SEQ ID NO: 127 - protein, 123 aa, Artificial Sequence
  Synthetic Antibody
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAADMDDMLSYYNSFDIWGQGTLVTVSS

SEQ ID NO: 128 - protein, 110 aa, Artificial Sequence
  Synthetic Antibody
QSVLTQPPSASGTPGQRVTISCSGSSSNIGRNYVYWYQQLPGTAPKLLIYRNNQRPSGVPDRFSGSKSGTSASLAISGLRSEDEADYYCAAWDDSLSGWVFGTGTKLTVL

SEQ ID NO: 129 - protein, 122 aa, Artificial Sequence
  Synthetic Antibody
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAADTSMFASYDPMDVWGQGTLVTVSS

SEQ ID NO: 130 - protein, 110 aa, Artificial Sequence
  Synthetic Antibody
QSVLTQPPSASGTPGQRVTISCSGSSSNIGRNYVNWYQQLPGTAPKLLIYRDDHRPSGVPDRFSGSKSGTSASLAISGLRSEDEADYYCASWDDTLSGWVFGTGTKLTVL

SEQ ID NO: 131 - protein, 124 aa, Artificial Sequence
  Synthetic Antibody
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAADGKGAAWSEVDEPDVWGQGTLVTVSS

SEQ ID NO: 132 - protein, 110 aa, Artificial Sequence
  Synthetic Antibody
QSVLTQPPSASGTPGQRVTISCSGSSSNIGSNYVYWYQQLPGTAPKLLIYQNNQRPSGVPDRFSGSKSGTSASLAISGLRSEDEADYYCAAWDDSLSGLVFGTGTKLTVL

SEQ ID NO: 133 - protein, 126 aa, Artificial Sequence
  Synthetic Antibody
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAASDDADIFDDYSYGAMDYWGQGTLVTVSS

SEQ ID NO: 134 - protein, 110 aa, Artificial Sequence
  Synthetic Antibody
QSVLTQPPSASGTPGQRVTISCSGSSSNIGRNYVYWYQQLPGTAPKLLIYMTSQRPSGVPDRFSGSKSGTSASLAISGLRSEDEADYYCASWDDNLRAWVFGTGTKLTVL

SEQ ID NO: 135 - protein, 123 aa, Artificial Sequence
  Synthetic Antibody
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAAARDVDGSGYDTIDVWGQGTLVTVSS

SEQ ID NO: 136 - protein, 111 aa, Artificial Sequence
  Synthetic Antibody
QSVLTQPPSASGTPGQRVTISCSGSSSNIGRNYVYWYQQLPGTAPKLLIYSNNQRPSGVPDRFSGSKSGTSASLAISGLRSEDEADYYCAAWDDSLSGVWVFGTGTKLTVL

SEQ ID NO: 137 - protein, 17 aa, Artificial Sequence
  Synthetic Antibody
DVESALAYGEPYHHMDV

SEQ ID NO: 138 - protein, 13 aa, Artificial Sequence
  Synthetic Antibody
SGSSSNIGRNYVY

SEQ ID NO: 139 - protein, 7 aa, Artificial Sequence
  Synthetic Antibody
RTSQRPS

SEQ ID NO: 140 - protein, 11 aa, Artificial Sequence
  Synthetic Antibody
ATWDDSLSGWV

SEQ ID NO: 141 - protein, 17 aa, Artificial Sequence
  Synthetic Antibody
GFDWGWDSSDIYSAMDY

SEQ ID NO: 142 - protein, 11 aa, Artificial Sequence
  Synthetic Antibody
RASQNIGRYLH

SEQ ID NO: 143 - protein, 7 aa, Artificial Sequence
  Synthetic Antibody
SASNLQS

SEQ ID NO: 144 - protein, 9 aa, Artificial Sequence
  Synthetic Antibody
QQSYSSPFT

SEQ ID NO: 145 - protein, 17 aa, Artificial Sequence
  Synthetic Antibody
DDDGDVYAGYSYGGFDY

SEQ ID NO: 146 - protein, 7 aa, Artificial Sequence
  Synthetic Antibody
RNDQRPS

SEQ ID NO: 147 - protein, 11 aa, Artificial Sequence
  Synthetic Antibody
AAWDDSLSGWV

SEQ ID NO: 148 - protein, 15 aa, Artificial Sequence
  Synthetic Antibody
APRVDWSDVSSGIDY

SEQ ID NO: 149 - protein, 13 aa, Artificial Sequence
  Synthetic Antibody
SGSISNIGTNNVY

SEQ ID NO: 150 - protein, 11 aa, Artificial Sequence
  Synthetic Antibody
SAWDDSLSGVV

SEQ ID NO: 151 - protein, 14 aa, Artificial Sequence
  Synthetic Antibody
DMDDMLSYYNSFDI

SEQ ID NO: 152 - protein, 13 aa, Artificial Sequence
  Synthetic Antibody
DTSMFASYDPMDV

SEQ ID NO: 153 - protein, 13 aa, Artificial Sequence
  Synthetic Antibody
SGSSSNIGRNYVN

SEQ ID NO: 154 - protein, 7 aa, Artificial Sequence
  Synthetic Antibody
RDDHRPS

SEQ ID NO: 155 - protein, 11 aa, Artificial Sequence
  Synthetic Antibody
ASWDDTLSGWV

SEQ ID NO: 156 - protein, 15 aa, Artificial Sequence
  Synthetic Antibody
DGKGAAWSEVDEPDV

SEQ ID NO: 157 - protein, 7 aa, Artificial Sequence
  Synthetic Antibody
QNNQRPS

SEQ ID NO: 158 - protein, 11 aa, Artificial Sequence
  Synthetic Antibody
AAWDDSLSGLV

SEQ ID NO: 159 - protein, 17 aa, Artificial Sequence
  Synthetic Antibody
SDDADIFDDYSYGAMDY

SEQ ID NO: 160 - protein, 7 aa, Artificial Sequence
  Synthetic Antibody
MTSQRPS

SEQ ID NO: 161 - protein, 11 aa, Artificial Sequence
  Synthetic Antibody
ASWDDNLRAWV

SEQ ID NO: 162 - protein, 14 aa, Artificial Sequence
  Synthetic Antibody
ARDVDGSGYDTIDV

SEQ ID NO: 163 - protein, 12 aa, Artificial Sequence
  Synthetic Antibody
AAWDDSLSGVWV

SEQ ID NO: 164 - protein, 7 aa, Artificial Sequence
  Synthetic Antibody
GASNLQS

SEQ ID NO: 165 - protein, 4 aa, Artificial Sequence
  Synthetic Antibody
YLVY

SEQ ID NO: 166 - protein, 126 aa, Artificial Sequence
  Synthetic Antibody
  MISC_FEATURE (99)..(115): Xaa at positions 99-115 may be any naturally-occurring amino acid and up to eleven of them may be absent
QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAAXXXXXXXXXXXXXXXXXWGQGTLVTVSS

(19) United States
(12) Patent Application Publication
(10) Pub. No.: US 2023/0192839 A1
(43) Pub. Date: Jun. 22, 2023

(54) ANTIBODIES HAVING CONDITIONAL AFFINITY AND METHODS OF USE THEREOF

(71) Applicant: PFIZER INC., NEW YORK, NY (US)

(72) Inventors: Bijan Andre BOLDAJIPOUR, San Francisco, CA (US); Javier Fernando CHAPARRO RIGGERS, San Mateo, CA (US); Xiaodi DENG, San Mateo, CA (US); Kevin Charles LINDQUIST, San Diego, CA (US); Spencer PARK, Seattle, WA (US); Edward Derrick PASCUA, San Diego, CA (US); Thomas John VAN BLARCOM, Oakland, CA (US)

(73) Assignee: PFIZER INC., NEW YORK, NY (US)

(21) Appl. No.: 16/500,970

(22) PCT Filed: Mar. 29, 2018
(86) PCT No.: PCT/IB2018/052199

§ 371 Date: Oct. 4, 2019

Related U.S. Application Data
(60) Provisional application No. 62/484,776, filed on Apr. 12, 2017.
(60) Provisional application No. 62/637,077, filed on Mar. 1, 2018.

(51) Int. Cl. C07K 16/28; C07K 16/00; C12N 15/63

(52) U.S. Cl. CPC C07K 16/28; C07K 16/005; C12N 15/63; C07K 2317/92; C07K 2317/565; C07K 2317/622; C07K 2319/03

(57) ABSTRACT

Provided herein are antibodies with conditional affinity for an antigen. The affinity of an antibody with conditional affinity for an antigen may be altered by a small molecule agent. Antibodies with conditional affinity may be used therapeutically alone or in combination with other therapeutics to treat cancer and other diseases.

This application is a §371 filing of PCT/IB2018/052199 filed Mar. 29, 2018, which claims the benefit of priority to U.S. Provisional Application Nos. 62/484,776 filed Apr. 12, 2017 and 62/637,077 filed Mar. 1, 2018; the entire contents of which are incorporated herein by reference.

### REFERENCE TO SEQUENCE LISTING

This application is being filed electronically via EFS-Web and includes an electronically submitted sequence listing in .txt format. The .txt file contains a sequence listing entitled “PC72330A_SEQ_LISTING_Corrected_ST25.txt” created on Mar. 23, 2020 and having a size of 87 KB. The sequence listing contained in this .txt file is part of the specification and is herein incorporated by reference in its entirety.

### FIELD

Embodiments provided herein relate to antibodies that have conditional antigen affinity. The affinity of these antibodies for an antigen may be altered, for example, by the concentration of a particular small molecule in the antibody environment. Also provided are related compositions and methods.

### BACKGROUND

Antibodies are valuable therapeutic, diagnostic, and research tools. Frequently, when selecting an antibody against an antigen of interest, it is desirable to obtain or use an antibody that has a high affinity for the antigen under most or all circumstances, so that the antibody will stably bind to the antigen. Stable binding of the antibody to the antigen typically promotes a desired downstream event from the antibody-antigen binding (e.g. generation of a therapeutic effect or a detectable signal, etc.).

In certain circumstances, however, it may be desirable to use an antibody that can have a high affinity for an antigen, but that does not always have high affinity for the antigen – i.e. to have an antibody that could be “turned on” or “turned off” for antigen binding, depending on one or more conditions in the antibody-antigen environment. Such antibodies could be used, for example, to bind and then release an antigen of interest for purposes of purifying the antigen, or to bind an antigen in a subject only under certain circumstances.

It is known that many antibodies have reduced affinity for antigens at strongly acidic conditions (e.g. antibodies can frequently be dissociated from antigens by exposure of an antibody-antigen complex to pH 2.5 conditions). However low pH conditions are non-selective, are generally detrimental to the antibody, antigen, and other molecules, and cannot be used in living organisms.

Accordingly, there is a need for improved antibodies that have conditional affinity for an antigen.

### SUMMARY

Provided herein are antibodies which have conditional affinity for binding to an antigen, such that the affinity of the antibody for an antigen is affected by an environmental condition. The environmental condition may be, for example, the concentration of a small molecule agent. An antibody as described herein which has different affinities for an antigen, depending on an environmental condition, may be referred to herein as an antibody “with conditional affinity”, “having conditional affinity”, or the like.

In some embodiments, an antibody with conditional affinity provided herein may also specifically bind a small molecule agent. Thus, in some embodiments, an antibody provided herein may be capable of binding both i) an antigen, and ii) a small molecule agent, and the binding of the small molecule agent to the antibody may affect the affinity of the antibody for the antigen.

Antibodies with conditional affinity for an antigen as provided herein may be useful for various purposes.

In an example, an antibody which has a greater affinity for an antigen in the presence of a high concentration condition of a small molecule agent may be “activated” to bind to the antigen by exposing the antibody to a high concentration condition of the small molecule agent (i.e. the antibody effectively has an “on switch” which can be turned on by the small molecule agent). In some embodiments, the small molecule agent may be a drug which can be administered to a subject, and administration of the drug to the subject may yield a high concentration condition of the small molecule agent in the subject. If the subject is also administered an antibody with conditional affinity as provided herein which has a greater affinity for an antigen in the presence of a high concentration condition of the small molecule agent as compared to a low concentration condition of the small molecule agent, then the presence of a high concentration condition of the small molecule agent in the subject (caused, for example, by the administration of the small molecule agent to the subject) will cause the antibody with conditional affinity to have a greater affinity for its antigen than when the antibody is in a low concentration condition of the small molecule agent. Thus, in the situation described immediately above, administration of the small molecule agent to the subject may cause an increase in the binding of the antibody to its antigen in the subject. In this way, the antibody may be effectively “activated” or “turned on” to bind to an antigen by the administration of the small molecule agent to the subject. This may be useful, for example, when it is desirable to only have the antibody bind to the antigen in the subject at certain selected times / under certain selected conditions, and when it is desirable to start or increase an antibody binding to its antigen.

In another example, an antibody which has lower affinity for an antigen in the presence a high concentration condition of a small molecule agent maybe “deactivated” / “inhibited” from binding to the antigen by exposing the antibody to a high concentration condition of the small molecule agent (i.e. the antibody effectively has an “off switch” which can be turned off by the small molecule agent). In some embodiments, the small molecule agent may be a drug which can be administered to a subject, and administration of the drug to the subject may yield a high concentration condition of the small molecule agent in the subject. If the subject is also administered an antibody with conditional affinity as provided herein which has a lower affinity for an antigen in the presence of a high concentration condition of the small molecule agent as compared to a high concentration condition of the small molecule agent, then the presence of a high concentration condition of the small molecule agent in the subject (caused, for example, by the administration of the small molecule agent to the subject) will cause the antibody with conditional affinity to have a lower affinity for its antigen than when the antibody is in a low concentration condition of the small molecule agent. Thus, in the situation described immediately above, administration of the small molecule agent to the subject may cause a decrease in the binding of the antibody to the antigen in the subject. In this way, the antibody may be effectively “deactivated” / “turned off” from binding to the antigen by the administration of the small molecule agent to the subject. This may be useful, for example, when it is desirable to only have the antibody bind to its antigen in the subject at certain selected times / under certain selected conditions, and when it is desirable to stop or decrease an antibody from binding to an antigen.

In some embodiments, provided herein is an antibody with conditional affinity which specifically binds an antigen with higher affinity under a low agent concentration condition than under a high agent concentration condition, wherein the antibody comprises a heavy chain variable region (VH) and a light chain variable region (VL); the antibody comprises an agent-binding motif within the VH or VL; and the agent is a small molecule.

In some embodiments, provided herein is an antibody with conditional affinity which specifically binds an antigen with higher affinity under a high agent concentration condition than under a low agent concentration condition, wherein the antibody comprises a heavy chain variable region (VH) and a light chain variable region (VL); the antibody comprises an agent-binding motif within the VH or VL; and the agent is a small molecule.

In some embodiments, provided herein is an antibody with conditional affinity which specifically binds an antigen with higher affinity under a low agent concentration condition than under a high agent concentration condition, wherein the antibody comprises a heavy chain variable region (VH) and a light chain variable region (VL); and the agent is a small molecule.

In some embodiments, provided herein is an antibody with conditional affinity which specifically binds an antigen with higher affinity under a high agent concentration condition than under a low agent concentration condition, wherein the antibody comprises a heavy chain variable region (VH) and a light chain variable region (VL); and the agent is a small molecule.

In some embodiments, provided herein is an antibody with conditional affinity which specifically binds an antigen with higher affinity under a low small molecule agent concentration than under a high small molecule agent concentration, wherein: a) the antibody comprises a heavy chain variable region (VH) and a light chain variable region (VL), wherein the VH comprises a CDR1 and a CDR2 of the partial VH sequence shown in SEQ ID NO: 1; and b) the small molecule agent is methotrexate. Optionally, the VH comprises an amino acid sequence as shown in SEQ ID NO: 1. Optionally, the VH comprises an amino acid sequence as shown in SEQ ID NO: 166.

In some embodiments, provided herein is an antibody with conditional affinity which specifically binds an antigen with higher affinity under a high agent concentration than under a low agent concentration, wherein: a) the antibody comprises a heavy chain variable region (VH) and a light chain variable region (VL), wherein the VH comprises a CDR1 and a CDR2 of the partial VH sequence shown in SEQ ID NO: 1; and b) the small molecule agent is methotrexate. Optionally, the VH comprises an amino acid sequence as shown in SEQ ID NO: 1. Optionally, the VH comprises an amino acid sequence as shown in SEQ ID NO: 166.

In some embodiments, provided herein is an antibody with conditional affinity which specifically binds an antigen with higher affinity under a low agent concentration than under a high agent concentration, wherein: a) the antibody comprises a heavy chain variable region (VH) and a light chain variable region (VL), wherein the VH comprises a CDR1, CDR2, and CDR3 of a VH sequence as shown in any one of SEQ ID NOs: 3, 7, 9, 13, 15, 17, 19, 21, 23, 25, 27, 29, 33, 35, 37, 39, 41, 119, 121, 123, 125, 127, 133 or 135; and b) the small molecule agent is methotrexate. In some embodiments, provided herein is an antibody with conditional affinity which specifically binds an antigen with higher affinity under a low agent concentration than under a high agent concentration, wherein: a) the antibody comprises a heavy chain variable region (VH) and a light chain variable region (VL), wherein the VH comprises a VH sequence as shown in any one of SEQ ID NOs: 3, 7, 9, 13, 15, 17, 19, 21, 23, 25, 27, 29, 33, 35, 37, 39, 41, 119, 121, 123, 125, 127, 133 or 135; and b) the small molecule agent is methotrexate.

In some embodiments, provided herein is an antibody with conditional affinity which specifically binds an antigen with higher affinity under a high agent concentration than under a low agent concentration, wherein: a) the antibody comprises a heavy chain variable region (VH) and a light chain variable region (VL), wherein the VH comprises a CDR1, CDR2, and CDR3 of a VH sequence as shown in SEQ ID NO: 31; and b) the small molecule agent is methotrexate. In some embodiments, provided herein is an antibody with conditional affinity which specifically binds an antigen with higher affinity under a high agent concentration than under a low agent concentration, wherein: a) the antibody comprises a heavy chain variable region (VH) and a light chain variable region (VL), wherein the VH comprises a VH sequence as shown in SEQ ID NO: 31; and b) the small molecule agent is methotrexate.

In some embodiments, provided herein is an antibody with conditional affinity comprising a heavy chain variable region (VH) and a light chain variable region (VL), wherein the antibody comprises a VH CDR1, VH CDR2, and VH CDR3 of a VH sequence of a clone provided in Table 3 or Table 6 herein, and a VL CDR1, VL CDR2, and VL CDR3 of a VL sequence of the same respective clone provided in Table 3 or Table 6. In some embodiments, provided herein is an antibody with conditional affinity comprising a VH and a VL, wherein the antibody comprises a VH sequence of a clone provided in Table 3 or Table 6 herein, and a VL sequence of the same respective clone provided in Table 3 or Table 6.

In some embodiments, provided herein is an antibody with conditional affinity comprising a VH and a VL, wherein any one of: the VH comprises a VH CDR1, VH CDR2, and VH CDR3 of a VH sequence as shown in SEQ ID NO: 3 and the VL comprises a VL CDR1, VL CDR2, and VL CDR3 of a VL sequence as shown in SEQ ID NO: 4; the VH comprises a VH CDR1, VH CDR2, and VH CDR3 of a VH sequence as shown in SEQ ID NO: 7 and the VL comprises a VL CDR1, VL CDR2, and VL CDR3 of a VL sequence as shown in SEQ ID NO: 8; the VH comprises a VH CDR1, VH CDR2, and VH CDR3 of a VH sequence as shown in SEQ ID NO: 9 and the VL comprises a VL CDR1, VL CDR2, and VL CDR3 of a VL sequence as shown in SEQ ID NO: 10; the VH comprises a VH CDR1, VH CDR2, and VH CDR3 of a VH sequence as shown in SEQ ID NO: 13 and the VL comprises a VL CDR1, VL CDR2, and VL CDR3 of a VL sequence as shown in SEQ ID NO: 14; the VH comprises a VH CDR1, VH CDR2, and VH CDR3 of a VH sequence as shown in SEQ ID NO: 15 and the VL comprises a VL CDR1, VL CDR2, and VL CDR3 of a VL sequence as shown in SEQ ID NO: 16; the VH comprises a VH CDR1, VH CDR2, and VH CDR3 of a VH sequence as shown in SEQ ID NO: 17 and the VL comprises a VL CDR1, VL CDR2, and VL CDR3 of a VL sequence as shown in SEQ ID NO: 18; the VH comprises a VH CDR1, VH CDR2, and VH CDR3 of a VH sequence as shown in SEQ ID NO: 19 and the VL comprises a VL CDR1, VL CDR2, and VL CDR3 of a VL sequence as shown in SEQ ID NO: 20; the VH comprises a VH CDR1, VH CDR2, and VH CDR3 of a VH sequence as shown in SEQ ID NO: 21 and the VL comprises a VL CDR1, VL CDR2, and VL CDR3 of a VL sequence as shown in SEQ ID NO: 22; the VH comprises a VH CDR1, VH CDR2, and VH CDR3 of a VH sequence as shown in SEQ ID NO: 23 and the VL comprises a VL CDR1, VL CDR2, and VL CDR3 of a VL sequence as shown in SEQ ID NO: 24; the VH comprises a VH CDR1, VH CDR2, and VH CDR3 of a VH sequence as shown in SEQ ID NO: 25 and the VL comprises a VL CDR1, VL CDR2, and VL CDR3 of a VL sequence as shown in SEQ ID NO: 26; the VH comprises a VH CDR1, VH CDR2, and VH CDR3 of a VH sequence as shown in SEQ ID NO: 27 and the VL comprises a VL CDR1, VL CDR2, and VL CDR3 of a VL sequence as shown in SEQ ID NO: 28; the VH comprises a VH CDR1, VH CDR2, and VH CDR3 of a VH sequence as shown in SEQ ID NO: 29 and the VL comprises a VL CDR1, VL CDR2, and VL CDR3 of a VL sequence as shown in SEQ ID NO: 30; the VH comprises a VH CDR1, VH CDR2, and VH CDR3 of a VH sequence as shown in SEQ ID NO: 31 and the VL comprises a VL CDR1, VL CDR2, and VL CDR3 of a VL sequence as shown in SEQ ID NO: 32; the VH comprises a VH CDR1, VH CDR2, and VH CDR3 of a VH sequence as shown in SEQ ID NO: 33 and the VL comprises a VL CDR1, VL CDR2, and VL CDR3 of a VL sequence as shown in SEQ ID NO: 34; the VH comprises a VH CDR1, VH CDR2, and VH CDR3 of a VH sequence as shown in SEQ ID NO: 35 and the VL comprises a VL CDR1, VL CDR2, and VL CDR3 of a VL sequence as shown in SEQ ID NO: 36; the VH comprises a VH CDR1, VH CDR2, and VH CDR3 of a VH sequence as shown in SEQ ID NO: 37 and the VL comprises a VL CDR1, VL CDR2, and VL CDR3 of a VL sequence as shown in SEQ ID NO: 38; the VH comprises a VH CDR1, VH CDR2, and VH CDR3 of a VH sequence as shown in SEQ ID NO: 39 and the VL comprises a VL CDR1, VL CDR2, and VL CDR3 of a VL sequence as shown in SEQ ID NO: 40; the VH comprises a VH CDR1, VH CDR2, and VH CDR3 of a VH sequence as shown in SEQ ID NO: 41 and the VL comprises a VL CDR1, VL CDR2, and VL CDR3 of a VL sequence as shown in SEQ ID NO: 42; the VH comprises a VH CDR1, VH CDR2, and VH CDR3 of a VH sequence as shown in SEQ ID NO: 119 and the VL comprises a VL CDR1, VL CDR2, and VL CDR3 of a VL sequence as shown in SEQ ID NO: 120; the VH comprises a VH CDR1, VH CDR2, and VH CDR3 of a VH sequence as shown in SEQ ID NO: 121 and the VL comprises a VL CDR1, VL CDR2, and VL CDR3 of a VL sequence as shown in SEQ ID NO: 122; the VH comprises a VH CDR1, VH CDR2, and VH CDR3 of a VH sequence as shown in SEQ ID NO: 123 and the VL comprises a VL CDR1, VL CDR2, and VL CDR3 of a VL sequence as shown in SEQ ID NO: 124; the VH comprises a VH CDR1, VH CDR2, and VH CDR3 of a VH sequence as shown in SEQ ID NO: 125 and the VL comprises a VL CDR1, VL CDR2, and VL CDR3 of a VL sequence as shown in SEQ ID NO: 126; the VH comprises a VH CDR1, VH CDR2, and VH CDR3 of a VH sequence as shown in SEQ ID NO: 127 and the VL comprises a VL CDR1, VL CDR2, and VL CDR3 of a VL sequence as shown in SEQ ID NO: 128; the VH comprises a VH CDR1, VH CDR2, and VH CDR3 of a VH sequence as shown in SEQ ID NO: 133 and the VL comprises a VL CDR1, VL CDR2, and VL CDR3 of a VL sequence as shown in SEQ ID NO: 134; or the VH comprises a VH CDR1, VH CDR2, and VH CDR3 of a VH sequence as shown in SEQ ID NO: 135 and the VL comprises a VL CDR1, VL CDR2, and VL CDR3 of a VL sequence as shown in SEQ ID NO: 136.

In some embodiments, provided herein is an antibody with conditional affinity comprising a VH and a VL, wherein any one of: the VH comprises a VH sequence as shown in SEQ ID NO: 3 and the VL comprises a VL sequence as shown in SEQ ID NO: 4; the VH comprises a VH sequence as shown in SEQ ID NO: 7 and the VL comprises a VL sequence as shown in SEQ ID NO: 8; the VH comprises a VH sequence as shown in SEQ ID NO: 9 and the VL comprises a VL sequence as shown in SEQ ID NO: 10; the VH comprises a VH sequence as shown in SEQ ID NO: 13 and the VL comprises a VL sequence as shown in SEQ ID NO: 14; the VH comprises a VH sequence as shown in SEQ ID NO: 15 and the VL comprises a VL sequence as shown in SEQ ID NO: 16; the VH comprises a VH sequence as shown in SEQ ID NO: 17 and the VL comprises a VL sequence as shown in SEQ ID NO: 18; the VH comprises a VH sequence as shown in SEQ ID NO: 19 and the VL comprises a VL sequence as shown in SEQ ID NO: 20; the VH comprises a VH sequence as shown in SEQ ID NO: 21 and the VL comprises a VL sequence as shown in SEQ ID NO: 22; the VH comprises a VH sequence as shown in SEQ ID NO: 23 and the VL comprises a VL sequence as shown in SEQ ID NO: 24; the VH comprises a VH sequence as shown in SEQ ID NO: 25 and the VL comprises a VL sequence as shown in SEQ ID NO: 26; the VH comprises a VH sequence as shown in SEQ ID NO: 27 and the VL comprises a VL sequence as shown in SEQ ID NO: 28; the VH comprises a VH sequence as shown in SEQ ID NO: 29 and the VL comprises a VL sequence as shown in SEQ ID NO: 30; the VH comprises a VH sequence as shown in SEQ ID NO: 31 and the VL comprises a VL sequence as shown in SEQ ID NO: 32; the VH comprises a VH sequence as shown in SEQ ID NO: 33 and the VL comprises a VL sequence as shown in SEQ ID NO: 34; the VH comprises a VH sequence as shown in SEQ ID NO: 35 and the VL comprises a VL sequence as shown in SEQ ID NO: 36; the VH comprises a VH sequence as shown in SEQ ID NO: 37 and the VL comprises a VL sequence as shown in SEQ ID NO: 38; the VH comprises a VH sequence as shown in SEQ ID NO: 39 and the VL comprises a VL sequence as shown in SEQ ID NO: 40; the VH comprises a VH sequence as shown in SEQ ID NO: 41 and the VL comprises a VL sequence as shown in SEQ ID NO: 42; the VH comprises a VH sequence as shown in SEQ ID NO: 119 and the VL comprises a VL sequence as shown in SEQ ID NO: 120; the VH comprises a VH sequence as shown in SEQ ID NO: 121 and the VL comprises a VL sequence as shown in SEQ ID NO: 122; the VH comprises a VH sequence as shown in SEQ ID NO: 123 and the VL comprises a VL sequence as shown in SEQ ID NO: 124; the VH comprises a VH sequence as shown in SEQ ID NO: 125 and the VL comprises a VL sequence as shown in SEQ ID NO: 126; the VH comprises a VH sequence as shown in SEQ ID NO: 127 and the VL comprises a VL sequence as shown in SEQ ID NO: 128; the VH comprises a VH sequence as shown in SEQ ID NO: 133 and the VL comprises a VL sequence as shown in SEQ ID NO: 134; or the VH comprises a VH sequence as shown in SEQ ID NO: 135 and the VL comprises a VL sequence as shown in SEQ ID NO: 136.

In embodiments provided herein involving an antibody with conditional affinity which specifically binds an antigen with higher affinity under a low agent concentration condition than under a high agent concentration condition, optionally, the ratio of: [K_{D} of the antibody-antigen binding at 25° C. at high agent concentration] / [K_{D} of the antibody-antigen binding at 25° C. at low agent concentration] is more than, or ranges between 2, 3, 4, 8, 10, 16 or more. Optionally, the ratio of: [k_{off} of the antibody-antigen binding at 25° C. at high agent concentration] / [k_{off} of the antibody-antigen binding at 25° C. at low agent concentration] is more than, or ranges between 2, 3, 4, 8, 10, 16 or more. Optionally, the ratio of: [K_{D} of the antibody-antigen binding at 25° C. at high agent concentration] / [K_{D} of the antibody-antigen binding at 25° C. at low agent concentration] is more than, or ranges between 20, 30, 40 or 100 or more. Optionally, the ratio of: [k_{off} of the antibody-antigen binding at 25° C. at high agent concentration] / [k_{off} of the antibody-antigen binding at 25° C. at low agent concentration] is more than, or ranges between 20, 30, 40 or 100 or more. Optionally, the binding of the antibody to the antigen under a low agent concentration condition at 25° C. has a K_{D} of about 0.01 nM to about 100 nM. Optionally, the binding of the antibody to the antigen under a low agent concentration condition at 25° C. has a K_{D} of about 0.1 nM to about 10 nM. Optionally, the binding of the antibody to the antigen under a low agent concentration condition at 25° C. has a K_{D} of about 1 nM to about 500 nM. Optionally, the binding of the antibody to the antigen under a low agent concentration condition at 25° C. has a K_{D} of about 50 nM to about 250 nM. Optionally, the binding of the antibody to the antigen under a low agent concentration condition at 25° C. has a k_{off} of about 1×10 E⁻⁴ S⁻¹ to about 1×10 E⁻¹ S⁻¹. Optionally, the binding of the antibody to the antigen under a low agent concentration condition at 25° C. has a k_{off} of about 1×10 E⁻³ S⁻¹ to about 1×10 E⁻¹ S⁻¹. Optionally, the binding of the antibody to the antigen at pH 7.4, 25° C., and 0 µM of small molecule agent has a K_{D} of about y to about 10 nM, whereas the binding of the same antibody to the same antigen at pH 7.4, 25° C., and 10 µM or greater concentration of small molecule agent has a K_{D} of about 100 nM to about 1 µM. Optionally, the K_{D} of the binding of the antibody to the antigen at pH 7.4, 25° C. and 10 µM concentration of small molecule agent is at least 2, 3, 4, 5, 10, 15, or 20 times larger than the K_{D} of the binding of the same antibody to the same antigen at pH 7.4, 25° C. and 0 µM of the small molecule agent.

In embodiments provided herein involving an antibody with conditional affinity which specifically binds an antigen with higher affinity under a high agent concentration condition than under a low agent concentration condition, optionally, the ratio of: [K_{D} of the antibody-antigen binding at 25° C. at low agent concentration] / [K_{D} of the antibody-antigen binding at 25° C. at high agent concentration] is more than, or ranges between 2, 3, 4, 8, 10, 16 or more. Optionally, the ratio of: [k_{off} of the antibody-antigen binding at 25° C. at low agent concentration] / [k_{off} of the antibody-antigen binding at 25° C. at high agent concentration] is more than, or ranges between 2, 3, 4, 8, 10, 16 or more. Optionally, the ratio of: [K_{D} of the antibody-antigen binding at 25° C. at low agent concentration] / [K_{D} of the antibody-antigen binding at 25° C. at high agent concentration] is more than, or ranges between 20, 30, 40 or 100 or more. Optionally, the ratio of: [k_{off} of the antibody-antigen binding at 25° C. at low agent concentration] / [k_{off} of the antibody-antigen binding at 25° C. at high agent concentration] is more than, or ranges between 20, 30, 40 or 100 or more. Optionally, the binding of the antibody to the antigen under a high agent concentration condition at 25° C. has a K_{D} of about 0.01 nM to about 100 nM. Optionally, the binding of the antibody to the antigen under a high agent concentration condition at 25° C. has a K_{D} of about 0.1 nM to about 10 nM. Optionally, the binding of the antibody to the antigen under a high agent concentration condition at 25° C. has a K_{D} of about 1 nM to about 500 nM. Optionally, the binding of the antibody to the antigen under a high agent concentration condition at 25° C. has a K_{D} of about 50 nM to about 250 nM. Optionally, the binding of the antibody to the antigen under a high agent concentration condition at 25° C. has a k_{off} of about 1×10 E⁻⁴ S⁻¹ to about 1×10 E-¹ S⁻¹. Optionally, the binding of the antibody to the antigen under a high agent concentration condition at 25° C. has a k_{off} of about 1×10 E⁻³ S⁻¹ to about 1×10 E⁻¹ S⁻¹. Optionally, the binding of the antibody to the antigen at pH 7.4, 25° C., and 10 µM or greater concentration of small molecule agent has a K_{D} of about 0.1 nM to about 10 nM, whereas the binding of the same antibody to the same antigen at pH 7.4, 25° C., and 0 µM concentration of small molecule agent has a K_{D} of about 100 nM to about 1 µM. Optionally, the K_{D} of the binding of the antibody to the antigen at pH 7.4, 25° C. and 0 µM concentration of small molecule agent is at least 2, 3, 4, 5, 10, 15, or 20 times larger than the K_{D} of the binding of the same antibody to the same antigen at pH 7.4, 25° C. and 10 µM of the small molecule agent.

In some embodiments, an antibody with conditional affinity provided herein that contains a VH and VL contains an agent-binding motif within the VH or VL region. In some embodiments, the agent-binding motif is within the VH. In some embodiments, the agent-binding motif is within the VL. In some embodiments, an agent-binding motif comprises at least one of VH CDR1, VH CDR2, or VH CDR3. In some embodiments, an agent-binding motif comprises VH CDR1 and VH CDR2. In some embodiments, an agent-binding motif comprises VH CDR1, VH CDR2, and VH CDR3. In some embodiments, an agent-binding motif comprises at least one of VL CDR1, VL CDR2, or VL CDR3. In some embodiments, an agent-binding motif comprises VL CDR1 and VL CDR2. In some embodiments, an agent-binding motif comprises VL CDR1, VL CDR2, and VL CDR3.

In embodiments provided herein involving a low agent concentration and a high agent concentration, optionally, the low agent concentration is about 0 nM to about 0.1 nM, and the high agent concentration is about 1 nM to about 500 nM. Optionally, the low agent concentration is about 0 nM to about 0.1 nM, and the high agent concentration is about 1 nM to about 500 µM. Optionally, the low agent concentration is about 0 nM to about 10 nM, and the high agent concentration is about 50 nM to about 500 nM. Optionally, the low agent concentration is about 0 nM to about 1 nM, and the high agent concentration is about 100 nM to about 100 mM. Optionally, the low agent concentration is about 0 nM to about 1 µM, and the high agent concentration is about 100 µM to about 10 mM. Optionally, the high agent concentration is at least about 2-times, 3-times, 5-times, 10-times, 20-times, 50-times, 100-times, or 1000-times greater than the low agent concentration. Optionally, the low agent concentration is a serum concentration of agent. Optionally, the high agent concentration is a serum concentration of agent.

In embodiments provided herein involving an antibody with conditional affinity containing an agent-binding motif, optionally, the agent-binding motif is located within VH CDR1 and VH CDR2 of the antibody.

In embodiments provided herein involving a small molecule agent, optionally, the agent is folic acid, an inhibitor of dihydrofolate reductase (DHFR), sulfasalazine, hydroxychloroquine, leflunomide, imatinib, gefitinib, erlotinib, sorafenib, abiraterone, crizotinib, vemurafenib, vismodegib, sonidegib, everolimus, tamoxifen, toremifene, fulvestrant, anastrozole, exemestane, lapatinib, letrozole, emtansine, palbociclib, ziv-aflibercept, regorafenib, imatinib mesylate, lanreotide acetate, sunitinib, alitretinoin, pazopanib, temsirolimus, axitinib, tertinoin, dasatinib, nilotinib, bosutinib, ibrutinib, idelalisib, gefitinib, afatinib dimaleate, ceritinib, denileukin diftitox, vorinostat, romidepsin, bexarotene, bortezomib, pralatrexate, lenalimodie, belinostat, vemurafenib, trametinib, dabrafenib, carfilzomib, lenaliomide, pomalidomide, panobinostat, ruxolitinib phosphate, cabozantinib, or lenvatinib mesylate. Optionally, an inhibitor of DHFR is methotrexate, aminopterin, or a variant thereof.

Optionally, a small molecule agent provided herein is methotrexate.

In some embodiments, a small molecule agent may naturally occur in a subject, such that a high concentration condition of the small molecule agent constitutively or periodically occurs in a subject, such that an antibody with conditional affinity is in a high concentration condition of an agent in a subject, even if the subject is not separately administered the small molecule agent.

In some embodiments, a small molecule agent may occur in a high concentration condition in a localized environment in a subject (e.g. in a tissue microenvironment, such as a tumor microenvironment), such that an antibody with conditional affinity in a subject is in a high concentration condition of an agent in a particular environment in the subject. In this way, an antibody with conditional affinity may bind to an antigen with higher affinity or lower affinity in a particular environment in a subject (depending, for example, on the concentration of the agent in the particular environment and on the effect of the small molecule agent on the affinity of the antibody for the antigen) than in other locations / environments in the subject.

In some embodiments, a small molecule agent does not naturally occur in a subject or humans.

In some embodiments, provided herein is a library comprising polynucleotides encoding a heterogeneous population of antibodies, wherein at least one of the antibodies specifically binds an antigen with higher affinity under a low agent concentration condition than under a high agent concentration condition, wherein the antibody comprises a heavy chain variable region (VH) and a light chain variable region (VL); the antibody comprises an agent-binding motif within the VH or VL; and the agent is a small molecule.

In some embodiments, provided herein is a library comprising polynucleotides encoding a heterogeneous population of antibodies, wherein at least one of the antibodies specifically binds an antigen with higher affinity under a high agent concentration condition than under a low agent concentration condition, wherein the antibody comprises a heavy chain variable region (VH) and a light chain variable region (VL); the antibody comprises an agent-binding motif within the VH or VL; and the agent is a small molecule.

In some embodiments, provided herein is a library comprising polynucleotides encoding a heterogeneous population of antibodies, wherein at least one of the antibodies specifically binds an antigen with higher affinity under a low agent concentration condition than under a high agent concentration condition, wherein the antibody comprises a heavy chain variable region (VH) and a light chain variable region (VL); and the agent is a small molecule.

In some embodiments, provided herein is a library comprising polynucleotides encoding a heterogeneous population of antibodies, wherein at least one of the antibodies specifically binds an antigen with higher affinity under a high agent concentration condition than under a low agent concentration condition, wherein the antibody comprises a heavy chain variable region (VH) and a light chain variable region (VL); and the agent is a small molecule.

In some embodiments, provided herein is a library comprising polynucleotides encoding a heterogeneous population of antibodies, wherein at least a first polynucleotide of the polynucleotides encodes a heavy chain variable region (VH) of an antibody that specifically binds an antigen with higher affinity under a low agent concentration than under a high agent concentration, wherein: a) the antibody comprises a VH and a light chain variable region (VL); and b) the agent is a small molecule. Optionally, the low agent concentration is about 0 nM to about 1 nM, and the high agent concentration is about 5 nM to about 100 mM. Optionally, the first polynucleotide encodes a VH region that comprises a CDR1 and a CDR2 of the partial VH sequence shown in SEQ ID NO: 1. Optionally, the first polynucleotide encodes a VH region that comprises a CDR1 comprising an amino acid sequence as shown in SEQ ID NO: 43, 116, or 117 and a CDR2 comprising an amino acid sequence as shown in SEQ ID NO: 44 or 118. Optionally, the first polynucleotide encodes a VH region that comprises a framework region 3 (FR3) comprising the amino acid sequence Cys-Ala-Ala. Optionally, the first polynucleotide encodes a VH region that comprises an amino acid sequence as shown in SEQ ID NO: 1. Optionally, the first polynucleotide encodes a VH region that comprises an amino acid sequence as shown in SEQ ID NO: 166. Optionally, a second polynucleotide of the polynucleotides encodes a VH region that comprises a CDR1 and a CDR2 of the partial VH sequence shown in SEQ ID NO: 1. Optionally, the second polynucleotide encodes a VH region that comprises an amino acid sequence as shown in SEQ ID NO: 1. Optionally, the second polynucleotide encodes a VH region that comprises an amino acid sequence as shown in SEQ ID NO: 166. Optionally, the first polynucleotide and the second polynucleotide each encode a VH region comprising a CDR3 sequence, and wherein VH CDR3 sequence encoded by the first polynucleotide is different from the VH CDR3 sequence encoded by the second polynucleotide. Optionally, the agent is methotrexate.

In some embodiments, provided herein is a library comprising polynucleotides encoding a heterogeneous population of antibodies, wherein at least a first polynucleotide of the polynucleotides encodes a heavy chain variable region (VH) of an antibody that specifically binds an antigen with higher affinity under a high agent concentration than under a low agent concentration, wherein: a) the antibody comprises a VH and a light chain variable region (VL); and b) the agent is a small molecule. Optionally, the low agent concentration is about 0 nM to about 1 nM, and the high agent concentration is about 5 nM to about 100 mM. Optionally, the first polynucleotide encodes a VH region that comprises a CDR1 and a CDR2 of the partial VH sequence shown in SEQ ID NO: 1. Optionally, the first polynucleotide encodes a VH region that comprises a CDR1 comprising an amino acid sequence as shown in SEQ ID NO: 43, 116, or 117 and a CDR2 comprising an amino acid sequence as shown in SEQ ID NO: 44 or 118. Optionally, the first polynucleotide encodes a VH region that comprises a framework region 3 (FR3) comprising the amino acid sequence Cys-Ala-Ala. Optionally, the first polynucleotide encodes a VH region that comprises an amino acid sequence as shown in SEQ ID NO: 1. Optionally, the first polynucleotide encodes a VH region that comprises an amino acid sequence as shown in SEQ ID NO: 166. Optionally, a second polynucleotide of the polynucleotides encodes a VH region that comprises a CDR1 and a CDR2 of the partial VH sequence shown in SEQ ID NO: 1. Optionally, the second polynucleotide encodes a VH region that comprises an amino acid sequence as shown in SEQ ID NO: 1. Optionally, the second polynucleotide encodes a VH region that comprises an amino acid sequence as shown in SEQ ID NO: 166. Optionally, the first polynucleotide and the second polynucleotide each encode a VH region comprising a CDR3 sequence, and wherein VH CDR3 sequence encoded by the first polynucleotide is different from the VH CDR3 sequence encoded by the second polynucleotide. Optionally, the agent is methotrexate.

In some embodiments, provided herein is an isolated nucleic acid that encodes at least one or more VH or VL CDRs, the VH region, the VL region, or both the VH region and VL region of any of the antibodies provided herein. Also provided herein is an isolated nucleic acid of a library provided herein.

In some embodiments, provided herein is an isolated nucleic acid encoding a polypeptide comprising: a single-chain Fv domain (scFv) which comprises a heavy chain variable region (VH) and a light chain variable region (VL), and which specifically binds an antigen with higher affinity under a low agent concentration condition than under a high agent concentration condition; a transmembrane domain; and an intracellular signaling domain; and wherein the agent is a small molecule. Optionally, the scFv comprises an agent-binding motif within the VH or VL.

In some embodiments, provided herein is an isolated nucleic acid encoding a polypeptide comprising: a single-chain Fv domain (scFv) which comprises a heavy chain variable region (VH) and a light chain variable region (VL), and which specifically binds an antigen with higher affinity under a high agent concentration condition than under a low agent concentration condition; a transmembrane domain; and an intracellular signaling domain; and wherein the agent is a small molecule. Optionally, the scFv comprises an agent-binding motif within the VH or VL.

In embodiments provided herein involving a nucleic acid encoding a polypeptide comprising a single-chain Fv domain (scFv) which comprises a heavy chain variable region (VH) and a light chain variable region (VL); a transmembrane domain; and an intracellular signaling domain, the scFv may have any of the characteristics of an antibody with conditional affinity provided herein. Optionally, the transmembrane domain comprises a transmembrane domain of CD8, CD28, 4-1 BB, CD3, CD4, CD8, [symbol] NKG2D, Fcε [symbol] ICOS, CTLA-4, PD-1, or VISTA. Optionally, the intracellular signaling domain comprises a signaling domain of CD3, 4-1 BB, CD28, ICOS, 4-1 BB, OX40 or an inhibitory signaling domain of CTLA-4, PD-1, or VISTA. Optionally, the intracellular signaling domain comprises a CD3ζ signaling domain and a 4-1BB signaling domain.

In embodiments provided herein involving a nucleic acid encoding a polypeptide comprising a single-chain Fv domain (scFv) which comprises a heavy chain variable region (VH) and a light chain variable region (VL); a transmembrane domain; and an intracellular signaling domain, optionally, the VH region comprises a CDR1 and a CDR2 of the partial VH sequence shown in SEQ ID NO: 1. Optionally, the VH region comprises a CDR1 comprising an amino acid sequence as shown in SEQ ID NO: 43, 116, or 117 and a CDR2 comprising an amino acid sequence as shown in SEQ ID NO: 44 or 118. Optionally, the VH region comprises a framework region 3 (FR3) comprising the amino acid sequence Cys-Ala-Ala. Optionally, the VH region comprises an amino acid sequence as shown in SEQ ID NO: 1. Optionally, the VH region comprises an amino acid sequence as shown in SEQ ID NO: 166. Optionally, the VH region comprises a VH CDR1, VH CDR2, and VH CDR3 of a VH sequence of a clone provided in Table 3 or Table 6 herein. Optionally, the VH region comprises a VH sequence of a clone provided in Table 3 or Table 6 herein. Optionally, the VH region comprises a CDR1, CDR2, and CDR3 of a VH sequence as shown in any of SEQ ID NOs: 3, 7, 9, 13, 15, 17, 19, 21, 23, 25, 27, 29, 31, 33, 35, 37, 39, 41, 119, 121, 123, 125, 127, 133, or 135. Optionally, the VH region comprises a VH sequence as shown in any of SEQ ID NOs: 3, 7, 9, 13, 15, 17, 19, 21, 23, 25, 27, 29, 31, 33, 35, 37, 39, 41, 119, 121, 123, 125, 127, 133, or 135. Optionally, the antibody comprises a VH CDR1, VH CDR2, and VH CDR3 of a VH sequence of a clone provided in Table 3 or Table 6 herein, and a VL CDR1, VL CDR2, and VL CDR3 of a VL sequence of the same respective clone provided in Table 3 or Table 6. Optionally, the antibody comprises a VH sequence of a clone provided in Table 3 or Table 6 herein, and a VL sequence of the same respective clone provided in Table 3 or Table 6.

Also provided herein is a polypeptide encoded by any of the isolated nucleic acids described herein, wherein the polypeptide comprises a single-chain Fv domain (scFv) which comprises a heavy chain variable region (VH) and a light chain variable region (VL); a transmembrane domain; and an intracellular signaling domain.

In some embodiments, provided herein is an antibody with conditional antigen-binding affinity, wherein: a) the antibody can specifically bind both i) an antigen, and ii) a small molecule agent; b) the antibody can specifically bind at least i) the antigen alone or ii) the antigen and small molecule simultaneously; and c) the antibody binds to the antigen with a higher affinity when the antibody is bound to the antigen alone than when the antibody is bound to the antigen and small molecule simultaneously. Optionally, the antibody comprises a heavy chain variable region (VH) and a light chain variable region (VL), wherein antibody specifically binds the small molecule in the VH region. Optionally, the ratio of: [K_{D} of antibody-antigen interaction at a high agent concentration condition] to [K_{D} of antibody-antigen interaction at a low agent concentration condition] is more than or ranges between 2, 3, 4, 5, 8, 10, 16, 20, 30, 40, or 100 or more. Optionally, the ratio of: [K_{D} of antibody-antigen interaction at a high agent concentration condition] to [K_{D} of antibody-antigen interaction at a low agent concentration condition] is at least 1.5, 2, 3, 4, 5, 6, 7, 8, 9, 10, 15, 16, 20, 25, 30, 40, 50, 100, 200, 300, 400, 500, or 1000, or ranges between any of 1.5, 2, 3, 4, 5, 6, 7, 8, 9, 10, 15, 20, 25, 30, 40, 50, 100, 200, 300, 400, or 500 and any of 2, 3, 4, 5, 6, 7, 8, 9, 10, 15, 20, 25, 30, 40, 50, 100, 200, 300, 400, 500, or 1000, in which the second value is larger than the first value.

In some embodiments, provided herein is an antibody with conditional antigen-binding affinity, wherein: a) the antibody can specifically bind both i) an antigen, and ii) a small molecule agent; b) the antibody can specifically bind at least i) the antigen alone or ii) the antigen and the small molecule simultaneously; and c) the antibody specifically binds to the antigen with a higher affinity when it is bound to the antigen and the small molecule simultaneously than when it is bound to the antigen alone. Optionally, the antibody comprises a heavy chain variable region (VH) and a light chain variable region (VL), wherein antibody specifically binds the small molecule in the VH region. Optionally, the ratio of: [K_{D} of antibody-antigen interaction at a low agent concentration condition] to [K_{D} of antibody-antigen interaction at a high agent concentration condition] is more than or ranges between 2, 3, 4, 5, 8, 10, 16, 20, 30, 40, or 100 or more. Optionally, the ratio of: [K_{D} of antibody-antigen interaction at a low agent concentration condition] to [K_{D} of antibody-antigen interaction at a high agent concentration condition] is at least 1.5, 2, 3, 4, 5, 6, 7, 8, 9, 10, 15, 16, 20, 25, 30, 40, 50, 100, 200, 300, 400, 500, or 1000, or ranges between any of 1.5, 2, 3, 4, 5, 6, 7, 8, 9, 10, 15, 20, 25, 30, 40, 50, 100, 200, 300, 400, or 500 and any of 2, 3, 4, 5, 6, 7, 8, 9, 10, 15, 20, 25, 30, 40, 50, 100, 200, 300, 400, 500, or 1000, in which the second value is larger than the first value.

In some embodiments, provided herein is an antibody with conditional antigen-binding affinity, wherein: a) the antibody comprises a heavy chain variable region (VH) and a light chain variable region (VL); b) the antibody can specifically bind both i) an antigen, and ii) a small molecule agent; c) the antibody can specifically bind i) the antigen alone, ii) the small molecule agent alone, or iii) the antigen and small molecule simultaneously; and d) the antibody binds to the antigen with a higher affinity when the antibody is bound to the antigen alone than when the antibody is bound to the antigen and small molecule agent simultaneously. Optionally, the antigen binds to a first location on the antibody and the small molecule agent binds to a second location on the antibody. Optionally, the first location on the antibody and the second location on the antibody do not overlap. Optionally, the first location on the antibody and the second location on the antibody at least partially overlap. Optionally, the antibody specifically binds the small molecule agent in the VH region.

In some embodiments, provided herein is an antibody with conditional antigen-binding affinity, wherein: a) the antibody comprises a heavy chain variable region (VH) and a light chain variable region (VL); b) the antibody can specifically bind both i) an antigen, and ii) a small molecule agent; c) the antibody can specifically bind i) the antigen alone, ii) the small molecule agent alone, or iii) the antigen and small molecule simultaneously; and d) the antibody binds to the antigen with a lower affinity when the antibody is bound to the antigen alone than when the antibody is bound to the antigen and small molecule agent simultaneously. Optionally, the antigen binds to a first location on the antibody and the small molecule agent binds to a second location on the antibody. Optionally, the first location on the antibody and the second location on the antibody do not overlap. Optionally, the first location on the antibody and the second location on the antibody at least partially overlap. Optionally, the antibody specifically binds the small molecule agent in the VH region.

In some embodiments, in an antibody having conditional affinity provided herein which comprises a VH region and for which the affinity of the antibody is modulated by the concentration of a small molecule agent and/or which specifically binds to a small molecule agent, the VH region comprises a CDR1 and a CDR2 of the partial VH sequence shown in SEQ ID NO: 1. Optionally, the small molecule agent specifically binds to the antibody at a location comprising a CDR1 and a CDR2 of the partial VH sequence shown in SEQ ID NO: 1. Optionally, the VH region comprises a CDR1 comprising an amino acid sequence as shown in SEQ ID NO: 43, 116, or 117 and a CDR2 comprising an amino acid sequence as shown in SEQ ID NO: 44 or 118. Optionally, the small molecule agent specifically binds to the antibody at a location comprising a CDR1 comprising an amino acid sequence as shown in SEQ ID NO: 43, 116, or 117 and a CDR2 comprising an amino acid sequence as shown in SEQ ID NO: 44 or 118. Optionally, the VH region comprises a framework region 3 (FR3) comprising the amino acid sequence Cys-Ala-Ala. Optionally, the small molecule agent specifically binds to the antibody at a location comprising a VH FR3 comprising the amino acid sequence Cys-Ala-Ala. Optionally, the VH region comprises an amino acid sequence as shown in SEQ ID NO: 1. Optionally, the small molecule agent specifically binds to the antibody at a location comprising a VH region comprising an amino acid sequence as shown in SEQ ID NO: 1. Optionally, the VH region comprises a CDR1, CDR2, and CDR3 of a VH sequence as shown in any of SEQ ID NOs: 3, 7, 9, 13, 15, 17, 19, 21, 23, 25, 27, 29, 31, 33, 35, 37, 39, 41, 119, 121, 123, 125, 127, 133, or 135. Optionally, the VH region comprises a VH sequence as shown in any of SEQ ID NOs: 3, 7, 9, 13, 15, 17, 19, 21, 23, 25, 27, 29, 31, 33, 35, 37, 39, 41, 119, 121, 123, 125, 127, 133, or 135. Optionally, the VH region comprises a VH sequence as shown in SEQ ID NO: 166.

In some embodiments, provided herein is an isolated cell line, host cell, or hybridoma that produces or contains an antibody provided herein.

In some embodiments, provided herein is a recombinant expression vector comprising an isolated nucleic acid provided herein. Also provided is a host cell comprising an expression vector provided herein. Optionally, the heavy and light chains of the antibody are encoded on separate vectors. Optionally, the heavy and light chains of the antibody are encoded on the same vector.

In some embodiments, provided herein is a method of producing an antibody with conditional affinity, the method comprising: culturing a cell line that recombinantly produces an antibody with conditional affinity provided herein, wherein the antibody is produced; and recovering the antibody.

In some embodiments, provided herein is a pharmaceutical composition comprising an antibody with conditional affinity provided herein, and a pharmaceutically acceptable carrier. Also provided is a kit comprising a pharmaceutical composition provided herein.

In some embodiments, provided herein is a lymphocyte comprising a polypeptide encoded by a nucleic acid provided herein, such as a polypeptide comprising: a single-chain Fv domain (scFv) which comprises a heavy chain variable region (VH) and a light chain variable region (VL), and which specifically binds an antigen with higher affinity under a low agent concentration condition than under a high agent concentration condition; a transmembrane domain; and an intracellular signaling domain; and the agent is a small molecule. Optionally, the scFv comprises an agent-binding motif within the VH or VL. Optionally, the lymphocyte is a T cell.

In some embodiments, provided herein is a lymphocyte comprising a polypeptide encoded by a nucleic acid provided herein, such as a polypeptide comprising: a single-chain Fv domain (scFv) which comprises a heavy chain variable region (VH) and a light chain variable region (VL), and which specifically binds an antigen with higher affinity under a high agent concentration condition than under a low agent concentration condition; a transmembrane domain; and an intracellular signaling domain; and the agent is a small molecule. Optionally, the scFv comprises an agent-binding motif within the VH or VL. Optionally, the lymphocyte is a T cell.

In some embodiments, provided herein is a lymphocyte comprising a chimeric antigen receptor (CAR) comprising a scFv which comprises a heavy chain variable region (VH) and a light chain variable region (VL), wherein the scFv has any properties of an antibody with conditional affinity provided herein. Optionally, the lymphocyte is a T cell.

In some embodiments, provided herein is a method of treating a disorder in a subject, the method comprising administering to a subject in need thereof an antibody having conditional affinity or a lymphocyte comprising a chimeric antigen receptor (CAR) comprising a scFv which comprises a heavy chain variable region (VH) and a light chain variable region (VL), wherein the scFv has any properties of an antibody with conditional affinity provided herein. Optionally, the method further comprises administering a small molecule agent to the subject, wherein the small molecule agent affects the affinity of the antibody or scFv of the polypeptide for an antigen in the subject. Optionally, the small molecule agent may be administered before, at about the same time, or after the antibody or lymphocyte is administered to the subject.

In some embodiments, provided herein is a method of treating a disorder in a subject, the method comprising: a) at a first time, administering to the subject an effective amount of an antibody with conditional affinity as provided herein, wherein the antibody specifically binds an antigen with higher affinity under a low agent concentration condition than under a high agent concentration condition, and b) at a second time, administering to the subject a small molecule agent, wherein the agent reduces the affinity of the antibody for the antigen. Optionally, the administering to the subject the small molecule agent reduces a physiological effect in the subject that results from the antibody specifically binding to the antigen.

In some embodiments, provided herein is a method of treating a disorder in a subject, the method comprising: a) at a first time, administering to the subject an effective amount of a lymphocyte provided herein, wherein the lymphocyte comprises a polypeptide encoded by a nucleic acid provided herein, wherein the polypeptide comprises: a single-chain Fv domain (scFv) which comprises a heavy chain variable region (VH) and a light chain variable region (VL), and which specifically binds an antigen with higher affinity under a low agent concentration condition than under a high agent concentration condition; a transmembrane domain; and an intracellular signaling domain; and the agent is a small molecule, and b) at a second time, administering to the subject a small molecule agent, wherein the small molecule agent reduces the affinity of the scFv for the antigen. Optionally, the administering to the subject of the small molecule agent reduces a physiological effect in the subject that results from the scFv specifically binding to the antigen.

In some embodiments, provided herein is a method of treating a disorder in a subject, the method comprising: administering to the subject i) an effective amount of an antibody with conditional affinity provided herein, wherein the antibody specifically binds an antigen with higher affinity under a high agent concentration condition than under a low agent concentration condition and ii) a small molecule agent that specifically binds to the antibody. Optionally, the antibody is administered to the subject before the small molecule agent is administered to the subject.

In some embodiments, provided herein is a method of treating a disorder in a subject, the method comprising: administering to the subject i) an effective amount of a lymphocyte provided herein, wherein the lymphocyte comprises a polypeptide encoded by a nucleic acid provided herein, wherein the polypeptide comprises: a single-chain Fv domain (scFv) which comprises a heavy chain variable region (VH) and a light chain variable region (VL), and which specifically binds an antigen with higher affinity under a high agent concentration condition than under a low agent concentration condition; a transmembrane domain; and an intracellular signaling domain, wherein the scFv comprises an agent-binding motif within the VH or VL; and the agent is a small molecule, and ii) a small molecule agent that specifically binds to the scFv of the polypeptide of the lymphocyte. Optionally, the lymphocyte is administered to the subject before the small molecule agent is administered to the subject. Optionally, the lymphocyte is administered to the subject at about the same time or after the small molecule agent is administered to the subject.

In methods provided herein involving a first time and a second time, typically, the second time is at time point after the first time (e.g. the second time may be, for example, 1, 2, 4, 6, 12, or 24 hours after the first time).

In some embodiments, the affinity of an antibody provided herein for an antigen increases when the antibody is in a high concentration of a small molecule agent, as compared to when the antibody is in a low concentration of the small molecule agent. In such embodiments, the binding of the small molecule agent to the antibody increases the affinity of the antibody for the antigen. For example, in some embodiments, an antibody provided herein has conditional affinity for its antigen such that the affinity of the antibody for its antigen at 10 micromolar or greater concentration of the small molecule agent is more than at 0 micromolar concentration of the small molecule agent. In other words, in some examples, the K_{D} or k_{off} ratio at 0 micromolar small molecule agent to 10 micromolar small molecule agent (i.e. ratio of: antibody-antigen K_{D} or k_{off} at 0 micromolar small molecule agent / antibody-antigen K_{D} or k_{off} at 10 micromolar small molecule agent) is more than, or ranges between, 2, 3, 4, 8, 10, 16, 20, 30, 40, 50, and/or 100.

In other embodiments, the affinity of an antibody provided herein for an antigen decreases when the antibody is in a high concentration of a small molecule agent, as compared to when the antibody is in a low concentration of the small molecule agent. In such embodiments, the binding of the small molecule agent to the antibody decreases the affinity of the antibody for the antigen. For example, in some embodiments, an antibody provided herein has conditional affinity for binding to its antigen such that the affinity of the antibody for its antigen at 0 micromolar concentration of the small molecule agent is more than at 10 micromolar or greater concentration of the small molecule agent. In other words, in examples, the K_{D} or k_{off} ratio at 10 micromolar small molecule agent to 0 micromolar small molecule agent (i.e. ratio of: antibody-antigen K_{D} or k_{off} at 10 micromolar small molecule agent / antibody-antigen K_{D} or k_{off} at 0 micromolar small molecule agent) is more than, or ranges between, 2, 3, 4, 8, 10, 16, 20, 30, 40, 50, and/or 100.

In some embodiments, K_{D} or k_{off} may be measured at 25° C. or 37° C. Similarly, any embodiment provided herein involving performing a measurement at 25° C. may alternatively comprise performing the measurement at 37° C. or other suitable temperature.

In some embodiments, a k_{off} may be determined for any value between about 1×10 E⁻⁵ s⁻¹ to about 1 s⁻¹.

In some embodiments, an antigen of an antibody provided herein may be BCMA, TIM3, CD19, CD33, EGFR, HER2, TNF alpha, TNF beta (lymphotoxin-alpha), or CD123.

In some embodiments, an antibody with conditional affinity provided herein may contain a VH region and a VL region, it may bind to a small molecule agent at a location in the VH region of the antibody, and it may bind to an antigen at a location formed by the combination of the VH region and the VL region (i.e. the antigen-binding location may be at the interface between the VH and VL regions).

In some embodiments, provided herein is a method of generating an antibody with conditional affinity, the method comprising A) obtaining a genetic library encoding multiple Fab clones, wherein the Fabs each comprise: i) a heavy chain encoding at least one of a CDR1, CDR2, or CDR3 from a VHH antibody which specifically binds to a small molecule agent of interest, and ii) a light chain; B) screening the library for clones which bind to an antigen of interest; C) screening at least some of the clones identified in B) for clones which have a different affinity for the antigen of interest depending on the concentration of the small molecule agent of interest. Optionally, the heavy chain encodes at least the CDR1 and CDR2 from a VHH antibody which specifically binds to the small molecule agent of interest.

In some embodiments, provided herein is an antibody with conditional affinity wherein the affinity of the antibody for its antigen is affected by the concentration of the small molecule agent methotrexate, wherein the last three amino acids of the VH FR3 region of the antibody are, in sequential order, cysteine-alanine-alanine.

In some embodiments, provided herein is an antibody with conditional affinity comprising a VH and VL region, wherein the VH and VL region collectively comprise at least 1, 2, 3, 4, 5, or all 6 CDRs of an antibody with conditional affinity provided herein.

Also provided herein are pharmaceutical compositions comprising a therapeutically effective amount of any of the antibodies with conditional affinity provided herein, a host cell that recombinantly produces any of the antibodies with conditional affinity provided herein, and an isolated nucleic acid encoding any of the antibodies with conditional affinity provided herein.

In some embodiments, an antibody with conditional affinity provided herein may be a human, humanized, or chimeric antibody. In some embodiments, the antibody is a monoclonal antibody. In some embodiments, the antibody comprises a constant region. In some embodiments, the antibody is of the human IgG₁, lgG₂, IgG_{2Δa}, IgG₃, IgG₄, IgG_{4Δb}, lgG_{4Δc}, IgG₄ S228P, IgG_{4Δb} S228P, and IgG_{4Δc} S228P subclass. In some embodiments, the antibody is of the IgG4 isotype and comprises a stabilized hinge, e.g., S228P.

In another aspect, provided herein is a method for treating a condition in a subject comprising administering to the subject in need thereof an effective amount of a pharmaceutical composition as described herein, and optionally, a small molecule agent. In some embodiments, the condition is a cancer. In some embodiments, the cancer is selected from the group consisting of gastric cancer, sarcoma, lymphoma, leukemia, head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, multiple myeloma, renal cell carcinoma, bladder cancer, cervical cancer, choriocarcinoma, colon cancer, oral cancer, skin cancer, and melanoma. In some embodiments, the subject is a previously treated adult patient with locally advanced or metastatic melanoma, squamous cell head and neck cancer (SCHNC), ovarian carcinoma, sarcoma, or relapsed or refractory classic Hodgkin’s Lymphoma (cHL). In some embodiments, the cancer can be a platinum resistant and/or platinum refractory cancer, such as, for example, platinum resistant and/or refractory ovarian cancer, platinum resistant and/or /refractory breast cancer, or platinum resistant and/or refractory lung cancer. In some embodiments, an antibody with conditional affinity provided herein is administered at a dosage of about 0.5 mg/kg, about 1.0 mg/kg, about 3.0 mg/kg, or about 10 mg/kg. In some embodiments, the antibody is administered once every 7, 14, 21, or 28 days. In some embodiments, the antibody is administered intravenously or subcutaneously.

Also provided herein is a method of inhibiting tumor growth or progression in a subject who has a tumor, comprising administering to the subject an effective amount of a pharmaceutical composition as described herein, and optionally, a small molecule agent.

In some embodiments, provided herein is a method of inhibiting or preventing metastasis of cancer cells in a subject, comprising administering to the subject in need thereof an effective amount of a pharmaceutical composition as described herein, and optionally, a small molecule agent.

In some embodiments, an antibody provided herein can be administered parenterally in a subject. In some embodiments, the subject is a human.

Also provided is the use of any of the antibodies with conditional affinity provided herein in the manufacture of a medicament. Optionally, the medicament is for the treatment of cancer or for inhibiting tumor growth or progression in a subject in need thereof.

Also provided are antibodies having conditional affinity for use in the treatment of a cancer or for inhibiting tumor growth or progression in a subject in need thereof. In some embodiments, the cancer is, for example without limitation, gastric cancer, sarcoma, lymphoma, Hodgkin’s lymphoma, leukemia, head and neck cancer, thymic cancer, epithelial cancer, salivary cancer, liver cancer, stomach cancer, thyroid cancer, lung cancer (including, for example, non-small-cell lung carcinoma), ovarian cancer, breast cancer, prostate cancer, esophageal cancer, pancreatic cancer, glioma, leukemia, multiple myeloma, renal cell carcinoma, bladder cancer, cervical cancer, choriocarcinoma, colon cancer, oral cancer, skin cancer, and melanoma.

In any embodiment provided herein involving an antibody having conditional affinity, optionally, the affinity of the antibody for an antigen may be measured in a surface plasmon-resonance-based biosensor at 25° C. or 37° C. in 10 mM Hepes pH 7.4, 150 mM NaCl, 0.05% Tween-20 (HBST+), and with or without 10 µM small molecule agent. Optionally, the small molecule agent is methotrexate.

Any of the above features of one embodiment, aspect, or option may be combined with a different feature described above of another embodiment, aspect, or option, as would be understood by a person of skill in the art and unless the context dictates otherwise.

### BRIEF DESCRIPTION OF THE DRAWINGS

FIG. 1 depicts schematics of exemplary antibodies with conditional affinity and conditions provided herein.

FIG. 2 depicts a schematic outlining various possible effects resulting from administration of methotrexate and a CAR T cell to a subject, in which the CAR of the CAR T cell contains an antibody (e.g. an scFv) with conditional affinity which has increased affinity for an antigen in a high methotrexate concentration condition as compared to a low methotrexate concentration condition.

FIG. 3 depicts a schematic outlining various possible effects resulting from administration of methotrexate and a CAR T cell to a subject, in which the CAR of the CAR T cell contains an antibody (e.g. a scFv) with conditional affinity which has increased affinity for an antigen in a low methotrexate concentration condition as compared to a high methotrexate concentration condition.

FIG. 4 shows a bar graph summarizing cytotoxicity of CAR T cells containing different anti-CD33 antibodies having conditional affinity, against target cells that express or do not express CD33.

FIG. 5 shows a bar graph summarizing cytotoxicity of CAR T cells containing different anti-CD33 antibodies having conditional affinity, against target cells that express CD33, in the presence or absence of MTX.

FIGS. 6A-6D show bar graphs that summarize the levels of secretion of the cytokines IL-2 (FIGS. 6A and 6B) and IFN-gamma (FIGS. 6C and 6D) by CAR T cells that contain an anti-CD33 antibody having conditional affinity, after two different methods of T cell stimulation, and in the presence or absence of MTX.

FIG. 7 shows a bar graph summarizing the amount of proliferation of CAR T cells that contain an anti-CD33 antibody having conditional affinity, after two different methods of T cell stimulation, and in the presence or absence of MTX.

FIG. 8 shows histograms that summarize the activation and proliferation of of CAR T cells that contain an anti-CD33 antibody having conditional affinity, in the presence or absence of MTX, and in the presence or absence of target cells, as assessed by flow cytometry.

FIGS. 9A and 9C show schematics of cytotoxicity assays using CAR T cells containing different anti-CD33 antibodies having conditional affinity, against target cells that express CD33, and in which the CAR T cells are incubated in the presence or absence of MTX during different rounds of the assays. FIGS. 9B and 9D show graphs summarizing cytotoxicity of CAR T cells in the assays outlined in the schematics of 9A and 9C, respectively.

FIG. 10 shows a bar graph summarizing cytotoxicity of CAR T cells containing different anti-EGFR antibodies having conditional affinity, against target cells that express EGFR, in the presence or absence of MTX.

FIG. 11 shows a bar graph summarizing cytotoxicity of CAR T cells containing different anti-CD33 antibodies having conditional affinity, against target cells that express CD33, in the presence or absence of MTX, or 2 different MTX analogues.

### DETAILED DESCRIPTION

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

The term “isolated molecule” as referring to a molecule (where the molecule is, for example, a polypeptide, a polynucleotide, or an antibody) that by virtue of its origin or source of derivation (1) is not associated with naturally associated components that accompany it in its native state, (2) is substantially free of other molecules from the same source, e.g., species, cell from which it is expressed, library, etc., (3) is expressed by a cell from a different species, or (4) does not occur in nature. Thus, a molecule that is chemically synthesized, or expressed in a cellular system different from the system from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

A “small molecule agent” refers to a small multi-atom molecule, generally of less than 3000 daltons. As used herein, a “small molecule agent” does not include single-atom ions, such as H⁺, Ca²⁺ or Mg²⁺ ions. As used herein, “small molecule agent” further includes small molecule agents as defined in the preceding two sentences, in which the small molecule agent has been chemically modified to improve one or more properties of the starting / original small molecule agent (e.g. to have increased solubility or stability in solution or a subject, increased half-life, etc.), and as a result of the modification, the mass of the small molecule agent exceeds 3000 daltons. For example, a small molecule agent may be covalently linked to another molecule (also referred to herein as a “conjugate molecule”) that improves one or more properties of the small molecule agent, and the combination of the small molecule agent plus the covalently linked molecule may have a mass that exceeds 3000 daltons. Thus, “small molecule agent” as used herein includes “small molecule agents” as defined in the first two sentences of this paragraph, in which the small molecule agent has been modified by, for example, PEGylation, and as a result of the modification, the “small molecule agent” has a mass exceeding 3000 daltons (e.g. due to the added PEG). In other words, a “small molecule agent” as provided herein can have a mass exceeding 3000 daltons, if that “small molecule agent” has an original / starting structure that can the affect the affinity of an antibody with conditional affinity provided herein for an antigen, and that original / starting structure has been modified (e.g. by addition of a conjugate molecule) to improve one or more properties of the small molecule agent in solution or in a subject. For example, a “small molecule agent” provided herein is methotrexate (454 daltons) (in some examples, unmodified methotrexate molecules can affect the affinity of antibodies with conditional affinity provided herein for an antigen); also included within the definition of “small molecule agent” is a methotrexate molecule that has been modified to improve one or more properties of the methotrexate, such as by PEGylation. Continuing with this example, PEGylated methotrexate is included within the definition of “small molecule agent” provided herein, even if the mass of PEGylated methotrexate exceeds 3000 daltons.

References herein to an “agent” refer to a “small molecule agent”, unless the context clearly dictates otherwise.

An “agent concentration condition” refers the concentration of a small molecule agent as provided herein. An “agent concentration condition” may be defined in, for example, mM, µM, or nM concentration of the small molecule agent, or any other suitable unit. References herein to the “concentration” of an agent may be used interchangeably with “concentration condition”.

“High” and “low” agent concentration conditions refer to relative concentrations between two or more conditions that are being compared, and do not refer to absolute concentration values (unless separately also defined with a value). For example, when a “high concentration condition” and a “low concentration condition” or the like are being compared in reference to the concentration of a small molecule agent provided herein, it is to be understood that there is a higher concentration of the small molecule agent in the “high concentration condition” than in the “low concentration condition”. However, the “high concentration condition” does not refer to a particular concentration value, and a “high concentration condition” of a first small molecule agent may reflect a different absolute concentration value than a “high concentration condition” of a second small molecule agent. Similarly, the difference between a “high concentration condition” and a “low concentration condition” for a particular small molecule agent may also vary with the antibody of interest.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also, unless otherwise specified, any antigen binding portion thereof that competes with the intact antibody for specific binding, fusion proteins comprising an antigen binding portion, and any other modified configuration of the immunoglobulin molecule that comprises an antigen recognition site. Antigen binding portions include, for example, Fab, Fab′, F(ab′)₂, Fd, Fv, domain antibodies (dAbs, e.g., shark and camelid antibodies), fragments including complementarity determining regions (CDRs), single chain variable fragment antibodies (scFv), maxibodies, minibodies, intrabodies, diabodies, triabodies, tetrabodies, v-NAR and bis-scFv, and polypeptides that contain at least a portion of an immunoglobulin that is sufficient to confer specific antigen binding to the polypeptide. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, lgA₁ and lgA₂. The heavy-chain constant regions that correspond to the different classes of immunoglobulins are called alpha, delta, epsilon, gamma, and mu, respectively. The subunit structures and three-dimensional configurations of different classes of immunoglobulins are well known.

References herein to an “antigen” of an antibody refer to an antigen to which the antibody specifically binds. An “antigen” of an antibody may also be referred to as a “cognate antigen” of the antibody, or the like.

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonical class as the subject variable region (Chothia and Lesk, J Mol Biol 196(4): 901-917, 1987).

In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the AbM definition, the contact definition, and the conformational definition.

The Kabat definition is a standard for numbering the residues in an antibody and is typically used to identify CDR regions. See, e.g., Johnson & Wu, 2000, Nucleic Acids Res., 28: 214-8. The Chothia definition is similar to the Kabat definition, but the Chothia definition takes into account positions of certain structural loop regions. See, e.g., Chothia et al., 1986, J. Mol. Biol., 196: 901-17; Chothia et al., 1989, Nature, 342: 877-83. The AbM definition uses an integrated suite of computer programs produced by Oxford Molecular Group that model antibody structure. See, e.g., Martin et al., 1989, Proc Natl Acad Sci (USA), 86:9268-9272; “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999, “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

As known in the art, a “constant region” of an antibody refers to the constant region of the antibody light chain or the constant region of the antibody heavy chain, either alone or in combination.

As used herein, “monoclonal antibody” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example. As used herein, “humanized” antibody refers to forms of non-human (e.g. murine) antibodies that are chimeric immunoglobulins, immunoglobulin chains, or fragments thereof (such as Fv, Fab, Fab′, F(ab′)₂ or other antigen-binding subsequences of antibodies) that contain minimal sequence derived from non-human immunoglobulin. Preferably, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. The humanized antibody may comprise residues that are found neither in the recipient antibody nor in the imported CDR or framework sequences, but are included to further refine and optimize antibody performance.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen binding residues.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

The term “epitope” refers to that portion of a molecule / antigen capable of being recognized by and bound by an antibody at one or more of the antibody’s antigen-binding regions. Epitopes often consist of a surface grouping of molecules such as amino acids or sugar side chains and have specific three-dimensional structural characteristics as well as specific charge characteristics. In some embodiments, the epitope can be a protein epitope. Protein epitopes can be linear or conformational. In a linear epitope, all of the points of interaction between the protein and the interacting molecule (such as an antibody) occur linearly along the primary amino acid sequence of the protein. A “nonlinear epitope” or “conformational epitope” comprises noncontiguous polypeptides (or amino acids) within the antigenic protein to which an antibody specific to the epitope binds. The term “antigenic epitope” as used herein, is defined as a portion of an antigen to which an antibody can specifically bind as determined by any method well known in the art, for example, by conventional immunoassays. Once a desired epitope on an antigen is determined, it is possible to generate antibodies to that epitope, e.g., using the techniques described in the present specification. Alternatively, during the discovery process, the generation and characterization of antibodies may elucidate information about desirable epitopes. From this information, it is then possible to competitively screen antibodies for binding to the same epitope. An approach to achieve this is to conduct competition and cross-competition studies to find antibodies that compete or cross-compete with one another for binding to an antigen.

The term “agonist” refers to a substance which promotes (i.e., induces, causes, enhances, or increases) the biological activity or effect of another molecule. The term agonist encompasses substances which bind receptors, such as an antibody, and substances which promote receptor function without binding thereto (e.g., by activating an associated protein).

The term “antagonist” or “inhibitor” refers to a substance that prevents, blocks, inhibits, neutralizes, or reduces a biological activity or effect of another molecule, such as a receptor.

The terms “polypeptide”, “oligopeptide”, “peptide” and “protein” are used interchangeably herein to refer to chains of amino acids of any length. The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (-O-) linkage, aryl, alkenyl, cycloalkyl, cycloalkenyl or araldyl. Not all linkages in a polynucleotide need be identical. The preceding description applies to all polynucleotides referred to herein, including RNA and DNA.

In some embodiments, an antibody may be considered to “interact with” an antigen when the equilibrium dissociation constant between the antibody and antigen is, for example, equal to or less than 10000 nM, equal to or less than 2000 nM, equal to or less than 1000 nM, equal to or less than 200 nM, or equal to or less than 20 nM, as measured by the methods disclosed herein in Example 2.

An antibody that “preferentially binds” or “specifically binds” (used interchangeably herein) to an antigen is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a particular epitope is an antibody that binds this epitope with greater affinity, avidity, more readily, and/or with greater duration than it binds to other epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. In addition, an antibody with conditional affinity as provided herein may “specifically bind” to an antigen with different affinities, depending on, for example, the concentration of a small molecule agent.

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected with a polynucleotide(s) of this invention.

As known in the art, the term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant domains, CH2 and CH3. As is known in the art, an Fc region can be present in dimer or monomeric form.

The term “compete”, as used herein with regard to an antibody, means that a first antibody binds to an epitope in a manner sufficiently similar to the binding of a second antibody, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

A “functional Fc region” possesses at least one effector function of a native sequence Fc region. Exemplary “effector functions” include C1q binding; complement dependent cytotoxicity; Fc receptor binding; antibody-dependent cell-mediated cytotoxicity; phagocytosis; down-regulation of cell surface receptors (e.g. B cell receptor), etc. Such effector functions generally require the Fc region to be combined with a binding domain (e.g. an antibody variable domain) and can be assessed using various assays known in the art for evaluating such antibody effector functions.

A “native sequence Fc region” comprises an amino acid sequence identical to the amino acid sequence of an Fc region found in nature. A “variant Fc region” comprises an amino acid sequence which differs from that of a native sequence Fc region by virtue of at least one amino acid modification, yet retains at least one effector function of the native sequence Fc region.

As used herein, “treatment” is an approach for obtaining beneficial or desired clinical results. For purposes of this invention, beneficial or desired clinical results include, but are not limited to, one or more of the following: reducing the proliferation of (or destroying) neoplastic or cancerous cells, inhibiting metastasis of neoplastic cells, shrinking or decreasing the size of a tumor, remission of cancer, decreasing symptoms resulting from cancer, increasing the quality of life of those suffering from cancer, decreasing the dose of other medications required to treat cancer, delaying the progression of cancer, curing a cancer, and/or prolong survival of patients having cancer. Other beneficial or desired clinical results include, for example, any clinical result that may be mediated by an antibody, such as: treatment of cardiovascular disease (e.g. by inhibiting glycoprotein ∥b/|lla or PCSK9); treatment of autoimmune disease (e.g. by inhibiting TNF-alpha or CD11 a); or macular degeneration (e.g. by inhibiting VEGF-A).

“Ameliorating” means a lessening or improvement of one or more symptoms (for example, as compared to not providing an antibody with conditional affinity as provided herein, or to providing a control antibody). “Ameliorating” also includes shortening or reduction in duration of a symptom.

As used herein, an “effective dosage” or “effective amount” of drug, compound, or pharmaceutical composition is an amount sufficient to effect any one or more beneficial or desired results. In more specific aspects, an effective amount prevents, alleviates or ameliorates symptoms of disease, and/or prolongs the survival of the subject being treated. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing one or more symptoms of a disease such as, for example, cancer including, for example without limitation, gastric cancer, sarcoma, lymphoma, Hodgkin’s lymphoma, leukemia, head and neck cancer, squamous cell head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, multiple myeloma, renal cell carcinoma, bladder cancer, cervical cancer, choriocarcinoma, colon cancer, oral cancer, skin cancer, and melanoma, decreasing the dose of other medications required to treat the disease, enhancing the effect of another medication, and/or delaying the progression of the cancer in patients. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

An “individual”, “patient”, or “subject” is any single organism for which therapy is desired or that is participating in a clinical trial, epidemiological study, or used as a control, including for example, humans and other mammals such as monkeys, apes, cows, horses, dogs, cats, mice, and rats.

As used herein, “vector” means a construct, which is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

As used herein, “pharmaceutically acceptable carrier” or “pharmaceutical acceptable excipient” includes any material which, when combined with an active ingredient, allows the ingredient to retain biological activity and is non-reactive with the subject’s immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline (PBS) or normal (0.9%) saline. Compositions comprising such carriers are formulated by well-known conventional methods (see, for example, Remington’s Pharmaceutical Sciences, 18th edition, A. Gennaro, ed., Mack Publishing Co., Easton, PA, 1990; and Remington, The Science and Practice of Pharmacy 20th Ed. Mack Publishing, 2000).

The term “kₒₙ” or “kₐ”, as used herein, refers to the rate constant for association of an antibody to an antigen.

The term “K_{off}” or “K_{d}”, as used herein, refers to the rate constant for dissociation of an antibody from the antibody/antigen complex.

The term “K_{D}”, as used herein, refers to the equilibrium dissociation constant of an antibody-antigen interaction.

Determinations of the association and dissociation rate constants, kₒₙ and k_{off} respectively, to determine K_{D} and other ratios, may be made, for example, using a surface plasmon resonance-based biosensor to characterize an analyte/ligand interaction under conditions where the analyte is monovalent with respect to binding a ligand that is immobilized at low capacity onto a sensor surface via a capture reagent. The analysis may be performed, for example, using a kinetic titration methodology as described in Karlsson et al., Anal. Biochem 349, 136-147, 2006, or using a multi-cycle kinetics analysis. The sensor chip, capturing reagent, and assay buffer employed for a given assay are chosen to give stable capture of ligand onto the sensor surface, minimize non-specific binding of the analyte to the surfaces, and yield analyte-binding responses that are appropriate for kinetic analysis, per the recommendations in Myszka, J. Mol. Recognit 12, 279-284, 1999. The analyte-binding responses per analyte/ligand interaction are double referenced and fit to a 1:1 Langmuir “mass transport limited model” with kₐ, k_{d} and Rₘₐₓ as global parameters as described in Myszka & Morton et al., Biophys. Chem 64, 127-137 (1997). The equilibrium dissociation constant, K_{D}, is deduced from the ratio of the kinetic rate constants, K_{D} = K_{off}/Kₒₙ. Such determinations preferably take place at 25° C. or 37° C.

Reference to “about” a value or parameter herein includes (and describes) embodiments that are directed to that value or parameter per se, as well as to values or parameters that may be as much as 10% below or above the stated numerical value for that parameter. For example, as dose of “about 5 mg/kg” includes 5 mg/kg and also any value between 4.5 mg/kg and 5.5 mg/kg. Where the term “about” is used within the context of a time period (years, months, weeks, days etc.), the term “about” means that period of time plus or minus one amount of the next subordinate time period (e.g. about 1 year means 11-13 months; about 6 months means 6 months plus or minus 1 week; about 1 week means 6-8 days; etc.), or within 10 per cent of the indicated value, whichever is greater.

The term “immune response” refers to any detectable response to a particular substance (such as an antigen or immunogen) by the immune system of a host mammal, such as innate immune responses (e.g., activation of Toll receptor signaling cascade), cell-mediated immune responses (e.g., responses mediated by T cells, such as antigen-specific T cells, and non-specific cells of the immune system), and humoral immune responses (e.g., responses mediated by B cells, such as generation and secretion of antibodies into the plasma, lymph, and/or tissue fluids).

The term “immunogenic” refers to the ability of a substance to cause, elicit, stimulate, or induce an immune response, or to improve, enhance, increase or prolong a pre-existing immune response, against a particular antigen, whether alone or when linked to a carrier, in the presence or absence of an adjuvant.

The term “intradermal administration,” or “administered intradermally,” in the context of administering a substance to a mammal including a human, refers to the delivery of the substance into the dermis layer of the skin of the mammal. The skin of a mammal is composed of an epidermis layer, a dermis layer, and a subcutaneous layer. The epidermis is the outer layer of the skin. The dermis, which is the middle layer of the skin, contains nerve endings, sweat glands and oil (sebaceous) glands, hair follicles, and blood vessels. The subcutaneous layer is made up of fat and connective tissue that houses larger blood vessels and nerves. In contrast in intradermal administration, “subcutaneous administration” refers to the administration of a substance into the subcutaneous layer and “topical administration” refers to the administration of a substance onto the surface of the skin.

The term “neoplastic disorder” refers to a condition in which cells proliferate at an abnormally high and uncontrolled rate, the rate exceeding and uncoordinated with that of the surrounding normal tissues. It usually results in a solid lesion or lump known as “tumor.” This term encompasses benign and malignant neoplastic disorders. The term “malignant neoplastic disorder”, which is used interchangeably with the term “cancer” in the present disclosure, refers to a neoplastic disorder characterized by the ability of the tumor cells to spread to other locations in the body (known as “metastasis”). The term “benign neoplastic disorder” refers to a neoplastic disorder in which the tumor cells lack the ability to metastasize.

The term “preventing” or “prevent” refers to (a) keeping a disorder from occurring or (b) delaying the onset of a disorder or onset of symptoms of a disorder.

The term “tumor-associated antigen” or “TAA” refers to an antigen which is specifically expressed by tumor cells or expressed at a higher frequency or density by tumor cells than by non-tumor cells of the same tissue type. Tumor-associated antigens may be antigens not normally expressed by the host; they may be mutated, truncated, misfolded, or otherwise abnormal manifestations of molecules normally expressed by the host; they may be identical to molecules normally expressed but expressed at abnormally high levels; or they may be expressed in a context or milieu that is abnormal. Tumor-associated antigens may be, for example, proteins or protein fragments, complex carbohydrates, gangliosides, haptens, nucleic acids, or any combination of these or other biological molecules.

It is understood that wherever embodiments are described herein with the language “comprising,” otherwise analogous embodiments described in terms of “consisting of” and/or “consisting essentially of” are also provided.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting. The term “or” when used in the context of a listing of multiple options (e.g. “A, B, or C”) shall be interpreted to include any one or more of the options, unless the context clearly dictates otherwise.

Exemplary methods and materials are described herein, although methods and materials similar or equivalent to those described herein can also be used in the practice or testing of the present invention. The materials, methods, and examples are illustrative only and not intended to be limiting.

### Antibodies with Conditional Affinity

Provided herein are antibodies which have conditional affinity for binding to an antigen. The affinity of an antibody provided herein for an antigen may be affected by the concentration condition of a small molecule agent. Without being bound by theory, a small molecule agent may interact with an antibody provided herein and affect the conformation of one or more the CDRs of the antibody, such that the affinity of the antibody for an antigen is affected by the small molecule agent. In some embodiments, a small molecule agent may alter the structure of an antibody such that, upon the antibody’s interaction with the small molecule agent, the affinity of the antibody for an antigen increases. In other embodiments, a small molecule agent may alter the structure of an antibody such that, upon the antibody’s interaction with the small molecule agent, the affinity of the antibody for an antigen decreases.

In some embodiments, provided herein is an antibody which has a greater affinity for an antigen when the antibody is in a high agent concentration condition as compared to when the antibody is in a low agent concentration condition. Such antibodies have a lower affinity for the antigen when the antibody is a low agent concentration condition. This type of antibody may be characterized as effectively having an “on switch”, which can be turned on by the small molecule agent. In other words, the small molecule agent increases the affinity of the antibody for the antigen.

In other embodiments, provided herein is an antibody which has a lower affinity for an antigen when the antibody is in a high agent concentration condition as compared to when the antibody is in a low agent concentration condition. Such antibodies have a greater affinity for the antigen when the antibody is a low agent concentration condition. This type of antibody may be characterized as effectively having an “off switch”, which can be turned off by the small molecule agent. In other words, the small molecule agent decreases the affinity of the antibody for the antigen.

An antibody with conditional affinity as provided herein may have any general characteristic as provided elsewhere herein for an antibody. For example, an antibody with conditional affinity may contain a heavy chain variable region (“VH”) and a light chain variable region (“VL”).

A small molecule agent may interact with an antibody with conditional affinity provided herein in a variety of ways. For example, a small molecule agent may specifically bind to an antibody.

In some embodiments, a small molecule agent may specifically bind to an antibody at a location in the antibody that is separate from the antigen-binding location of the antibody. In such circumstances, for example, the antigen-binding location of the antibody may be formed by a structure generated by the combination of the VH region and the VL region (e.g. the antigen may bind at an interface between the VH and VL regions), whereas the small molecule agent may specifically bind to the antibody at a location that is entirely within the VH or VL region and which is separate from the antigen-binding portion of the VH or VL region, respectively (e.g. there may be a “pocket” in the VH region to which the small molecule agent can bind and which is separate from the antigen-binding portion of the VH region; alternatively, this type of “pocket” may occur in the VL region). Typically, in embodiments provided herein in which the small molecule agent binds to a location in the antibody with conditional affinity that does not overlap with the antigen-binding location of the antibody, the interaction of the small molecule agent with the antibody with conditional affinity alters the conformation of at least one of the CDRs in the antibody, and thereby alters the affinity of the antibody for an antigen.

In some embodiments, a small molecule agent may specifically bind to an antibody at a location in the antibody that is the same as or that at least partially overlaps with the antigen-binding of location of the antibody. In this situation, at least some of the amino acids of the antibody involved in binding to the small molecule agent are involved in binding to the antigen. In these circumstances, binding of the small molecule agent to the antibody may affect the affinity of the antibody with conditional affinity for the antigen in a variety of ways. For example, the binding of the small molecule agent to the antibody with conditional affinity may alter the conformation of at least one of the CDRs in the antibody, and thereby alter the affinity of the antibody for an antigen. In another example, the binding of the small molecule agent to the antibody with conditional affinity might not alter the conformation of CDRs in the antibody, but instead, sterically or electrostatically affects the binding of the antigen to the antibody (e.g. the small molecule agent may sterically hinder the binding of the antigen to the antibody), and thereby affects the affinity of the antibody for the antigen. In another example, the binding of a small molecule agent to an antibody with conditional affinity may both i) alter the conformation of one or more CDRs in the antibody and ii) sterically or electrostatically affect the binding of an antigen to the antibody, wherein the affinity of the antibody for the antigen is affected by both of these types of changes caused by the binding of the small molecule agent to the antibody with conditional affinity. A small molecule agent may bind to a location in an antibody that overlaps with the antigen-binding location in the antibody, for example, when the small molecule agent is in a greater concentration than the antigen, or when the small molecule agent binds to the antibody with a higher affinity than with which the antigen binds to the antibody.

In some embodiments, a small molecule agent may transiently interact with an antibody with conditional affinity, where such transient interactions affect the conformation of the antibody.

In some embodiments, there may be a “motif” in an antibody with conditional affinity to which a small molecule agent may bind. For example, the motif may be in any of the above-described locations in an antibody in which a small molecule agent may bind. In some embodiments, the agent-binding motif may be in the VH or VL region of an antibody comprising a VH and VL region. The motif may involve consecutive amino acids in the primary structure of a polypeptide of the antibody (e.g. consecutive amino acids within the VH), or the motif may involve amino acids that aren’t in sequential order in the primary structure of the antibody, but which are near each other within the antibody due to the secondary or tertiary structure of the antibody. In some embodiments, an agent binding motif within an antibody with conditional affinity may be within the VH region of the antibody. In some embodiments, an agent binding motif may include the CDR1, CDR2, or CDR3 of the VH region of the antibody, or any combination thereof (e.g. just CDR2, the combination of CDR1 and CDR2, or the combination of CDR1, CDR2, and CDR3). In some embodiments, an agent binding motif may involve a portion of a CDR of a VH region (e.g. CDR1, CDR2, and a portion of the CDR3 of the VH region may be involved in binding to the agent). In some embodiments, the agent-binding motif includes the VH CDR1 and VH CDR2 of an antibody. In some embodiments, the agent-binding motif may include a combination of one or more CDRs and one or more framework regions (FRs).

In some embodiments, the agent-binding motif may be in the VH region of an antibody with conditional affinity that comprises a VH and VL region. In some embodiments, an agent-binding motif in the VH region may comprise the amino acid sequence:

QVQLVESGGGLVQAGGSLRLSCAASRRSSRSWAMHWVRQAPGKGLEWVAV
ISYDGRLKYYADSVKGRFTISRDNAEYLVYLQMNSLRAEDTAVYYCAA

(SEQ ID NO: 1). Insome embodiments, an agent-binding motif in the VH region may comprise the amino acid sequence of a CDR1 and CDR2 of SEQ ID NO. 1, wherein the CDR1 and CDR2 are defined according to any CDR definition provided herein (e.g. Kabat, Chothia, etc.). In some embodiments, an agent-binding motif in the VH region may comprise a CDR1 comprising the amino acid sequence shown in SEQ ID NO: 43 (RRSSRSWAMH), SEQ ID NO: 116 (RRSSRSW), or SEQ ID NO: 117 (SWAMH). In some embodiments, an agent-binding motif in the VH region may comprise a CDR2 comprising the amino acid sequence shown in SEQ ID NO: 44 (VISYDGRLKYYADSVKGRF) or SEQ ID NO: 118 (SYDGRL). In some embodiments, an agent-binding motif in the VH region may comprise a FR3 comprising the amino acid sequence CAA. In some embodiments, an agent-binding motif in the VH region may comprise a FR3 comprising the amino acid sequence YLVY (SEQ ID NO: 165). In some embodiments, an agent-binding motif in the VH region may comprise a CDR1 comprising the amino acid sequence shown in SEQ ID NO: 43 (RRSSRSWAMH), SEQ ID NO: 116 (RRSSRSW), or SEQ ID NO: 117 (SWAMH) and a CDR2 comprising the amino acid sequence shown in SEQ ID NO: 44 (VISYDGRLKYYADSVKGRF) or SEQ ID NO: 118 (SYDGRL). In some embodiments, an agent-binding motif in the VH region may comprise a CDR1 comprising the amino acid sequence shown in SEQ ID NO: 43 (RRSSRSWAMH), SEQ ID NO: 116 (RRSSRSW), or SEQ ID NO: 117 (SWAMH), a CDR2 comprising the amino acid sequence shown in SEQ ID NO: 44 (VISYDGRLKYYADSVKGRF) or SEQ ID NO: 118 (SYDGRL), and a FR3 region comprising the amino acid sequence CAA. In some embodiments, an agent-binding motif in the VH region may comprise a CDR1 comprising the amino acid sequence shown in SEQ ID NO: 43 (RRSSRSWAMH), SEQ ID NO: 116 (RRSSRSW), or SEQ ID NO: 117 (SWAMH), a CDR2 comprising the amino acid sequence shown in SEQ ID NO: 44 (VISYDGRLKYYADSVKGRF) or SEQ ID NO: 118 (SYDGRL), and a FR3 region comprising the amino acid sequences CAA and YLVY (SEQ ID NO: 165). In some embodiments, an agent-binding motif in the VH region may comprise an amino acid sequence that is at least 99%, 98%, 97%, 96%, 95%, 94%, 93%, 92%, 91%, 90%, 85%, 80%, 75%, 70%, 65%, 60%, 55%, or 50% identical to the amino acid sequence of SEQ ID NO. 1. In some embodiments, an agent-binding motif in the VH region may comprise an amino acid sequence comprising at least 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 contiguous amino acids of the sequence of SEQ ID NO. 1. In some embodiments, an agent-binding motif in the VH region may comprise an amino acid sequence comprising at least 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 contiguous amino acids of the sequence of SEQ ID NO. 1, or a variant sequence thereof, wherein the variant sequence contains 1 or more conservative amino acid substitutions in the contiguous amino acid sequence as compared to the original sequence in SEQ ID NO. 1, and wherein no more than 25% of the amino acids in the contiguous sequence are substituted as compared to the original sequence. Thus, for example, for an amino acid sequence comprising 20 contiguous amino acids from SEQ ID NO. 1 or a variant thereof, the variant contiguous sequence may contain up to 5 conservative amino acid substitutions among the 20 contiguous amino acids (25% of 20 is 5).

In some embodiments, a VH region of an antibody with conditional affinity provided herein may comprise the amino acid sequence:

QVQLVESGGGLVQAGGSLRLSCAASRRSSRSWAMHWVRQAPGKGLEWVAV
ISYDGRLKYYADSVKGRFTISRDNAEYLVYLQMNSLRAEDTAVYYCAA

(SEQ ID NO: 1). Insome embodiments, the VH region of an antibody with conditional affinity provided herein may comprise the amino acid sequence of a CDR1 and CDR2 of SEQ ID NO. 1, wherein the CDR1 and CDR2 are defined according to any CDR definition provided herein (e.g. Kabat, Chothia, etc.). In some embodiments, the VH region of an antibody with conditional affinity provided herein may comprise a CDR1 comprising the amino acid sequence shown in SEQ ID NO: 43 (RRSSRSWAMH), SEQ ID NO: 116 (RRSSRSW), or SEQ ID NO: 117 (SWAMH). In some embodiments, the VH region of an antibody with conditional affinity provided herein may comprise a CDR2 comprising the amino acid sequence shown in SEQ ID NO: 44 (VISYDGRLKYYADSVKGRF) or SEQ ID NO: 118 (SYDGRL). In some embodiments, the VH region of an antibody with conditional affinity provided herein may comprise a FR3 comprising the amino acid sequence CAA. In some embodiments, the VH region of an antibody with conditional affinity provided herein may comprise a FR3 comprising the amino acid sequence YLVY (SEQ ID NO: 165). In some embodiments, the VH region of an antibody with conditional affinity provided herein may comprise a CDR1 comprising the amino acid sequence shown in SEQ ID NO: 43 (RRSSRSWAMH), SEQ ID NO: 116 (RRSSRSW), or SEQ ID NO: 117 (SWAMH) and a CDR2 comprising the amino acid sequence shown in SEQ ID NO: 44 (VISYDGRLKYYADSVKGRF) or SEQ ID NO: 118 (SYDGRL). In some embodiments, the VH region of an antibody with conditional affinity provided herein may comprise a CDR1 comprising the amino acid sequence shown in SEQ ID NO: 43 (RRSSRSWAMH), SEQ ID NO: 116 (RRSSRSW), or SEQ ID NO: 117 (SWAMH), a CDR2 comprising the amino acid sequence shown in SEQ ID NO: 44 (VISYDGRLKYYADSVKGRF) or SEQ ID NO: 118 (SYDGRL), and a FR3 region comprising the amino acid sequence CAA. In some embodiments, the VH region of an antibody with conditional affinity provided herein may comprise a CDR1 comprising the amino acid sequence shown in SEQ ID NO: 43 (RRSSRSWAMH), SEQ ID NO: 116 (RRSSRSW), or SEQ ID NO: 117 (SWAMH), a CDR2 comprising the amino acid sequence shown in SEQ ID NO: 44 (VISYDGRLKYYADSVKGRF) or SEQ ID NO: 118 (SYDGRL), and a FR3 region comprising the amino acid sequences CAA and YLVY (SEQ ID NO: 165). In some embodiments, the VH region of an antibody with conditional affinity provided herein may comprise an amino acid sequence that is at least 99%, 98%, 97%, 96%, 95%, 94%, 93%, 92%, 91%, 90%, 85%, 80%, 75%, 70%, 65%, 60%, 55%, or 50% identical to the amino acid sequence of SEQ ID NO. 1. In some embodiments, the VH region of an antibody with conditional affinity provided herein may comprise an amino acid sequence comprising at least 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 contiguous amino acids of the sequence of SEQ ID NO. 1. In some embodiments, the VH region of an antibody with conditional affinity provided herein may comprise an amino acid sequence comprising at least 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 contiguous amino acids of the sequence of SEQ ID NO. 1, or a variant sequence thereof, wherein the variant sequence contains 1 or more conservative amino acid substitutions in the contiguous amino acid sequence as compared to the original sequence in SEQ ID NO: 1, and wherein no more than 25% of the amino acids in the contiguous sequence are substituted as compared to the original sequence.

Optionally, the amino acid sequence of SEQ ID NO: 1 may be referred to as a “partial VH sequence” or the like, as in some embodiments, SEQ ID NO: 1 is a portion of a VH region, wherein SEQ ID NO: 1 comprises a VH CDR1 and VH CDR2 sequence.

In some embodiments, binding of a small molecule agent at the agent-binding motif of the antibody may result in a conformational change in one or more CDRs of the antibody. The conformational change of the one or more CDRs resulting from the binding of the small molecule may result in a change in the affinity of the antibody for an antigen. Without being bound by theory, small molecule agents may alter the affinity of antibodies provided herein for antigens according to this mechanism.

In some embodiments, binding of a small molecule agent to an antibody provided herein may decrease the affinity of the antibody for an antigen by serving as a competitor which competes with the antigen for binding to the antibody. In such embodiments, optionally, the small molecule agent doesn’t necessarily alter the conformation of one or more CDRs of the antibody, but still may reduce the affinity of the antibody for the antigen by interfering with the antibody-antigen interaction. In some embodiments, a small molecule agent may decrease the affinity of an antibody with conditional affinity for an antigen by one or both of: i) physically interfering with the antibody-antigen interaction and ii) altering the conformation of one or more CDRs of the antibody.

In some embodiments, binding of a small molecule agent to an antibody provided herein may increase the affinity of the antibody for an antigen by sterically or electrostatically aiding the binding of the antibody to the antigen (e.g. the antibody may have more contacts with the antigen when the antibody is also bound to the small molecule agent, as compared to when the antibody is not bound to the small molecule agent). In such embodiments, optionally, the small molecule agent doesn’t necessarily alter the conformation of one or more CDRs of the antibody, but still may increase the affinity of the antibody for the antigen by providing, for example, a charge or structure that facilitates the antibody-antigen interaction. In some embodiments, a small molecule agent may increase the affinity of an antibody with conditional affinity for an antigen by one or both of: i) sterically or electrostatically promoting the antibody-antigen interaction, and ii) altering the conformation of one or more CDRs of the antibody.

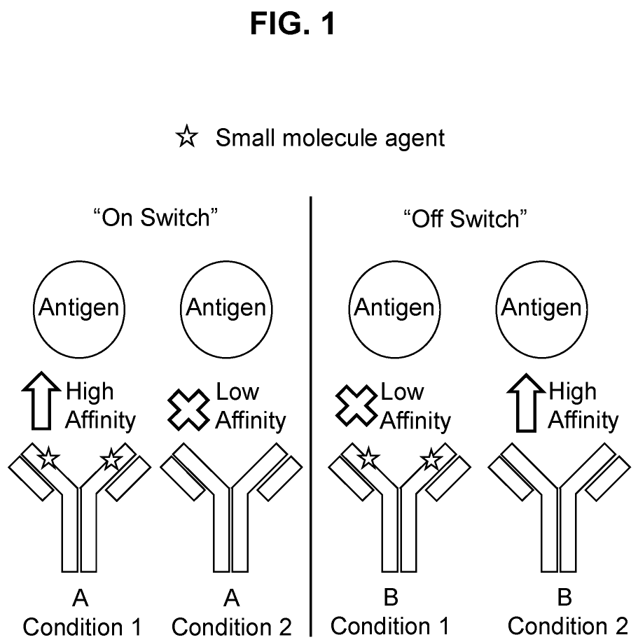

FIG. 1 provides schematics of exemplary antibodies with conditional affinity and conditions provided herein. In FIG. 1, the star shape represents a small molecule agent, the Y shape represents an antibody (having 2 heavy chains and 2 light chains), and the round shape represents an antigen. The left-side panel depicts an antibody which has a greater affinity for an antigen when the antibody is in a high agent concentration condition as compared to when the antibody is in a low agent concentration condition (antibody “A”). In particular, the left-side panel depicts this type of antibody 1) when bound to a small molecule agent / in a high agent concentration condition (“A Condition 1”) and ii) when not bound to the small molecule agent / in a low agent concentration condition (“A Condition 2”). As depicted in FIG. 1, when antibody A is bound to a small molecule agent / is in a high agent concentration condition, it has a high affinity for the antigen. As also depicted in FIG. 1, when antibody A is not bound to the small molecule agent / is in a low agent concentration condition, it has low affinity for the antigen. The right-side panel of FIG. 1 depicts an antibody which has a greater affinity for an antigen when the antibody is in a low agent concentration condition as compared to when the antibody is in a high agent concentration condition (antibody “B”). In particular, the right-side panel depicts this type of antibody 1) when bound to a small molecule agent / in a high agent concentration condition (“B Condition 1”) and ii) when not bound to the small molecule agent / in a low agent concentration condition (“B Condition 2”). As depicted in FIG. 1, when antibody B is bound to a small molecule agent / is in a high agent concentration condition, it has a low affinity for the antigen. As also depicted in FIG. 1, when antibody B is not bound to the small molecule agent / is in a low agent concentration condition, it has high affinity for the antigen. As additionally depicted in FIG. 1, in some embodiments, a small molecule agent may bind to an antibody with conditional affinity provided herein at a location in the heavy chain of the antibody.

In some embodiments, an antibody having conditional affinity provided herein may be a single domain antibody (e.g. a VHH fragment), wherein the single domain has affinity for both an antigen and a small molecule agent, and wherein the small molecule agent affects the affinity of the antibody for the antigen. Optionally, the single domain antibody may contain any of the sequences or have any of the characteristics of a VH region provided herein.

Small molecule agents as provided herein may be of various different chemical classes. Typically, small molecule agents are organic molecules which contain functional groups which facilitate the interaction of the small molecule agent with proteins (e.g. through hydrogen bonding). Small molecule agents may contain, for example, one or more amine, carbonyl, hydroxyl, sulfhydryl or carboxyl groups. In some embodiments, a small molecule agent may be folic acid, an inhibitor of dihydrofolate reductase (DHFR) (e.g. methotrexate or aminopterin), sulfasalazine, hydroxychloroquine, leflunomide, imatinib, gefitinib, erlotinib, sorafenib, abiraterone, crizotinib, vemurafenib, vismodegib, sonidegib, everolimus, tamoxifen, toremifene, fulvestrant, anastrozole, exemestane, lapatinib, letrozole, emtansine, palbociclib, ziv-aflibercept, regorafenib, imatinib mesylate, lanreotide acetate, sunitinib, alitretinoin, pazopanib, temsirolimus, axitinib, tertinoin, dasatinib, nilotinib, bosutinib, ibrutinib, idelalisib, gefitinib, afatinib dimaleate, ceritinib, denileukin diftitox, vorinostat, romidepsin, bexarotene, bortezomib, pralatrexate, lometrexol, AG2034, GW1843, piritrexim, talotrexin, nolatrexed, plevitrexed, BGC945, lenalimodie, belinostat, vemurafenib, trametinib, dabrafenib, carfilzomib, lenaliomide, pomalidomide, panobinostat, ruxolitinib phosphate, cabozantinib, lenvatinib mesylate, or a salt (e.g. pharmaceutically acceptable salts), amide, ester, or variant thereof. Small molecule agents may be provided to a subject, for example, by a topical, enteral, or parenteral (e.g. intravenous, intramuscular, or intrathecal) route.

In some embodiments, a small molecule agent is an inhibitor of dihydrofolate reductase (DHFR). DHFR is an enzyme which reduces dihydrofolic acid to tetrahydrofolic acid. Tetrahydrofolic acid is required in cells for the synthesis of various molecules required for cell growth, such as purines and thymidylic acid, and thus, inhibition of DHFR results in impairment of cell growth and proliferation. Accordingly, inhibitors of DHFR are useful as anti-cell proliferation and anti-cancer agents. Certain DHFR inhibitors selectively inhibit specific bacterial DHFR proteins (rather than mammalian DHFR) and thus are useful as anti-bacterial agents. In some embodiments, a DHFR inhibitor is a bacterial DHFR inhibitor. Exemplary bacterial DHFR inhibitors include aditoprim, brodimoprim, iclaprim, tetroxoprim, and trimethoprim.

In some embodiments, a DHFR inhibitor is a mammalian DHFR inhibitor. Exemplary mammalian DHFR inhibitors include aminopterin, methotrexate, pemetrexed, pralatrexate, raltitrexed, and trimetrexate.

In some embodiments, a small molecule agent is methotrexate. Methotrexate has the IUPAC / systematic name (2S)-2-[(4-{[(2,4-Diaminopteridin-6-yl)methyl](methyl)amino}benzoyl)amino]pentanedioic acid, the chemical formula C₂₀H₂₂N₈O₅, and may be identified by the PubChem CID # 126941. Methotrexate is a competitive inhibitor of DHFR, and binds DHFR with high affinity. In some embodiments, methotrexate may be formulated as a pharmaceutically acceptable salt of methotrexate (e.g. a disodium salt).

In some embodiments, a small molecule agent may be non-naturally occurring in a subject (i.e. it is not synthesized by a subject, and is not a normal component of a diet of the subject). For example, MTX does not occur naturally in humans.

In some embodiments, a small molecule agent provided herein may be chemically modified such that it is linked to another molecule (the other molecule may be optionally referred to herein as a “conjugate molecule”), resulting in improved properties of the small molecule agent (such as greater solubility, improved stability, etc.). In embodiments in which a small molecule agent provided herein is linked to another molecule, the conjugate molecule does not specifically bind to the corresponding antibody with conditional affinity provided herein (i.e. the binding of the small molecule agent-conjugate molecule combination to the antibody with conditional affinity is mediated by the small molecule agent, rather than the conjugate molecule). In some embodiments, the linking of a small molecule agent to a conjugate molecule may further improve the effectiveness of the small molecule agent at affecting the affinity of a corresponding antibody with conditional affinity for an antigen. For example, in some embodiments, if the binding of a small molecule agent to an antibody with conditional affinity reduces the affinity of the antibody for an antigen, if that small molecule agent is chemically linked to a conjugate molecule (e.g. PEG), then the chemically-modified version of the small molecule agent may be more effective at reducing affinity of the antibody for the antigen than the unmodified version of the small molecule agent. In another example, in some embodiments, if the binding of a small molecule agent to an antibody with conditional affinity increases the affinity of the antibody for an antigen, if that small molecule agent is chemically linked to a conjugate molecule (e.g. PEG), then the chemically-modified version of the small molecule agent may be more effective at increasing affinity of the antibody for the antigen than the unmodified version of the small molecule agent. Exemplary conjugate molecules that may be used with small molecule agents include, for example, polyethylene glycol (PEG), polyglutamate, biotin, PAS (Pro, Ala, and Ser) amino acid sequences, short peptides (e.g. peptides containing 10 amino acids or fewer, 5 amino acids or fewer, or 3 amino acids or fewer), or Fc fragments. References herein to a particular small molecule agent (e.g. MTX) include that small molecule agent linked to a conjugate molecule, unless context clearly dictates otherwise (e.g. a reference herein to “MTX” includes, for example, unmodified MTX as well as MTX linked to a conjugate molecule).

Without being bound by theory, a small molecule agent that reduces the affinity of an antibody with conditional affinity for an antigen may, in some embodiments, be more effective at reducing the affinity of the antibody for the antigen when the small molecule agent is linked to a conjugate molecule than when the small molecule agent is un-modified, according to the following mechanism: The small molecule agent may reduce the affinity of the antibody with conditional affinity for an antigen by binding to the antibody, and, in doing so, it may affect the position of one or more of the CDRs in the antibody; the antibody with the re-positioned CDRs may have reduced affinity for the antigen. If that same small molecule agent is linked to a conjugate molecule, the small molecule agent may still bind to the antibody with conditional affinity and cause the same effect on the position of the CDRs in the antibody, and, in addition, the conjugate molecule may provide further impediments to the antibody-antigen interaction (e.g. when the small molecule agent-conjugate molecule is bound to the antibody with conditional affinity, the conjugate molecule may sterically hinder or provide an electrostatic charge which impedes antibody-antigen interaction).

Without being bound by theory, a small molecule agent that increases the affinity of an antibody with conditional affinity for an antigen may, in some embodiments, be more effective at increasing the affinity of the antibody for the antigen when the small molecule agent is linked to a conjugate molecule than when the small molecule agent is un-modified, according to the following mechanism: The small molecule agent may increase the affinity of the antibody with conditional affinity for an antigen by binding to the antibody, and, in doing so, it may affect the position of one or more of the CDRs in the antibody; the antibody with the re-positioned CDRs may have increased affinity for the antigen. If that same small molecule agent is linked to a conjugate molecule, the small molecule agent may still bind to the antibody with conditional affinity and cause the same effect on the position of the CDRs in the antibody, and, in addition, the conjugate molecule may provide one or more effects that further promote to the antibody-antigen interaction (e.g. when the small molecule agent-conjugate molecule is bound to the antibody with conditional affinity, the conjugate molecule may sterically promote or provide an electrostatic charge which increases antibody-antigen interaction).

In some embodiments, for an antibody and its antigen as provided herein, the ratio of: A) the antibody-antigen K_{D} at a high agent concentration condition over B) the antibody-antigen K_{D} at a low agent concentration condition (i.e. antibody-antigen K_{D} at a high agent concentration condition / antibody-antigen K_{D} at a low agent concentration condition) at 25° C. is at least 1.5, 2, 3, 4, 5, 6, 7, 8, 9, 10, 15, 16, 20, 25, 30, 40, 50, 100, 200, 300, 400, 500, or 1000, or ranges between any of 1.5, 2, 3, 4, 5, 6, 7, 8, 9, 10, 15, 20, 25, 30, 40, 50, 100, 200, 300, 400, or 500 and any of 2, 3, 4, 5, 6, 7, 8, 9, 10, 15, 20, 25, 30, 40, 50, 100, 200, 300, 400, 500, or 1000, in which the second value is larger than the first value. In such circumstances, the K_{D} at high agent concentration conditions is a larger number than the K_{D} at low agent concentration conditions. Since a higher K_{D} number indicates a lower antibody-antigen affinity, in these circumstances, the antibody has a lower affinity for its antigen when in high agent concentration conditions as compared to when in low agent concentration conditions. Thus, in these circumstances, the agent effectively reduces the affinity of the antibody for its antigen (i.e. the agent effectively activates an “off switch” in the antibody).

Similarly, for an antibody and its antigen as provided herein, in some embodiments, the ratio of: A) the antibody-antigen k_{off} at a high agent concentration condition over B) the antibody-antigen k_{off} at a low agent concentration condition (i.e. antibody-antigen k_{off} at a high agent concentration condition / antibody-antigen k_{off} at a low agent concentration condition) at 25° C. is at least 1.5, 2, 3, 4, 5, 6, 7, 8, 9, 10, 15, 16, 20, 25, 30, 40, 50, 100, 200, 300, 400, 500, or 1000, or ranges between any of 1.5, 2, 3, 4, 5, 6, 7, 8, 9, 10, 15, 20, 25, 30, 40, 50, 100, 200, 300, 400, or 500 and any of 2, 3, 4, 5, 6, 7, 8, 9, 10, 15, 20, 25, 30, 40, 50, 100, 200, 300, 400, 500, or 1000, in which the second value is larger than the first value.

In embodiments provided herein in which an antibody has a lower affinity for its antigen when in high agent concentration conditions as compared to when in low agent concentration conditions, the K_{D} of the antibody-antigen interaction at 25° C. under low agent conditions may be, for example, less than any of 500 µM, 200 µM, 100 µM, 50 µM, 20 µM, 10 µM, 5 µM, 2 µM, 1 µM, 500 nM, 200 nM, 100 nM, 50 nM, 20 nM, 10 nM, 5 nM, 2 nM, 1 nM, 500 pM, 200 pM, 100 pM, 50 pM, 20 pM, 10 nM, 5 pM, 2 pM, or 1 pM, between 0.01 nM and 100 nM, between 0.1 nM and 10 nM, or between any of 1 pM, 2 pM, 5 pM, 10 pM, 20 pM, 50 pM, 100 pM, 200 pM, 500 pM, 1 nM, 2 nM, 5 nM, 10 nM, 20 nM, 50 nM, 100 nM, 200 nM, 500 nM, 1 µM, 2 µM, 5 µM, 10 µM, 20 µM, 50 µM, 100 µM, 200 µM, or 500 µM and any of 2 pM, 5 pM, 10 pM, 20 pM, 50 pM, 100 pM, 200 pM, 500 pM, 1 nM, 2 nM, 5 nM, 10 nM, 20 nM, 50 nM, 100 nM, 200 nM, 500 nM, 1 µM, 2 µM, 5 µM, 10 µM, 20 µM, 50 µM, 100 µM, 200 µM, 500 µM or 800 µM, in which the second value is larger than the first value. In embodiments provided herein in which an antibody has a lower affinity for its antigen when in high agent concentration conditions as compared to when in low agent concentration conditions, the k_{off} of the antibody-antigen interaction at 25° C. under low agent concentration conditions may be, for example, less than any of 1×10 E⁻¹ s⁻¹, 1×10 E-² S⁻¹, 1×10 E⁻³ S⁻¹, or 1×10 E⁻⁴ S⁻¹, between 1×10 E⁻⁴ S⁻¹ and 1×10 E⁻¹ s⁻¹, between 1×10 E⁻³ S⁻¹ and 1×10 E⁻¹ s⁻¹, between 1×10 E-² S⁻¹ and 1x10 E⁻¹ s⁻¹, between 1×10 E⁻⁴ S⁻¹ and 1×10 E⁻³ s⁻¹, between 1×10 E⁻⁴ S⁻¹ and 1×10 E⁻² S⁻¹, or between 1×10 E⁻³ S⁻¹ and 1×10 E⁻² S⁻¹.

In some embodiments, for an antibody and its antigen as provided herein, the ratio of: A) the antibody-antigen K_{D} at a low agent concentration condition over B) the antibody-antigen K_{D} at a high agent concentration condition (i.e. antibody-antigen K_{D} at a low agent concentration condition / antibody-antigen K_{D} at a high agent concentration condition) at 25° C. is at least 1.5, 2, 3, 4, 5, 6, 7, 8, 9, 10, 15, 16, 20, 25, 30, 40, 50, 100, 200, 300, 400, 500, or 1000, or ranges between any of 1.5, 2, 3, 4, 5, 6, 7, 8, 9, 10, 15, 20, 25, 30, 40, 50, 100, 200, 300, 400, or 500 and any of 2, 3, 4, 5, 6, 7, 8, 9, 10, 15, 20, 25, 30, 40, 50, 100, 200, 300, 400, 500, or 1000, in which the second value is larger than the first value. In such circumstances, the K_{D} at low agent concentration conditions is a larger number than the K_{D} at high agent concentration conditions. Since a higher K_{D} number indicates a lower antibody-antigen affinity, in these circumstances, the antibody has a lower affinity for its antigen when in low agent concentration conditions as compared to when in high agent concentration conditions. Thus, in these circumstances, the agent effectively increases the affinity of the antibody for its antigen (i.e. the agent effectively activates an “on switch” in the antibody).

Similarly, for an antibody and its antigen as provided herein, in some embodiments, the ratio of: A) the antibody-antigen k_{off} at a low agent concentration condition over B) the antibody-antigen k_{off} at a high agent concentration condition (i.e. antibody-antigen k_{off} at a low agent concentration condition / antibody-antigen k_{off} at a high agent concentration condition) at 25° C. is at least 1.5, 2, 3, 4, 5, 6, 7, 8, 9, 10, 15, 16, 20, 25, 30, 40, 50, 100, 200, 300, 400, 500, or 1000, or ranges between any of 1.5, 2, 3, 4, 5, 6, 7, 8, 9, 10, 15, 20, 25, 30, 40, 50, 100, 200, 300, 400, or 500 and any of 2, 3, 4, 5, 6, 7, 8, 9, 10, 15, 20, 25, 30, 40, 50, 100, 200, 300, 400, 500, or 1000, in which the second value is larger than the first value.

In embodiments provided herein in which an antibody has a lower affinity for its antigen when in low agent concentration conditions as compared to when in high agent concentration conditions, the K_{D} of the antibody-antigen interaction at 25° C. under high agent conditions may be, for example, less than any of 500 µM, 200 µM, 100 µM, 50 µM, 20 µM, 10 µM, 5 µM, 2 µM, 1 µM, 500 nM, 200 nM, 100 nM, 50 nM, 20 nM, 10 nM, 5 nM, 2 nM, 1 nM, 500 pM, 200 pM, 100 pM, 50 pM, 20 pM, 10 nM, 5 pM, 2 pM, or 1 pM, between 0.01 nM and 100 nM, between 0.1 nM and 10 nM, or between any of 1 pM, 2 pM, 5 pM, 10 pM, 20 pM, 50 pM, 100 pM, 200 pM, 500 pM, 1 nM, 2 nM, 5 nM, 10 nM, 20 nM, 50 nM, 100 nM, 200 nM, 500 nM, 1 µM, 2 µM, 5 µM, 10 µM, 20 µM, 50 µM, 100 µM, 200 µM, or 500 µM and any of 2 pM, 5 pM, 10 pM, 20 pM, 50 pM, 100 pM, 200 pM, 500 pM, 1 nM, 2 nM, 5 nM, 10 nM, 20 nM, 50 nM, 100 nM, 200 nM, 500 nM, 1 µM, 2 µM, 5 µM, 10 µM, 20 µM, 50 µM, 100 µM, 200 µM, 500 µM or 800 µM, in which the second value is larger than the first value. In embodiments provided herein in which an antibody has a lower affinity for its antigen when in low agent concentration conditions as compared to when in high agent concentration conditions, the k_{off} of the antibody-antigen interaction at 25° C. under high agent concentration conditions may be, forexample, less than any of 1×10 E⁻¹ s⁻¹, 1×10 E-² S⁻¹, 1×10 E⁻³ S⁻¹, or 1×10 E⁻⁴ s⁻¹, between 1×10 E⁻⁴ S⁻¹ and 1×10 E⁻¹ s⁻¹, between 1×10 E⁻³ S⁻¹ and 1×10 E⁻¹ s⁻¹, between 1×10 E-² S⁻¹ and 1×10 E⁻¹ s⁻¹, between 1×10 E⁻⁴ S⁻¹ and 1×10 E⁻³ s⁻¹, between1×10 E⁻⁴ S⁻¹ and 1×10 E⁻² S⁻¹, or between 1×10 E⁻³ S⁻¹ and 1×10 E⁻² S⁻¹.

In some embodiments, a “low agent concentration” as provided herein is less than any of 1 mM, 500 µM, 200 µM, 100 µM, 50 µM, 20 µM, 10 µM, 5 µM, 2 µM, 1 µM, 500 nM, 200 nM, 100 nM, 50 nM, 20 nM, 10 nM, 5 nM, 2 nM, 1 nM, 500 pM, 200 pM, 100 pM, 50 pM, 20 pM, 10 nM, 5 pM, 2 pM, or 1 pM, or between 0 pM and any of 1 mM, 500 µM, 200 µM, 100 µM, 50 µM, 20 µM, 10 µM, 5 µM, 2 µM, 1 µM, 500 nM, 200 nM, 100 nM, 50 nM, 20 nM, 10 nM, 5 nM, 2 nM, 1 nM, 500 pM, 200 pM, 100 pM, 50 pM, 20 pM, 10 nM, 5 pM, 2 pM, or 1 pM. For example, in some embodiments, a low agent concentration is about 0 nM to about 0.1 nM, or about 0 nM to about 10 nM.

In some embodiments, a “high agent concentration” as provided herein is more than any of 500 mM, 100 mM, 10 mM, 1 mM, 500 µM, 200 µM, 100 µM, 50 µM, 20 µM, 10 µM, 5 µM, 2 µM, 1 µM, 500 nM, 200 nM, 100 nM, 50 nM, 20 nM, 10 nM, 5 nM, 2 nM, 1 nM, 500 pM, 200 pM, 100 pM, 50 pM, 20 pM, 10 nM, 5 pM, 2 pM, or 1 pM, or between any of 1 pM, 2 pM, 5 pM, 10 pM, 20 pM, 50 pM, 100 pM, 200 pM, 500 pM, 1 nM, 2 nM, 5 nM, 10 nM, 20 nM, 50 nM, 100 nM, 200 nM, 500 nM, 1 µM, 2 µM, 5 µM, 10 µM, 20 µM, 50 µM, 100 µM, 200 µM, 500 µM, 1 mM, 10 mM, 100 mM, or 500 mM and any of 2 pM, 5 pM, 10 pM, 20 pM, 50 pM, 100 pM, 200 pM, 500 pM, 1 nM, 2 nM, 5 nM, 10 nM, 20 nM, 50 nM, 100 nM, 200 nM, 500 nM, 1 µM, 2 µM, 5 µM, 10 µM, 20 µM, 50 µM, 100 µM, 200 µM, 500 µM, 800 µM, 1 mM, 10 mM, 100 mM, 500 mM, or 1 M, in which the second value is larger than the first value. For example, in some embodiments, a high agent concentration is about 1 nM to about 500 nM, or about 50 nM to about 500 nM.

In some embodiments, the agent is methotrexate, and the high agent concentration is any of about 5 µM, 10 µM, 15 µM, 20 µM, 25 µM, 50 µM, 100 µM, 500 µM, 1 mM, 2 mM, 5 mM, 10 mM, 20 mM, 50 mM, 100 mM, 200 mM, 500 mM, 1 M or greater, and the low agent concentration is any of about 500 µM, 100 µM, 50 µM, 20 µM, 10 µM, 5 µM, 2 µM, 1 µM, 0.5 µM, 0.1 µM, or less (e.g. the low agent concentration may be 0 µM), in which the high agent concentration is greater than the low agent concentration. In some embodiments, the agent is methotrexate, and A) the high agent concentration condition is between any of 1 µM, 5 µM, 10 µM, 15 µM, 20 µM, 25 µM, 50 µM, 100 µM, 500 µM, 1 mM, 2 mM, 5 mM, 10 mM, 20 mM, or 50 mM and any of 5 µM, 10 µM, 15 µM, 20 µM, 25 µM, 50 µM, 100 µM, 500 µM, 1 mM, 2 mM, 5 mM, 10 mM or 20 mM, 50 mM, 100 mM, 200 mM, 500 mM, or 1 M wherein the second value is larger than the first value, and B) the low agent concentration condition is between any of 10 mM, 1 mM, 500 µM, 100 µM, 50 µM, 20 µM, 5 µM, 2 µM, 1 µM, 0.5 µM, or y, and any of 100 µM, 50 µM, 20 µM, 5 µM, 2 µM, 1 µM, 0.5 µM, 0.1 µM, or 0 µM, wherein the second value is less than the first value, and wherein the entire high agent concentration range is greater than the entire low agent concentration range.

In some embodiments, for a small molecule agent provided herein, the “low agent concentration” and the “high agent concentration” may be any value wherein the ratio of high agent concentration / low agent concentration is at least 2, 3, 4, 5, 6, 7, 8, 9, 10, 15, 20, 25, 30, 40, 50, 100, 200, or 500, or is between any of 1, 2, 3, 4, 5, 6, 7, 8, 9, 10, 15, 20, 25, 30, 40, 50, 100, or 200 and any of 2, 3, 4, 5, 6, 7, 8, 9, 10, 15, 20, 25, 30, 40, 50, 100, 200, or 500, in which the second value is larger than the first value. When the ratio of high agent concentration / low agent concentration is, for example “4”, the relationship between the high agent concentration and low agent concentration may also be described as that the high agent concentration is 4 times (4x) greater than the low agent concentration. Other ratio values may also be described in the same way. Similarly, in some embodiments, when the “low agent concentration” and the “high agent concentration” are provided in ranges, the ratio of maximum high agent concentration value / maximum low agent concentration value is at least 2, 3, 4, 5, 6, 7, 8, 9, 10, 15, 20, 25, 30, 40, 50, 100, 200, or 500, or is between any of 1, 2, 3, 4, 5, 6, 7, 8, 9, 10, 15, 20, 25, 30, 40, 50, 100, or 200 and any of 2, 3, 4, 5, 6, 7, 8, 9, 10, 15, 20, 25, 30, 40, 50, 100, 200, or 500, in which the second value is larger than the first value. In some embodiments, when the “low agent concentration” and the “high agent concentration” are provided in ranges, the ratio of minimum high agent concentration value / minimum low agent concentration value is at least 2, 3, 4, 5, 6, 7, 8, 9, 10, 15, 20, 25, 30, 40, 50, 100, 200, or 500, or is between any of 1, 2, 3, 4, 5, 6, 7, 8, 9, 10, 15, 20, 25, 30, 40, 50, 100, or 200 and any of 2, 3, 4, 5, 6, 7, 8, 9, 10, 15, 20, 25, 30, 40, 50, 100, 200, or 500, in which the second value is larger than the first value. In some embodiments, when the “low agent concentration” and the “high agent concentration” are provided in ranges, the ratio of minimum high agent concentration value / maximum low agent concentration value is at least 2, 3, 4, 5, 6, 7, 8, 9, 10, 15, 20, 25, 30, 40, 50, 100, 200, or 500, or is between any of 1, 2, 3, 4, 5, 6, 7, 8, 9, 10, 15, 20, 25, 30, 40, 50, 100, or 200 and any of 2, 3, 4, 5, 6, 7, 8, 9, 10, 15, 20, 25, 30, 40, 50, 100, 200, or 500, in which the second value is larger than the first value.

In some embodiments, any of the references herein to “low agent concentration conditions” or “high agent concentration conditions” may include any of the low agent concentration or high agent concentration values or ratios between such concentration conditions provided herein.

The concentration of agents provided herein may refer to, for example, the concentration of a small molecule agent in any solution or suspension of interest, such as, for example, in vitro reaction mixtures or bodily fluids such as serum, plasma, whole blood, saliva, or urine. Thus, reference to a “high agent concentration” and “low agent concentration” of a small molecule agent may refer to, for example, different serum concentrations of the small molecule agent.

In some embodiments, an antibody provided herein may specifically bind to a small molecule agent. In some embodiments, the K_{D} of the antibody-small molecule interaction at 25° C. is less than any of 500 µM, 200 µM, 100 µM, 50 µM, 20 µM, 10 µM, 5 µM, 2 µM, 1 µM, 500 nM, 200 nM, 100 nM, 50 nM, 20 nM, 10 nM, 5 nM, 2 nM, 1 nM, 500 pM, 200 pM, 100 pM, 50 pM, 20 pM, 10 nM, 5 pM, 2 pM, or 1 pM, between 0.01 nM and 100 nM, between 0.1 nM and 10 nM, or between any of 1 pM, 2 pM, 5 pM, 10 pM, 20 pM, 50 pM, 100 pM, 200 pM, 500 pM, 1 nM, 2 nM, 5 nM, 10 nM, 20 nM, 50 nM, 100 nM, 200 nM, 500 nM, 1 µM, 2 µM, 5 µM, 10 µM, 20 µM, 50 µM, 100 µM, 200 µM, or 500 µM and any of 2 pM, 5 pM,

10 pM, 20 pM, 50 pM, 100 pM, 200 pM, 500 pM, 1 nM, 2 nM, 5 nM, 10 nM, 20 nM, 50 nM, 100 nM, 200 nM, 500 nM, 1 µM, 2 µM, 5 µM, 10 µM, 20 µM, 50 µM, 100 µM, 200 µM, 500 µM or 800 µM, in which the second value is larger than the first value.

In some embodiments, it is desirable to use a small molecule agent which has a relatively high affinity (i.e. low K_{D} value) for an antibody with conditional affinity provided herein, so that only relatively low concentrations of the small molecule agent are needed to affect the affinity of the antibody with conditional affinity for an antigen. This may be desirable, for example, if the small molecule agent and the antibody with conditional affinity are to be administered to a subject, and the small molecule agent is toxic to the subject.

In some embodiments, an antibody provided herein may have a greater affinity for a small molecule agent than for an antigen that the antibody also specifically binds. In other embodiments, an antibody provided herein may have a lower affinity for a small molecule agent than for an antigen that the antibody also specifically binds. In some embodiments, an antibody provided herein has a greater affinity for a small molecule agent than for an antigen that the antibody also specifically binds when the antibody, antigen, and agent are in high agent concentration conditions, but has a lower affinity for the small molecule agent than for the antigen when the antibody, antigen, and agent are in low agent concentration conditions. In some embodiments, an antibody provided herein has a greater affinity for a small molecule agent than for an antigen that the antibody also specifically binds when the antibody, antigen, and agent are in low agent concentration conditions, but has a lower affinity for the small molecule agent than for the antigen when the antibody, antigen, and agent are in high agent concentration conditions. In some embodiments, the affinity of an antibody provided herein does not substantially change for a small molecule agent depending on the concentration of the small molecule agent - i.e. the affinity of the antibody for the small molecule may be about the same under both high agent concentration conditions and low agent concentration conditions. Thus, in embodiments provided herein wherein the affinity of an antibody for a small molecule agent is compared to the affinity of that antibody for an antigen, in some circumstances, the affinity of the antibody for the antigen may vary with the concentration of the agent, whereas the affinity of the antibody for the small molecule agent does not vary with the concentration of the agent.

An antibody with conditional affinity as provided herein may be exposed to different concentrations of small molecule agent to an antigen in any temporal sequence. For example, in some embodiments, an antibody with conditional affinity may be exposed to an antigen to which the antibody binds before it is exposed to a small molecule agent that affects the affinity of the antibody for the antigen. Thus, for example, in the case of an antibody that has a higher affinity for the antigen in a high concentration condition of small molecule agent, if the antibody is first exposed to the antigen in a low concentration condition of small molecule agent, the antibody will not bind to the antigen (or only will bind with low affinity). Then, if the small molecule agent is introduced into the environment containing the antibody with conditional affinity and the antigen such that the concentration of the small molecule agent increases, the affinity of the antibody for the antigen may then increase. In other embodiments, an antibody with conditional affinity provided herein may be exposed to a high concentration condition of small molecule agent before the antibody is exposed to the antigen.

In some embodiments, an antibody with conditional affinity provided herein may first bind to a small molecule agent, and then after binding to the small molecule agent (and while remaining bound to the small molecule agent), bind to an antigen. In such embodiments, commonly the binding of the small molecule agent to the antibody with conditional affinity increases the affinity of the antibody-antigen interaction. However in some cases of the above embodiment, the binding of the small molecule agent to the conditionally antibody with conditional affinity decreases the affinity of the antibody-antigen interaction.

In some embodiments, an antibody with conditional affinity provided herein may first bind to an antigen, and then after binding to the antigen, bind to a small molecule agent. In such embodiments, commonly the binding of the small molecule agent to the antibody with conditional affinity decreases the affinity of the antibody-antigen interaction. However in some cases of the above embodiment, the binding of the small molecule agent to the antibody with conditional affinity increases the affinity of the antibody-antigen interaction.

In some embodiments, an antibody provided herein may contain the amino acid sequence Cys-Ala-Ala (“CAA”) in the VH region. In some embodiments, the CAA sequence may be the third framework region (“FR3”) of the VH region (according to both the Kabat and Chothia definitions). In some embodiments, the CAA sequence is the last three amino acids of the VH FR3. In some embodiments, the CAA sequence may be important for the antibody to bind to the small molecule agent, and/or for the small molecule agent to affect the affinity of the antibody for its antigen. In some embodiments, an antibody provided here has greater affinity for a small molecule agent if it contains the amino acid sequence CAA in the VH FR3 than if it does not contain the amino acid sequence CAA in the VH FR3. In some embodiments, the combination of VH CDR1, VH CDR2, and the sequence CAA in VH FR3 may be involved in the binding of a small molecule agent to an antibody with conditional affinity provided herein. In some embodiments, the amino acid sequence CAA in VH FR3 is important for the binding of the small molecule agent methotrexate to an antibody with conditional affinity provided herein, such that methotrexate more readily binds to the antibody if the antibody contains the sequence CAA in the VH FR3 than if the antibody does not contain the sequence CAA in the VH FR3.

In some embodiments, an antibody provided herein may contain the amino acid sequence Tyr-Leu-Val-Tyr (“YLVY”; SEQ ID NO: 165) in the VH region. In some embodiments, the YLVY sequence may be the third framework region (“FR3”) of the VH region (according to both the Kabat and Chothia definitions). In some embodiments, the YLVY sequence may be important for the antibody to bind to the small molecule agent, and/or for the small molecule agent to affect the affinity of the antibody for its antigen. In some embodiments, an antibody provided here has greater affinity for a small molecule agent if it contains the amino acid sequence YLVY in the VH FR3 than if it does not contain the amino acid sequence YLVY in the VH FR3. In some embodiments, the combination of VH CDR1, VH CDR2, and the sequence YLVY in VH FR3 may be involved in the binding of a small molecule agent to an antibody with conditional affinity provided herein. In some embodiments, the amino acid sequence YLVY in VH FR3 is important for the binding of the small molecule agent methotrexate to an antibody with conditional affinity provided herein, such that methotrexate more readily binds to the antibody if the antibody contains the sequence YLVY in the VH FR3 than if the antibody does not contain the sequence YLVY in the VH FR3.

In some embodiments, an antibody provided herein may contain the amino acid sequences CAA and YLVY (SEQ ID NO: 165) in the VH region. In some embodiments, both the CAA and YLVY sequences may be in the VH FR3 region. The CAA and YLVY sequences may be separated in the amino acid sequence of the VH FR3 region by other amino acids, or they may be contiguous. The CAA and YLVY sequences may be in either order in the amino acid sequence of the VH FR3 region.

In some embodiments, provided herein are antibodies having conditional affinity, wherein the antibody comprises at least one of the CDRs and optionally all of the CDRs of a clone as provided in the Examples section herein (e.g. VH CDRs 1, 2, and 3 and VL CDRs 1, 2, and 3 of a clone provided in, for example, Example 2 or Example 3 herein), or variants thereof.

In some embodiments, a VH region of an antibody with conditional affinity provided herein may comprise the amino acid sequence as shown in SEQ ID NO: 166.

Also provided herein are CDR portions of antibodies having conditional affinity. Determination of CDR regions is well within the skill of the art. It is understood that in some embodiments, CDRs can be a combination of the Kabat and Chothia CDR (also termed “combined CDRs” or “extended CDRs”). In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. In general, “conformational CDRs” include the residue positions in the Kabat CDRs and Vernier zones which are constrained in order to maintain proper loop structure for the antibody to bind a specific antigen. Determination of conformational CDRs is well within the skill of the art. In some embodiments, the CDRs are the Kabat CDRs. In other embodiments, the CDRs are the Chothia CDRs. In other embodiments, the CDRs are the extended, AbM, conformational, or contact CDRs. In other words, in embodiments with more than one CDR, the CDRs may be any of Kabat, Chothia, extended, AbM, conformational, contact CDRs or combinations thereof.

In some embodiments, an antibody having conditional affinity comprises three heavy chain CDRs of any clone shown in the Examples provided herein. In some embodiments, an antibody having conditional affinity comprises three light chain CDRs of any clone shown in the Examples provided herein. In some embodiments, an antibody having conditional affinity comprises three heavy chain CDRs and three light chain CDRs of any clone shown in the Examples provided herein.

In some embodiments, an antibody with conditional affinity may specifically bind an antigen on a mammalian cell. In some embodiments, an antibody with conditional affinity may specifically bind an antigen on a cancer cell. In some embodiments, an antibody with conditional affinity may specifically bind an antigen such as PD-1, PD-L1, CTLA-4, LAG-3, B7-H3, B7-H4, B7-DC (PD-L2), B7-H5, B7-H6, B7-H8, B7-H2, B7-1, B7-2, ICOS, ICOS-L, TIGIT, CD2, CD47, CD80, CD86, CD48, CD58, CD226, CD155, CD112, LAIR1, 2B4, BTLA, CD160, TIM1, TIM-3, TIM4, VISTA (PD-H1), OX40, OX40L, GITR, GITRL, CD70, CD27, 4-1BB, 4-BBL, DR3, TL1A, CD40, CD40L, CD30, CD30L, LIGHT, HVEM, SLAM (SLAMF1, CD150), SLAMF2 (CD48), SLAMF3 (CD229), SLAMF4 (2B4, CD244), SLAMF5 (CD84), SLAMF6 (NTB-A), SLAMCF7 (CS1), SLAMF8 (BLAME), SLAMF9 (CD2F), CD28, CEACAM1(CD66a), CEACAM3, CEACAM4, CEACAM5, CEACAM6, CEACAM7, CEACAM8, CEACAM1-3AS CEACAM3C2, CEACAM1-15, PSG1-11, CEACAM1-4C1, CEACAM1-4S, CEACAM1-4L, IDO, TDO, CCR2, CD39-CD73-adenosine pathway (A2AR), BTKs, TIKs, CXCR2, CCR4, CCR8, CCR5, VEGF pathway, CSF-1, or an innate immune response modulator.

In some embodiments, an antibody having conditional affinity comprises a full-length heavy chain, with or without the C-terminal lysine, and the full-length light chain.

Antibodies having conditional affinity can encompass, for example, monoclonal antibodies, antibody fragments (e.g., Fab, Fab′, F(ab′)₂, Fv, Fc, etc.), chimeric antibodies, bispecific antibodies, heteroconjugate antibodies, single chain (ScFv), antibody-drug conjugates (ADCs), fusion proteins comprising an antibody portion (e.g., a domain antibody), humanized antibodies, and any other modified configuration of the immunoglobulin molecule that comprises an antigen recognition site of the required specificity, including glycosylation variants of antibodies, amino acid sequence variants of antibodies, and covalently modified antibodies. The antibodies may be murine, rat, human, or any other origin (including chimeric or humanized antibodies). In some embodiments, the antibody is a monoclonal antibody. In some embodiments, the antibody is a human or humanized antibody.

Antibodies provided herein may be made by any method known in the art. General techniques for production of human and mouse antibodies are known in the art and/or are described herein.

Antibodies having affinity for an antigen can be identified or characterized using methods known in the art, for example, in assays whereby reduction, amelioration, or neutralization of biological activity of the antigen is detected and/or measured. In some embodiments, an antibody having affinity against an antigen is identified by incubating an antigen with an antibody having conditional affinity provided herein and optionally, a small molecule agent, and monitoring binding and/or attendant reduction or neutralization of a biological activity of the antigen. The binding assay may be performed with, e.g., purified polypeptide(s), or with cells naturally expressing (e.g., various strains), or transfected to express, antigen polypeptide(s). In one embodiment, the binding assay is a competitive binding assay, where the ability of a candidate antibody to compete with a known antibody having affinity for the antigen is evaluated. The assay may be performed in various formats, including the ELISA format. In some embodiments, an antibody having conditional affinity is identified by incubating a candidate antibody with an antigen of interest and optionally a small molecule agent, and monitoring binding.

Antibodies having conditional affinity provided herein may be characterized using methods known in the art. For example, one method is to identify an epitope to which an antibody binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, New York, 1999. In an additional example, epitope mapping can be used to determine the sequence to which an antibody having conditional affinity binds. Epitope mapping is commercially available from various sources, for example, Pepscan Systems (Edelhertweg 15, 8219 PH Lelystad, The Netherlands). The epitope can be a linear epitope, i.e., contained in a single stretch of amino acids, or a conformational epitope formed by a three-dimensional interaction of amino acids that may not necessarily be contained in a single stretch. Peptides of varying lengths (e.g., at least 4-6 amino acids long) can be isolated or synthesized (e.g., recombinantly) and used for binding assays with an antibody. In another example, the epitope to which an antibody with conditional affinity binds can be determined in a systematic screening by using overlapping peptides derived from the antigen sequence and determining binding by the antibody. According to the gene fragment expression assays, the open reading frame encoding the antigen is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments may then be determined by immunoprecipitation and gel electrophoresis. Certain epitopes can also be identified by using large libraries of random peptide sequences displayed on the surface of phage particles (phage libraries) or yeast (yeast display). Alternatively, a defined library of overlapping peptide fragments can be tested for binding to a test antibody in simple binding assays. In an additional example, mutagenesis of an antigen, domain swapping experiments and alanine scanning mutagenesis can be performed to identify residues required, sufficient, and/or necessary for epitope binding. For example, alanine scanning mutagenesis experiments can be performed using a mutant antigen in which various residues of an antigen polypeptide have been replaced with alanine. By assessing binding of the antibody to the mutant antigen protein, the importance of the particular antigen residues to the antibody with conditional affinity binding can be assessed.

Yet another method which can be used to characterize an antibody with conditional affinity provided herein is to use competition assays with other antibodies known to bind to the same antigen, to determine if the antibody having conditional affinity binds to the same epitope on the antigen as other antibodies. Competition assays are well known to those of skill in the art, including in an ELISA format.

### Libraries

Also provided herein is a library which contains polynucleotides which encode a heterogeneous population of antibodies, wherein the heterogeneous population of antibodies comprises at least an antibody with conditional affinity as provided herein. Typically, a library as provided herein contains a large number of different polynucleotides encoding a large number of different antibodies, and further encodes multiple different antibodies with conditional affinity as provided herein, including antibodies having specificity against different antigens. Libraries may be prepared and selected, for example, using phage display technology. In some embodiments, libraries may contain any antibody with conditional affinity as described elsewhere herein.

In some embodiments, a library as provided herein may be prepared in which many or all of the polynucleotides encoding antibodies contain sequences which permit their respective encoded antibody to bind to a small molecule agent of interest. For example, an antibody which binds to a small molecule agent of interest may be used as a starting point for the generation of a library provided herein. In some embodiments, an antibody which binds to a small molecule agent of interest and which is used as a starting point for the generation of a library is a VHH camelid antibody or portions thereof (e.g. one or more of the CDRs) which bind to a small molecule of interest. During the generation of the library, nucleotide sequences which encode amino acid sequences in the antibody important for interaction with the small molecule agent of interest may be preserved, while variability may be introduced into nucleotide sequences that encode amino acids that may be involved in antigen recognition but which are not important or required for interaction of the antibody with the small molecule agent of interest. Optionally, the VHH camelid antibody or portions thereof which bind to a small molecule of interest may be further modified to incorporate amino acids which facilitate interaction of the VHH-derived sequence with an antibody light chain, such that an antibodies containing a VH and VL region (e.g. a Fab, scFv, or IgG) may be generated from the library. By following this approach for library generation, a large number of different antibodies which bind to a particular small molecule agent of interest but which also have affinity for many different antigens may be generated. In addition, a library containing these types of sequences may be screened to identify antibodies that have affinity for a particular antigen of interest, and for which the affinity of the antibodies for the antigen is altered by the concentration condition (e.g. high concentration or low concentration) of the small molecule agent of interest. In some embodiments, a library provided herein may encode antibodies in any suitable format having a VH region and VL region, such as scFv, Fab, or IgG.

In some embodiments, a library provided herein may contain multiple polynucleotides (i.e. at least a first polynucleotide and a second polynucleotide, etc.). A polynucleotide of a library may encode, for example, a VH region, a VL region, or both a VH region and VL region of an antibody with conditional affinity provided herein. In some embodiments, at least a first polynucleotide and a second polynucleotide of the library each contain at least a first portion and a second portion, wherein the first portion of each of the first polynucleotide and the second polynucleotide contains the same nucleotide sequence, and wherein the second portion of first polynucleotide and second polynucleotide contain different nucleotide sequences. For example, in some embodiments, the first portion of a polynucleotide sequence encodes a polypeptide comprising a VH CDR1 and VH CDR2 sequence, the second portion of the polynucleotide sequence encodes polypeptide comprising a VH CDR3 sequence, and a first polynucleotide and second polynucleotide of the library encode the same VH CDR1 and VH CDR2 sequences, but encode different VH CDR3 sequences.

In some embodiments, a library provided herein comprises polynucleotides which encode a heterogeneous population of VH regions of antibodies, wherein the polynucleotides of the library each encode a VH that comprises the amino acid sequence of a CDR1 and CDR2 of SEQ ID NO. 1. Optionally, the polynucleotides of the library each encode a VH that comprises a CDR1 comprising the amino acid sequence shown in SEQ ID NO: 43 (RRSSRSWAMH), SEQ ID NO: 116 (RRSSRSW), or SEQ ID NO: 117 (SWAMH). Optionally, the polynucleotides of the library each encode a VH that comprises a CDR2 comprising the amino acid sequence shown in SEQ ID NO: 44 (VISYDGRLKYYADSVKGRF) or SEQ ID NO: 118 (SYDGRL). Optionally, the polynucleotides of the library each encode a VH region that comprises a FR3 comprising the amino acid sequence CAA. Optionally, the polynucleotides of the library each encode a VH region that comprises a FR3 comprising the amino acid sequence YLVY (SEQ ID NO: 165). Optionally, the polynucleotides of the library each encode a VH that comprises a CDR1 comprising the amino acid sequence shown in SEQ ID NO: 43 (RRSSRSWAMH), SEQ ID NO: 116 (RRSSRSW), or SEQ ID NO: 117 (SWAMH) and a CDR2 comprising the amino acid sequence shown in SEQ ID NO: 44 (VISYDGRLKYYADSVKGRF) or SEQ ID NO: 118 (SYDGRL). Optionally, the polynucleotides of the library each encode a VH region that comprises the amino acid sequence as shown in SEQ ID NO: 1. Optionally, the polynucleotides of the library each encode a VH region that comprises the amino acid sequence as shown in SEQ ID NO: 166. Optionally, the polynucleotides of any of the libraries as described above encode VH regions that contain different CDR3 sequences from each other (e.g. the polynucleotides of the library may, for example, all encode the same VH CDR1 and CDR2 sequence, but encode different VH CDR3 sequences).

Libraries provided herein may be prepared, for example, by the method described in Zhai W. et al, J. Mol. Biol. 412, 55-71 (2011), which is hereby incorporated by reference for all purposes. In some embodiments, a library may be prepared or screened according to methods as described in, e.g., U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; Fuchs et al. (1991), Biotechnology, 9:1369-1372; Hay et al., Hum. Antibod. Hybridomas, 3:81-85 (1992); Huse et al., Science, 246:1275-1281 (1989); McCafferty et al., Nature, 348:552-554 (1990); Griffiths et al., EMBO J, 12:725-734 (1993); Hawkins et al., J. Mol. Biol., 226:889-896 (1992); Clackson et al., Nature, 352:624-628 (1991); Gram et al., Proc. Natl. Acad. Sci. USA, 89:3576-3580 (1992); Garrad et al., Biotechnology, 9:1373-1377 (1991); Hoogenboom et al., Nuc Acid Res, 19:4133-4137 (1991); and Barbas et al., Proc. Natl. Acad. Sci. USA, 88:7978-7982 (1991). In some embodiments, cDNAs encoding heavy and light chains are independently supplied or linked to form Fv analogs for production in a phage library.

In some embodiments, a non-human animal amendable to genetic manipulation (e.g. mouse, rat) may be genetically engineered to contain genes encoding antibody sequences which contain a binding motif for a small molecule agent of interest, such that the animal can be immunized with an antigen of interest, and then screening can be performed for antibodies with conditional affinity from the animal, wherein the antibody with conditional affinity contains a binding motif for the small molecule agent of interest, and wherein the antibody binds to the antigen of interest with varying affinity, depending on the concentration of the small molecule agent of interest. For example, antibody genes which encode a VH region of an antibody which has a MTX binding-motif may be engineered into a mouse. The engineered mouse can then be immunized with an antigen of interest, and then antibodies from the engineered mouse can then be screened for antibodies which have MTX concentration-variable affinity for antigen of interest.

In some embodiments, sequences from an antibody isolated from an organism according to a process as provided herein (e.g. from a mouse as described above) may be combined with synthetic sequences. For example, DNA sequences encoding one or more CDRs from an antibody with conditional affinity obtained from a mouse as described above may combined with one or more CDRs or FRs that are synthetically derived.

In some embodiments, provided herein is a method of isolating an antibody with conditional affinity from a clonal library, the method including a) screening the library for clones which specifically bind to an antigen of interest and b) screening clones identified in step a) for clones which have variable affinity for the antigen of interest depending on the concentration of a small molecule agent of interest. In some embodiments, the clonal library used in this method is prepared using at least some polynucleotide sequences known to encode antibody sequences which specifically bind to the small molecule agent of interest.

In general, during screening of phage display libraries, genetic material from a clone of interest can be recovered and manipulated by standard techniques as known in the art and described in references provided herein. Multiple rounds of enrichment can increase the affinity of an antibody for an antigen or for the impact of a small molecule agent on the affinity of the antibody for an antigen. For example, general principles of phage display are described in U.S. Pat. Nos. 5,565,332; 5,580,717; 5,733,743; and 6,265,150; and Winter et al., Annu. Rev. Immunol. 12:433-455, 1994.

In some embodiments, phage display technology (McCafferty et al., Nature 348:552-553, 1990) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for review see, e.g., Johnson, Kevin S. and Chiswell, David J., Current Opinion in Structural Biology 3:564-571, 1993. Several sources of V-gene segments can be used for phage display. Clackson et al., Nature 352:624-628, 1991, isolated a diverse array of anti-oxazolone antibodies from a small random combinatorial library of V genes derived from the spleens of immunized mice. A repertoire of V genes from human donors can be constructed and antibodies to a diverse array of antigens (including self-antigens) can be isolated essentially following the techniques described by Mark et al., J. Mol. Biol. 222:581-597, 1991, or Griffith et al., EMBO J. 12:725-734, 1993. In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling.” (Marks et al., Bio/Technol. 10:779-783, 1992). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the pM-nM range. A strategy for making very large phage antibody repertoires (also known as “the mother-of-all libraries”) has been described by Waterhouse et al., Nucl. Acids Res. 21:2265-2266, 1993. Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable regions capable of restoring a functional antigen-binding site, i.e., the epitope governs (imprints) the choice of partner. When the process is repeated in order to replace the remaining rodent V domain, a human antibody is obtained (see PCT Publication No. WO 93/06213). Unlike traditional humanization of rodent antibodies by CDR grafting, this technique provides completely human antibodies, which have no framework or CDR residues of rodent origin.

### Chimeric Antigen Receptors and CAR-Expressing Immune Cells

In some embodiments, antibodies with conditional affinity as provided herein may be incorporated a chimeric antigen receptor (“CAR”). Nucleic acids encoding CARs containing an antibody with conditional affinity may be introduced into immune cells (e.g. T cells), to generate immune cells which express CARs. T cells which express CARs may be referred to as “CAR-T cells”.

Typically, a CAR contains an extracellular domain, a transmembrane domain, and an intracellular signaling domain. The extracellular domain contains at least an antigen binding region [e.g. an antigen binding fragment of a monoclonal antibody, such as a single-chain variable fragment (“scFv”)], and may also contain, for example, a spacer region and a signal peptide region. While the antigen binding region of a CAR (also referred to herein as the “antibody” of a CAR) is generally an scFv, other suitable antibody forms (e.g. a single domain antibody) are not excluded. The spacer region may, for example, facilitate the movement of the antigen binding domain and aid in the access of the antigen binding domain to the antigen, and the signal peptide region may contain a signal peptide which directs the extracellular orientation of the extracellular domain. In an extracellular domain containing a signal peptide region, an antigen binding region, and a spacer region, in some embodiments, the regions are ordered in the CAR polypeptide in the N-terminal to C-terminal direction as follows: signal peptide region - antigen binding region - spacer region. In some embodiments, the extracellular domain may contain a scFv, in which the scFv contains a VH region and a VL region of an antibody with conditional affinity as provided herein. The transmembrane domain may comprise a transmembrane domain of, for example, CD8, CD28, 4-1BB, CD3, CD4, CD8, FcγRl, NKG2D, FcεRlγ, ICOS, CTLA-4, PD-1, or VISTA. In some embodiments, the intracellular signaling domain may contain an activation signaling domain, and may contain, for example, an immunoreceptor tyrosine-based activation motif (“ITAM”) and/or a signaling domain of CD3 (e.g. CD3ζ), 4-1BB, CD28, ICOS, or OX40, or a combination thereof. In some embodiments, the intracellular signaling domain may contain an inhibitory signaling domain, and may contain, for example, an immunoreceptor tyrosine-based inhibition motif (“ITIM”) and/or a signaling domain of CTLA-4, PD-1, or VISTA, or a combination thereof.

Any immune cell capable of expressing heterologous DNAs can be used for the purpose of expressing the CAR of interest. In some embodiments, the immune cell is a T cell. In some embodiments, an immune cell can be derived from, for example and without limitation, a stem cell. The stem cells can be adult stem cells, non-human embryonic stem cells, more particularly non-human stem cells, cord blood stem cells, progenitor cells, bone marrow stem cells, induced pluripotent stem cells, totipotent stem cells or hematopoietic stem cells. Representative human cells are CD34+ cells. The immune cell may be, for example, a dendritic cell, a killer dendritic cell, a mast cell, a NK-cell, a B-cell or a T cell selected from the group consisting of inflammatory T-lymphocytes, cytotoxic T-lymphocytes, regulatory T-lymphocytes or helper T-lymphocytes. In some embodiments, the cell can be derived from the group consisting of CD4+ T-lymphocytes and CD8+ T-lymphocytes.

Binding of the antigen binding region of a CAR to an antigen may activate the immune cell containing the CAR. Specifically, following the binding of the antigen binding region of the CAR to an antigen, the intracellular signaling domain of the CAR is activated, resulting in activation of the immune cell and its immune response. The intracellular signaling domain has the ability to activate at least one of the normal effector functions of the immune cell in which the CAR is expressed. For example, the effector function of a T cell can be a cytolytic activity or helper activity, including the secretion of cytokines. Accordingly, an activated CAR T cell may have, for example, cytotoxic activity against a cell expressing an antigen recognized by the antibody with conditional affinity of a CAR provided herein.

Thus, for example, for a T cell containing a CAR containing an antibody with conditional affinity which specifically binds an antigen with higher affinity under a high agent concentration condition than a low agent concentration condition, if that CAR T cell is first in a low agent concentration condition and is then exposed to a high agent concentration condition (e.g. if that CAR T cell is in a subject, there is a baseline low agent concentration condition in the subject, and that subject is then administered the agent, leading to a high agent concentration condition in the subject), then the exposure of the CAR T cell to the high agent concentration will increase the binding of the CAR T cell to the antigen, and will lead to cytotoxic activity of the CAR T cell against the cell expressing the antigen. In another example, for a T cell containing a CAR containing an antibody with conditional affinity which specifically binds an antigen with lower affinity under a high agent concentration condition than a lower agent concentration condition, if that CAR T cell is exposed to a high agent concentration condition (e.g. if that CAR T cell is in a subject, there is a baseline low agent concentration condition in the subject, and that subject is then administered the agent), then the exposure of the CAR T cell to the high agent concentration will decrease the binding of the CAR T cell to the antigen, and will reduce or stop cytotoxic activity of the CAR T cell against the cell expressing the antigen.

Also, provided herein are isolated nucleic acids encoding a CAR containing an antibody with conditional affinity as provided herein, as well as related vectors [e.g. plasmids and viruses (e.g. lentivirus) containing nucleic acids encoding the CAR] and host cells containing the nucleic acid or vector. For example, CAR T cells expressing CARs containing antibodies with conditional affinity as provided herein may be used to treat a condition in a subject wherein the condition involves an antigen which may be targeted by the CAR T cell (e.g. an antigen on a malignant cell), and wherein it may be advantageous to conditionally target the antigen with the CAR T cell (i.e. to only have the CAR T cell bind to the antigen under certain conditions, such as a high agent concentration or low agent concentration). In an example, in an embodiment, a subject may have cancer and it may be advantageous to target an antigen on cancer cells in the subject with CAR T cells. However, it also may be advantageous to be able to control the affinity of the CAR T cells for the antigen on the cancer cells (e.g. to increase or decrease the binding of the CAR T cell to the antigen) in view of various factors (e.g. the health of the patient, the efficacy of the CAR T cells, etc.). Accordingly, CAR T cells as provided herein containing an antibody with conditional affinity may be provided to a subject, wherein the binding of the CAR T to the antigen may be regulated through a condition such as a small molecule agent concentration. In some embodiments, the agent may be a drug that can be administered to a patient. Also provided herein is a method of treating a subject having a disorder (e.g. a disease), the method including selecting a subject with a disorder that involves the expression of an antigen targeted by an antibody with conditional affinity provided herein, and administering to the subject a therapeutically effective amount of CAR T cells expressing a CAR containing an antibody with conditional affinity against the antigen.

In some embodiments, CARs and CAR T cells containing an antibody with conditional affinity as provided herein may be generally prepared as provided, for example, in Jackson et al, Nature Reviews Clinical Oncology, 13, 370-383 (2016); Maus et al, Blood, 123, 2625-2635 (2014); Dai et al, Journal of the National Cancer Institute, Vol 8, No. 7 (2016); Wang & Riviere, Molecular Therapy - Oncolytics, 3, 16015 (2016); Liechtenstein et al, Cancers (Basel), 5(3), 815-837 (2013), and in references provided therein. In some embodiments, a CAR or CAR-containing immune cell containing an antibody with conditional affinity as described herein may have any of the features and may be prepared by any of the methods described for CARs and CAR-containing immune cells provided in U.S. Pat. Application No. 15/085,317, filed Mar. 30, 2016, which is hereby incorporated by reference for all purposes.

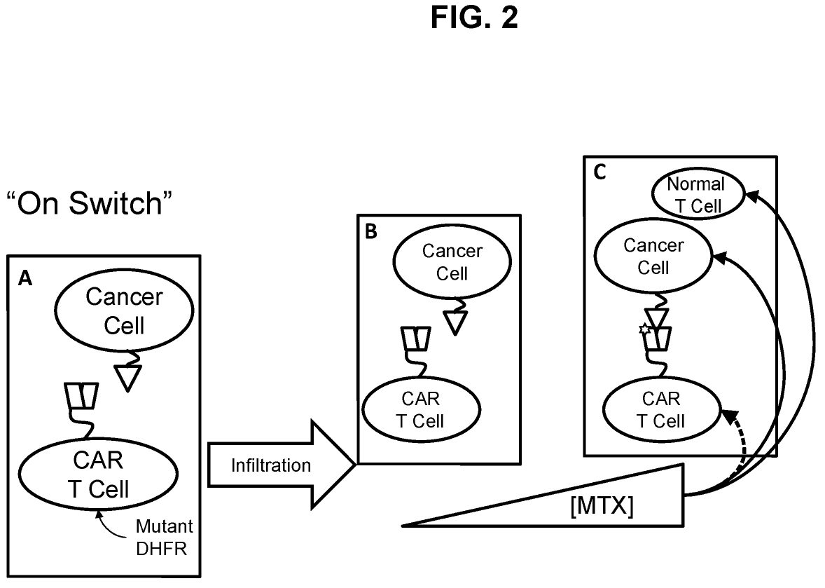

FIG. 2 provides a schematic involving exemplary possible features of a method provided herein involving a CAR T cell containing a CAR containing an antibody with conditional affinity as provided herein (e.g. a scFv), in which the antibody binds to an antigen with higher affinity in a high concentration condition of methotrexate as compared to a low concentration condition of methotrexate (i.e. in which the antibody of the CAR effectively has “on switch” which can be activated by methotrexate). In FIG. 2, the antibody of the CAR is depicted as a V-like shape at the end of strand protruding from a T cell; the strand and the V-like shape together represent the extracellular domain of the CAR (the strand represents a spacer region). The antibody of the CAR has conditional affinity for an antigen present on cancer cells; in FIG. 2, the antigen is depicted as a triangle shape on the outside of the cancer cells. The antigen may be any suitable antigen, preferably which is mostly or exclusively expressed on a cancer cell of interest.

In some embodiments of CAR T cells provided herein and as also depicted in FIG. 2, a CAR T cell may be engineered to additionally contain a mutated version of dihydrofolate reductase (“DHFR”) enzyme (“mutant DHFR”), in which the mutant DHFR is MTX-resistant (i.e. not inhibited by MTX or only weakly inhibited by MTX) but still catalytically active to reduce dihydrofolic acid to tetrahydrofolic acid. Mutant DHFR enzymes which are MTX-resistant but also catalytically active have been described, for example, in Schweitzer et al, J. Biol. Chem., 264, 20786-20795 (1989); Thompson and Freisheim, Biochemistry, 30, 8124-8130 (1991); Blakley et al, Advan. Expt. Med. Biol., 338, 473-479 (1993); Chunduru et al, J. Biol. Chem., 269, 9547-9555 (1994); Lewis et al, J. Biol. Chem., 270, 5057-5064 (1995); Ercikan-Abali et al, Mol. Pharmacol., 49, 430-437 (1996); Thillet et al, J. Biol. Chem., 263, 12500-12508 (1988); Volpato et al, J. Mol. Biol., 373(3), 599-611 (2007); Ercikan-Abali et al, Cancer Res, 56, 4142-4145 (1996); Flasshove et al, Leukemia, 9 (Suppl 1), S34-S37 (1995); Flasshove et al, Blood, 85, 566-574 (1995); and Jonnalagadda et al, Gene Therapy, 20, 853-860 (2013), which are each herein incorporated by reference for all purposes.

Turning again to FIG. 2, the CAR T cell depicted in FIG. 2 contains a chimeric antigen receptor containing an antibody with conditional affinity as provided herein (e.g. a scFv), in which the antibody binds to an antigen with higher affinity in a high concentration condition of methotrexate as compared to a low concentration condition of methotrexate, and in which the CAR T cell has also been engineered to express a mutant DHFR enzyme which is MTX-resistant. In some embodiments of methods provided herein, a CAR T cell having these characteristics may be administered to a subject in need thereof (e.g. a subject having cancer). Window “A” of FIG. 2 depicts a schematic of the interaction between a CAR T cell having the above characteristics and a cancer cell after administration of the CAR T cell to a subject having cancer, and in which methotrexate is in a low concentration condition in the subject (i.e. there is little or no methotrexate in the subject). Windows “B” and “C” of FIG. 2 show the same CAR T cell and cancer cell as in Window A, when methotrexate is in a low concentration (Window “B”) and when methotrexate is in a high concentration (Window “C”). As represented by the arrow between Window “A” and Window “B” of FIG. 2, upon administration of CAR T cells having the above characteristics to a subject, the CAR T cells may infiltrate to multiple sites within the subject. However, as depicted in Windows “A” and “B” of FIG. 2, as long as MTX is in a low concentration condition in the subject, the antibody with conditional affinity of the CAR T cell will have a low affinity for the antigen on the cancer cell, and thus the CAR T cell will have low or no binding to (or cytotoxic effect on) the cancer cell. In contrast, as depicted in FIG. 2, Window “C”, if MTX is administered to the subject so that the MTX reaches a high concentration condition, the antibody with conditional affinity of the CAR T cell will have a high affinity for the antigen on the cancer cell, and the CAR T cell will bind to and have a cytotoxic effect on the cancer cell.

In the above-described scenario of FIG. 2, the increase of MTX concentration in the subject may have various other effects in a subject. For example, increasing MTX concentration may result in any one or more of: i) a decrease in number of normal T cells or other lymphocytes, due to the cytotoxicity of MTX on normal lymphocytes; ii) a decrease in cancer cells, due to the cytotoxicity of MTX on cancer cells; iii) an increase in number of MTX-resistant cells (e.g. CAR T cells engineered with an MTX-resistant DHFR), due to the ability of MTX-resistant cells to proliferate in the presence of MTX. Thus, in the above scenario, for example, cancer cells may be killed by at least two separate mechanisms upon the increase in the concentration of MTX in the subject: i) increase in the affinity of CAR T cells for the cancer cells, such that the CAR T cells exert greater cytotoxic effect on the cancer cells, and ii) direct toxicity of the MTX on the cancer cells. Accordingly, use of CAR T cells as described in FIG. 2 with MTX may have improved therapeutic effects over MTX alone or CAR T cells alone.

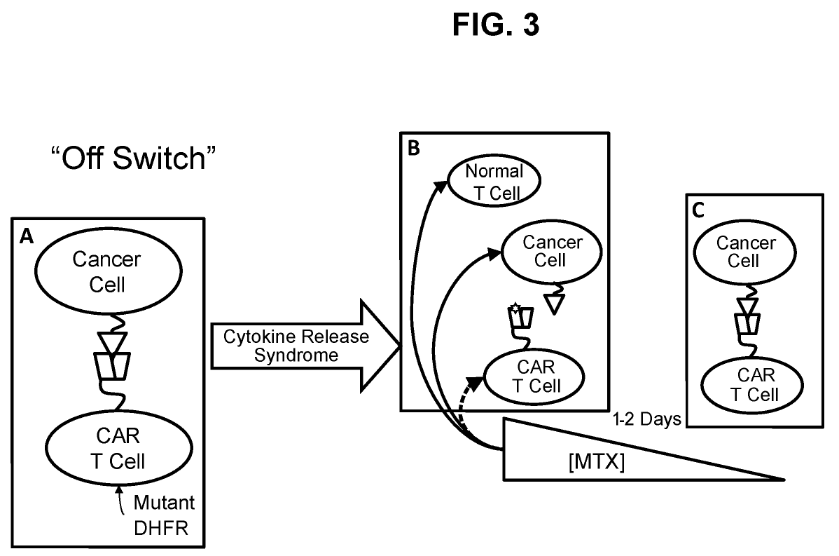

FIG. 3 provides a schematic involving exemplary possible features of another method provided herein involving an CAR T cell containing a chimeric antigen receptor containing an antibody with conditional affinity as provided herein (e.g. a scFv), in which the antibody binds to an antigen with higher affinity in a low concentration condition of methotrexate as compared to a high concentration condition of methotrexate (i.e. in which the antibody of the CAR effectively has “off switch” which can be turned off by methotrexate). In FIG. 3, the CAR elements are depicted in general as above in FIG. 2 (with the exception that the antibody binds to an antigen with higher affinity in a low concentration condition of methotrexate as compared to a high concentration condition of methotrexate), and the CAR T cells contain a MTX-resistant DHFR enzyme as described above in FIG. 2.

Thus, the CAR T cell depicted in FIG. 3 contains a chimeric antigen receptor containing an antibody with conditional affinity as provided herein (e.g. a scFv), in which the antibody binds to an antigen with higher affinity in a low concentration condition of methotrexate as compared to a high concentration condition of methotrexate, and in which the CAR T cell has also been engineered to express a mutant DHFR enzyme which is MTX-resistant. In some embodiments of methods provided herein, a CAR T cell having these characteristics may be administered to a subject in need thereof (e.g. a subject having cancer). Window “A” of FIG. 3 depicts a schematic of the interaction between a CAR T cell having the above characteristics and a cancer cell after administration of the CAR T cell to a subject having cancer, and in which methotrexate is in a low concentration condition in the subject (i.e. there is little or no methotrexate in the subject). Windows “B” and “C” of FIG. 3 show the same CAR T cell and cancer cell as in Window A, when methotrexate is in a high concentration (Window “B”) and when methotrexate is in a low concentration (Window “C”) (methotrexate is also in a low concentration condition in Window “A”). As depicted in Window “A” of FIG. 3, when a CAR T cell having the above characteristics is first administered to a subject (and when methotrexate is in a low concentration condition in the subject), the antibody with conditional affinity of the CAR T cell will have high affinity for the antigen on the cancer cell, and thus the CAR T cell will bind to and have a cytotoxic effect on the cancer cell. (Typically, binding of the CAR T cell to the antigen on the cancer cell will promote the release of cytokines from the CAR T cell.) While the cytotoxic effect of the CAR T cell on the cancer cell may produce a beneficial effect for the subject in terms of killing the cancer cell, the cytokines released from the CAR T cell and/or from other downstream activated immune cells may provoke a “cytokine release syndrome” in the subject (a systemic inflammatory response caused by the released cytokines), serious cases of which can have severe or fatal consequences for the subject (sometimes referred to as a “cytokine storm”). Accordingly, it may be beneficial to have a method to rapidly decrease the interaction of CAR T cells with cancer cells, in order to, for example, reduce or stop release of cytokines from the CAR T cells. As represented by the arrow between Window “A” and Window “B” of FIG. 3, upon administration of CAR T cells having the above characteristics to a subject, the CAR T cells may bind to the cancer cells, and this interaction may result in cytokine release syndrome in the subject. To ameliorate the effects the cytokine release syndrome, as shown in Window “B” of FIG. 3, if a cytokine release syndrome or other undesirable effects relating to the CAR T cell are detected, the subject can be administered MTX such that a high concentration condition is achieved, and so that the antibody with conditional affinity of the CAR T cell will have a low affinity for the antigen on the cancer cell, and thus the CAR T cell will have low or no binding to (or cytotoxic effect on) the cancer cell. As a result of the decreased binding of the CAR T cell to the cancer cell, the levels of cytokines in the subject may drop, and the cytokine release syndrome in the subject may be ameliorated. As depicted in Window “C” of FIG. 3, if the concentration of MTX in the subject is permitted to drop to a low concentration condition, the CAR T cells may again bind to the cancer cells, potentially leading again to increased levels of cytokines in the subject, and a cytokine release syndrome in the subject. However, the subject may be monitored for symptoms of a cytokine release syndrome or other effects resulting from CAR T cell activity, and administered a sufficient concentration of MTX such that a balance is achieved in the subject in which some CAR T cells bind to cancer cells (and thus beneficial effects are achieved in the subject) but some CAR T cells do not bind to cancer cells (and thus the side effects of T cell over-activity / cytokine release syndrome in the subject are avoided).

In the above-described scenario of FIG. 3, the increase of MTX concentration in the subject may have various other effects in a subject. For example, increasing MTX concentration may result in any one or more of: i) a decrease in number of normal T cells or other lymphocytes, due to the cytotoxicity of MTX on normal lymphocytes; ii) a decrease in cancer cells, due to the cytotoxicity of MTX on cancer cells; iii) an increase in number of MTX-resistant cells (e.g. CAR T cells engineered with an MTX-resistant DHFR), due to the ability of MTX-resistant cells to proliferate in the presence of MTX. In some embodiments related to FIG. 3 (e.g. if using a CAR T cell that does not contain MTX-resistant DHFR), the increase in MTX concentration may further decrease effects of a cytokine release syndrome mediated by the CAR T cells, by killing the CAR T cells.

In compositions and methods provided herein involving administration of MTX to a subject, optionally, the subject may also be administered a molecule which reduces the toxicity of MTX in the subject (for example, leucovorin / folinic acid), such that MTX-related toxicities in the subject, if present, may be reduced. Alternatively or additionally, for compositions and methods provided herein involving MTX, in some embodiments, MTX-derivatives may be used, in which the MTX-derivative still has the ability to affect the affinity of an antibody with conditional affinity provided herein for an antigen, but the MTX-derivative has reduced toxicity in a subject as compared to MTX.

In an alternative embodiment related to FIG. 3, in some embodiments, a CAR T cell as described generally above for FIG. 3 [i.e. that contains a chimeric antigen receptor which contains an antibody with conditional affinity as provided herein (e.g. a scFv), in which the antibody binds to an antigen with higher affinity in a low concentration condition of methotrexate as compared to a high concentration condition of methotrexate] may be administered to a subject when there is a high concentration condition of methotrexate in the subject. For example, the methotrexate may already be in the subject when the CAR T cells are administered to the subject, or it may be administered at the same time as administration of CAR T cells to the subject, or soon thereafter. Thus, in this embodiment, because a high concentration of methotrexate is present in the subject at or close to the time of administration of the CAR T cells to the subject, the CAR T cells will have a low affinity for the antigen on the cancer cell at or near the time of the initial administration of the CAR T cells to the subject. The subject may then be monitored for, for example, any adverse effects from the CAR T cells. These effects should be generally be small when there is a high concentration of methotrexate in the subject under these conditions, since the CAR T cells will have a low affinity for the antigen. Then, if the initial effect of the CAR T cells is acceptable to the subject, the concentration of methotrexate in the subject may be permitted to decrease (at least to some extent), in order to increase the affinity of the CAR T cell for the antigen on the cancer cell. Using this approach, the CAR T cells may be permitted to gradually increase affinity for the antigen on the cancer cell, so that the effects of the CAR T cells in the subject can be carefully regulated as needed, and undesirable effects from the CAR T cell binding to the antigen may be avoided or reduced.

While FIG. 2 and FIG. 3 depict exemplary CAR T cells which are engineered to express an MTX-resistant DHFR enzyme, also provided herein are similar CAR T cell embodiments in which the CAR T cells do not express an MTX-resistant DHFR enzyme. In addition, in some embodiments, an antibody with conditional affinity is provided herein, wherein the affinity of the antibody for the antigen is affected by a MTX-derivative which has reduced toxicity to T cells as compared to MTX, or no toxicity to T cells. Optionally, such MTX-derivatives also have little or no toxicity to cancer cells. Thus, such MTX-derivatives may affect the affinity of antibodies with conditional affinity provided herein for an antigen, without also being toxic (or having reduced toxicity) to T cells or other cells of the subject.

### Antibody Production and Modification

In some embodiments, an antibody having conditional affinity of interest may be sequenced and the polynucleotide sequence may then be cloned into a vector for expression or propagation. The sequence encoding the antibody of interest may be maintained in vector in a host cell and the host cell can then be expanded and frozen for future use.

In some embodiments, the polynucleotide sequence may be used for genetic manipulation to “humanize” the antibody or to improve the affinity, or other characteristics of the antibody. Antibodies may also be customized for use, for example, in dogs, cats, primate, equines and bovines.

DNA encoding monoclonal antibody clones may be isolated and sequenced using conventional procedures (e.g., by using obligonucleotide probes that are capable of binding specifically to genes encoding the heavy and light chains of the monoclonal antibodies). Once isolated, the DNA may be placed into expression vectors (such as expression vectors disclosed in PCT Publication No. WO 87/04462), which are then transfected into host cells such as E.coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA also may be modified, for example, by substituting the coding sequence for human heavy and light chain constant domains in place of the homologous murine sequences, Morrison et al., Proc. Nat. Acad. Sci. 81:6851, 1984, or by covalently joining to the immunoglobulin coding sequence all or part of the coding sequence for a non-immunoglobulin polypeptide. In that manner, “chimeric” or “hybrid” antibodies are prepared that have the binding specificity of an antibody having conditional affinity provided herein.

In some embodiments, fully human antibodies may be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse™ from Abgenix, Inc. (Fremont, CA) and HuMAb-Mouse® and TC Mouse™ from Medarex, Inc. (Princeton, NJ). Antibodies may be made recombinantly by first isolating the antibodies and antibody producing cells from host animals, obtaining the gene sequence, and using the gene sequence to express the antibody recombinantly in host cells (e.g., CHO cells). Another method which may be employed is to express the antibody sequence in plants (e.g., tobacco) or transgenic milk. Methods for expressing antibodies recombinantly in plants or milk have been disclosed. See, for example, Peeters, et al. Vaccine 19:2756, 2001; Lonberg, N. and D. Huszar Int. Rev. Immunol 13:65, 1995; and Pollock, et al., J Immunol Methods 231:147, 1999.

Antibody fragments can be produced by proteolytic or other degradation of the antibodies, by recombinant methods (i.e., single or fusion polypeptides) as described above or by chemical synthesis. Polypeptides of the antibodies, especially shorter polypeptides up to about 50 amino acids, are conveniently made by chemical synthesis. Methods of chemical synthesis are known in the art and are commercially available. For example, an antibody could be produced by an automated polypeptide synthesizer employing the solid phase method. See also, U.S. Pat. Nos. 5,807,715; 4,816,567; and 6,331,415.

Also provided herein are affinity matured embodiments of antibodies having conditional affinity. For example, affinity matured antibodies having conditional affinity can be produced from an initial version of an antibody having conditional affinity by procedures known in the art (Marks et al., 1992, Bio/Technology, 10:779-783; Barbas et al., 1994, Proc Nat. Acad. Sci, USA 91 :3809-3813 ; Schier et al., 1995, Gene, 169 :147-155 ; Yelton et al., 1995, J. Immunol., 155 :1994-2004 ; Jackson et al., 1995, J. Immunol., 154(7) :3310-9 ; Hawkins et al., 1992, J. Mol. Biol., 226:889-896; and PCT Publication No. WO2004/058184).

In some embodiments, the following methods may be used for adjusting the affinity of an antibody and for characterizing a CDR. One way of characterizing a CDR of an antibody and/or altering (such as improving) the binding affinity of a polypeptide, such as an antibody, termed “library scanning mutagenesis”. Generally, library scanning mutagenesis works as follows. One or more amino acid positions in the CDR are replaced with two or more (such as 3, 4, 5, 6, 7, 8, 9, 10, 11, 12, 13, 14, 15, 16, 17, 18, 19, or 20) amino acids using art recognized methods. This generates small libraries of clones (in some embodiments, one for every amino acid position that is analyzed), each with a complexity of two or more members (if two or more amino acids are substituted at every position). Generally, the library also includes a clone comprising the native (unsubstituted) amino acid. A small number of clones, e.g., about 20-80 clones (depending on the complexity of the library), from each library are screened for binding affinity to the target polypeptide (or other binding target), and candidates with increased, the same, decreased, or no binding are identified. Methods for determining binding affinity are well-known in the art. Binding affinity may be determined using, for example, Biacore™ surface plasmon resonance analysis, which detects differences in binding affinity of about 2-fold or greater, Kinexa® Biosensor, scintillation proximity assays, ELISA, ORIGEN® immunoassay, fluorescence quenching, fluorescence transfer, and/or yeast display. Binding affinity may also be screened using a suitable bioassay.

In some embodiments, every amino acid position in a CDR is replaced (in some embodiments, one at a time) with all 20 natural amino acids using art recognized mutagenesis methods (some of which are described herein). This generates small libraries of clones (in some embodiments, one for every amino acid position that is analyzed), each with a complexity of 20 members (if all 20 amino acids are substituted at every position).

In some embodiments, the library to be screened comprises substitutions in two or more positions, which may be in the same CDR or in two or more CDRs. Thus, the library may comprise substitutions in two or more positions in one CDR. The library may comprise substitution in two or more positions in two or more CDRs. The library may comprise substitution in 3, 4, 5, or more positions, said positions found in two, three, four, five or six CDRs. The substitution may be prepared using low redundancy codons. See, e.g., Table 2 of Balint et al., 1993, Gene 137(1):109-18.

The CDR may be heavy chain variable region (VH) CDR3 and/or light chain variable region (VL) CDR3. The CDR may be one or more of VH CDR1, VH CDR2, VH CDR3, VL CDR1, VL CDR2, and/or VL CDR3. The CDR may be a Kabat CDR, a Chothia CDR, an extended CDR, an AbM CDR, a contact CDR, or a conformational CDR.

Candidates with improved binding may be sequenced, thereby identifying a CDR substitution mutant which results in improved affinity (also termed an “improved” substitution). Candidates that bind may also be sequenced, thereby identifying a CDR substitution which retains binding.

Multiple rounds of screening may be conducted. For example, candidates (each comprising an amino acid substitution at one or more position of one or more CDR) with improved binding are also useful for the design of a second library containing at least the original and substituted amino acid at each improved CDR position (i.e., amino acid position in the CDR at which a substitution mutant showed improved binding). Preparation, and screening or selection of this library is discussed further below.

Library scanning mutagenesis also provides a means for characterizing a CDR, in so far as the frequency of clones with improved binding, the same binding, decreased binding or no binding also provide information relating to the importance of each amino acid position for the stability of the antibody-antigen complex. For example, if a position of the CDR retains binding when changed to all 20 amino acids, that position is identified as a position that is unlikely to be required for antigen binding. Conversely, if a position of CDR retains binding in only a small percentage of substitutions, that position is identified as a position that is important to CDR function. Thus, the library scanning mutagenesis methods generate information regarding positions in the CDRs that can be changed to many different amino acids (including all 20 amino acids), and positions in the CDRs which cannot be changed or which can only be changed to a few amino acids.

Candidates with improved affinity may be combined in a second library, which includes the improved amino acid, the original amino acid at that position, and may further include additional substitutions at that position, depending on the complexity of the library that is desired, or permitted using the desired screening or selection method. In addition, if desired, adjacent amino acid position can be randomized to at least two or more amino acids. Randomization of adjacent amino acids may permit additional conformational flexibility in the mutant CDR, which may in turn, permit or facilitate the introduction of a larger number of improving mutations. The library may also comprise substitution at positions that did not show improved affinity in the first round of screening.

The second library is screened or selected for library members with improved and/or altered binding affinity using any method known in the art, including screening using Kinexa™ biosensor analysis, and selection using any method known in the art for selection, including phage display, yeast display, and ribosome display.

The above techniques may also be used to adjust the affinity of an antibody having conditional affinity provided herein for a small molecule agent and/or the specificity of an antibody having conditional affinity for a particular small molecule agent (e.g. to select for antibodies which only bind to a single particular small molecule agent and which have little or no cross-reactivity with closely related small molecule agents). In some embodiments, the above techniques may be used to simultaneously adjust the affinity of an antibody for an antigen and to adjust the specificity and/or affinity of the antibody to a small molecule agent.

Embodiments provided herein encompass modifications to the variable regions and CDRs of antibodies with conditional affinity provided in the Examples. For example, embodiments provided herein include antibodies comprising functionally equivalent variable regions and CDRs which do not significantly affect their properties as well as variants which have enhanced or decreased activity and/or affinity. Examples of modified polypeptides include polypeptides with conservative substitutions of amino acid residues, one or more deletions or additions of amino acids which do not significantly deleteriously change the functional activity, or which mature (enhance) the affinity of the polypeptide for its ligand, or use of chemical analogs.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to an epitope tag. Other insertional variants of the antibody molecule include the fusion to the N- or C-terminus of the antibody of an enzyme or a polypeptide which increases the half-life of the antibody in the blood circulation.

Substitution variants have at least one amino acid residue in the antibody molecule removed and a different residue inserted in its place. The sites of greatest interest for substitutional mutagenesis include the hypervariable regions, but framework alterations are also contemplated. Conservative substitutions are shown in Table 1 under the heading of “conservative substitutions.” If such substitutions result in a change in biological activity, then more substantial changes, denominated “exemplary substitutions” in Table 1, or as further described below in reference to amino acid classes, may be introduced and the products screened.

TABLE 1

Amino Acid Substitutions

| | | |
|---|---|---|
| Original Residue | Conservative Substitutions | Exemplary Substitutions |
| Ala (A) | Val | Val; Leu; Ile |
| Arg I | Lys | Lys; Gln; Asn |
| Asn (N) | Gln | Gln; His; Asp, Lys; Arg |
| Asp (D) | Glu | Glu; Asn |
| Cys I | Ser | Ser; Ala |
| Gln (Q) | Asn | Asn; Glu |
| Glu I | Asp | Asp; Gln |
| Gly (G) | Ala | Ala |
| His (H) | Arg | Asn; Gln; Lys; Arg |
| Ile (I) | Leu | Leu; Val; Met; Ala; Phe; Norleucine |
| Leu (L) | Ile | Norleucine; Ile; Val; Met; Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile ; Leu ; Met; Phe ; Ala ; Norleucine |

Substantial modifications in the biological properties of the antibody are accomplished by selecting substitutions that differ significantly in their effect on maintaining (a) the structure of the polypeptide backbone in the area of the substitution, for example, as a β-sheet or helical conformation, (b) the charge or hydrophobicity of the molecule at the target site, or (c) the bulk of the side chain. Naturally occurring residues are divided into groups based on common side-chain properties:
(1) Non-polar: Norleucine, Met, Ala, Val, Leu, Ile;
(2) Polar without charge: Cys, Ser, Thr, Asn, Gln;
(3) Acidic (negatively charged): Asp, Glu;
(4) Basic (positively charged): Lys, Arg;
(5) Residues that influence chain orientation: Gly, Pro; and
(6) Aromatic: Trp, Tyr, Phe, His.

Non-conservative substitutions are made by exchanging a member of one of these classes for another class.

One type of substitution, for example, that may be made is to change one or more cysteines in the antibody, which may be chemically reactive, to another residue, such as, without limitation, alanine or serine. For example, there can be a substitution of a non-canonical cysteine. The substitution can be made in a CDR or framework region of a variable domain or in the constant region of an antibody. In some embodiments, the cysteine is canonical. Any cysteine residue not involved in maintaining the proper conformation of the antibody also may be substituted, generally with serine, to improve the oxidative stability of the molecule and prevent aberrant cross-linking. Conversely, cysteine bond(s) may be added to the antibody to improve its stability, particularly where the antibody is an antibody fragment such as an Fv fragment.

The antibodies may also be modified, e.g. in the variable domains of the heavy and/or light chains, e.g., to alter a binding property of the antibody. Changes in the variable region can alter binding affinity and/or specificity. In some embodiments, no more than one to five conservative amino acid substitutions are made within a CDR domain. In other embodiments, no more than one to three conservative amino acid substitutions are made within a CDR domain. For example, a mutation may be made in one or more of the CDR regions to increase or decrease the K_{D} of the antibody for an antigen, to increase or decrease k_{off}, or to alter the binding specificity of the antibody. Techniques in site-directed mutagenesis are well-known in the art. See, e.g., Sambrook et al. and Ausubel et al., supra.

A modification or mutation may also be made in a framework region or constant region to increase the half-life of an antibody with conditional affinity. See, e.g., PCT Publication No. WO 00/09560. A mutation in a framework region or constant region can also be made to alter the immunogenicity of the antibody, to provide a site for covalent or non-covalent binding to another molecule, or to alter such properties as complement fixation, FcR binding and antibody-dependent cell-mediated cytotoxicity. In some embodiments, no more than one to five conservative amino acid substitutions are made within the framework region or constant region. In other embodiments, no more than one to three conservative amino acid substitutions are made within the framework region or constant region. According to the invention, a single antibody may have mutations in any one or more of the CDRs or framework regions of the variable domain or in the constant region.

Modifications also include glycosylated and nonglycosylated polypeptides, as well as polypeptides with other post-translational modifications, such as, for example, glycosylation with different sugars, acetylation, and phosphorylation. Antibodies are glycosylated at conserved positions in their constant regions (Jefferis and Lund, 1997, Chem. Immunol. 65:111-128; Wright and Morrison, 1997, TibTECH 15:26-32). The oligosaccharide side chains of the immunoglobulins affect the protein’s function (Boyd et al., 1996, Mol. Immunol. 32:1311-1318; Wittwe and Howard, 1990, Biochem. 29:4175-4180) and the intramolecular interaction between portions of the glycoprotein, which can affect the conformation and presented three-dimensional surface of the glycoprotein (Jefferis and Lund, supra; Wyss and Wagner, 1996, Current Opin. Biotech. 7:409-416). Oligosaccharides may also serve to target a given glycoprotein to certain molecules based upon specific recognition structures. Glycosylation of antibodies has also been reported to affect antibody-dependent cellular cytotoxicity (ADCC). In particular, antibodies produced by CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al., 1999, Nature Biotech. 17:176-180).

Other methods of modification include using coupling techniques known in the art, including, but not limited to, enzymatic means, oxidative substitution and chelation. Modifications can be used, for example, for attachment of labels for immunoassay. Modified polypeptides are made using established procedures in the art and can be screened using standard assays known in the art, some of which are described below and in the Examples.

In some embodiments, the Fc can be human IgG₁, IgG₂, IgG₃, or human IgG₄. In some embodiments, the antibody comprises a constant region of IgG₄ comprising the following mutations (Armour et al., 2003, Molecular Immunology 40 585-593): E233F234L235 to P233V234A235 (IgG_{4ΔC}), in which the numbering is with reference to wild type IgG₄. In yet another embodiment, the Fc is human IgG₄ E233F234L235 to P233V234A235 with deletion G236 (IgG_{4Δb}). In some embodiments the Fc is any human IgG₄ Fc (IgG₄, IgG_{4Δb} or IgG_{4Δc}) containing hinge stabilizing mutation S228 to P228 (Aalberse et al., 2002, Immunology 105, 9-19). In other embodiments, the Fc can be human IgG₂ containing the mutation A330P331 to S330S331 (IgG_{2Δa}), in which the amino acid residues are numbered with reference to the wild type IgG₂ sequence. Eur. J. Immunol., 1999, 29:2613-2624.

In some embodiments, the antibody comprises a modified constant region that has increased or decreased binding affinity to a human Fc gamma receptor, is immunologically inert or partially inert, e.g., does not trigger complement mediated lysis, does not stimulate antibody-dependent cell mediated cytotoxicity (ADCC), or does not activate microglia; or has reduced activities (compared to the unmodified antibody) in any one or more of the following: triggering complement mediated lysis, stimulating ADCC, or activating microglia. Different modifications of the constant region may be used to achieve optimal level and/or combination of effector functions. See, for example, Morgan et al., Immunology 86:319-324, 1995; Lund et al., J. Immunology 157:4963-9 157:4963-4969, 1996; Idusogie et al., J. Immunology 164:4178-4184, 2000; Tao et al., J. Immunology 143: 2595-2601, 1989; and Jefferis et al., Immunological Reviews 163:59-76, 1998. In some embodiments, the constant region is modified as described in Eur. J. Immunol., 1999, 29:2613-2624; PCT Publication No. WO99/058572.

In some embodiments, an antibody constant region can be modified to avoid interaction with Fc gamma receptor and the complement and immune systems. The techniques for preparation of such antibodies are described in WO 99/58572. For example, the constant region may be engineered to more resemble human constant regions to avoid immune response if the antibody is used in clinical trials and treatments in humans. See, e.g., U.S. Pat. Nos. 5,997,867 and 5,866,692.

Other antibody modifications include antibodies that have been modified as described in PCT Publication No. WO 99/58572. These antibodies comprise, in addition to a binding domain directed at the target molecule, an effector domain having an amino acid sequence substantially homologous to all or part of a constant region of a human immunoglobulin heavy chain. These antibodies are capable of binding the target molecule without triggering significant complement dependent lysis, or cell-mediated destruction of the target. In some embodiments, the effector domain is capable of specifically binding FcRn and/or FcyRllb. These are typically based on chimeric domains derived from two or more human immunoglobulin heavy chain CH2 domains. Antibodies modified in this manner are particularly suitable for use in chronic antibody therapy, to avoid inflammatory and other adverse reactions to conventional antibody therapy.

In some embodiments, the antibody comprises a modified constant region that has increased binding affinity for FcRn and/or an increased serum half-life as compared with the unmodified antibody.

In a process known as “germlining”, certain amino acids in the VH and VL sequences can be mutated to match those found naturally in germline VH and VL sequences. In particular, the amino acid sequences of the framework regions in the VH and VL sequences can be mutated to match the germline sequences to reduce the risk of immunogenicity when the antibody is administered. Germline DNA sequences for human VH and VL genes are known in the art (see e.g., the “Vbase” human germline sequence database; see also Kabat, E. A., et al., 1991, Sequences of Proteins of Immunological Interest, Fifth Edition, U.S. Department of Health and Human Services, NIH Publication No. 91-3242; Tomlinson et al., 1992, J. Mol. Biol. 227:776-798; and Cox et al., 1994, Eur. J. Immunol. 24:827-836).

Another type of amino acid substitution that may be made is to remove potential proteolytic sites in the antibody. Such sites may occur in a CDR or framework region of a variable domain or in the constant region of an antibody. Substitution of cysteine residues and removal of proteolytic sites may decrease the risk of heterogeneity in the antibody product and thus increase its homogeneity. Another type of amino acid substitution is to eliminate asparagine-glycine pairs, which form potential deamidation sites, by altering one or both of the residues. In another example, the C-terminal lysine of the heavy chain of an antibody with conditional affinity provided herein can be cleaved. In various embodiments, the heavy and light chains of an antibody with conditional affinity may optionally include a signal sequence.

Once DNA fragments encoding the VH and VL segments of the present invention are obtained, these DNA fragments can be further manipulated by standard recombinant DNA techniques, for example to convert the variable region genes to full-length antibody chain genes, to Fab fragment genes, or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat, E. A., et al., 1991, Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG₁, IgG₂, IgG₃, IgG₄, IgA, IgE, IgM or IgD constant region, but most preferably is an IgG₁ or IgG₂ constant region. The IgG constant region sequence can be any of the various alleles or allotypes known to occur among different individuals, such as Gm(1), Gm(2), Gm(3), and Gm(17). These allotypes represent naturally occurring amino acid substitution in the IgG1 constant regions. For a Fab fragment heavy chain gene, the VH-encoding DNA can be operatively linked to another DNA molecule encoding only the heavy chain CH1 constant region. The CH1 heavy chain constant region may be derived from any of the heavy chain genes.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al., 1991, Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region. The kappa constant region may be any of the various alleles known to occur among different individuals, such as Inv(1), Inv(2), and Inv(3). The lambda constant region may be derived from any of the three lambda genes.

To create a scFv gene, the VH- and VL-encoding DNA fragments are operatively linked to another fragment encoding a flexible linker such that the VH and VL sequences can be expressed as a contiguous single-chain protein, with the VL and VH regions joined by the flexible linker (See e.g., Bird et al., 1988, Science 242:423-426; Huston et al., 1988, Proc. Natl. Acad. Sci. USA 85:5879-5883; McCafferty et al., 1990, Nature 348:552-554. An example of a linking peptide is GGGGSGGGGSGGGGS (SEQ ID NO: 2), which bridges approximately 3.5 nm between the carboxy terminus of one variable region and the amino terminus of the other variable region. Linkers of other sequences have been designed and used (Bird et al., 1988, supra). Linkers can in turn be modified for additional functions, such as attachment of drugs or attachment to solid supports. The single chain antibody may be monovalent, if only a single VH and VL are used, bivalent, if two VH and VL are used, or polyvalent, if more than two VH and VL are used. Bispecific or polyvalent antibodies may be generated that bind specifically to different antigens of interest. The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

Other forms of single chain antibodies, such as diabodies, are also encompassed. Diabodies are bivalent, bispecific antibodies in which VH and VL are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al., 1993, Proc. Natl. Acad Sci. USA 90:6444-6448; Poljak, R. J., et al., 1994, Structure 2:1121-1123).

Heteroconjugate antibodies, comprising two covalently joined antibodies, are also within the scope of the invention. Such antibodies have been used to target immune system cells to unwanted cells (U.S. Pat. No. 4,676,980), and for treatment of HIV infection (PCT Publication Nos. WO 91/00360 and WO 92/200373; EP 03089). Heteroconjugate antibodies may be made using any convenient cross-linking methods. Suitable cross-linking agents and techniques are well known in the art, and are described in U.S. Pat. No. 4,676,980.

Chimeric or hybrid antibodies also may be prepared in vitro using known methods of synthetic protein chemistry, including those involving cross-linking agents. For example, immunotoxins may be constructed using a disulfide exchange reaction or by forming a thioether bond. Examples of suitable reagents for this purpose include iminothiolate and methyl-4-mercaptobutyrimidate.

Embodiments provided herein also include fusion proteins comprising one or more fragments or regions from the antibodies disclosed herein. In some embodiments, a fusion antibody may be made that comprises all or a portion of an antibody with conditional affinity linked to another polypeptide. In another embodiment, only the variable domains of an antibody with conditional affinity are linked to the polypeptide. In another embodiment, only the CDRs of an antibody with conditional affinity are linked to the polypeptide. In another embodiment, the VH domain of an antibody with conditional affinity is linked to a first polypeptide, while the VL domain of an antibody with conditional affinity is linked to a second polypeptide that associates with the first polypeptide in a manner such that the VH and VL domains can interact with one another to form an antigen binding site. In another embodiment, the VH domain is separated from the VL domain by a linker such that the VH and VL domains can interact with one another. The VH-linker- VL antibody is then linked to a polypeptide of interest. In addition, fusion antibodies can be created in which two (or more) single-chain antibodies are linked to one another. This is useful if one wants to create a divalent or polyvalent antibody on a single polypeptide chain, or if one wants to create a bispecific antibody.

In some embodiments, a fusion polypeptide is provided that comprises at least 10, 15, 20, or 25 contiguous amino acids of a CDR of an antibody with conditional affinity described in the Examples provided herein. In some embodiments, a fusion polypeptide comprises VH CDR3 and/or VL CDR3 of an antibody with conditional affinity described in the Examples provided herein. For purposes of this disclosure, a fusion protein contains one or more antibodies and another amino acid sequence to which it is not attached in the native molecule, for example, a heterologous sequence or a homologous sequence from another region. Exemplary heterologous sequences include, but are not limited to a “tag” such as a FLAG tag or a 6His tag. Tags are well known in the art.

A fusion polypeptide can be created by methods known in the art, for example, synthetically or recombinantly. Typically, the fusion proteins of this invention are made by preparing and expressing a polynucleotide encoding them using recombinant methods described herein, although they may also be prepared by other means known in the art, including, for example, chemical synthesis.

In other embodiments, other modified antibodies may be prepared using antibody with conditional affinity-encoding nucleic acid molecules. For instance, “Kappa bodies” (III et al., 1997, Protein Eng. 10:949-57), “Minibodies” (Martin et al., 1994, EMBO J. 13:5303-9), “Diabodies” (Holliger et al., supra), or “Janusins” (Traunecker et al., 1991, EMBO J. 10:3655-3659 and Traunecker et al., 1992, Int. J. Cancer (Suppl.) 7:51-52) may be prepared using standard molecular biological techniques following the teachings of the specification.

For example, bispecific antibodies, monoclonal antibodies that have binding specificities for at least two different antigens, can be prepared using the antibodies disclosed herein. Methods for making bispecific antibodies are known in the art (see, e.g., Suresh et al., 1986, Methods in Enzymology 121:210). For example, bispecific antibodies or antigen-binding fragments can be produced by fusion of hybridomas or linking of Fab′ fragments. See, e.g., Songsivilai & Lachmann, 1990, Clin. Exp. Immunol. 79:315-321, Kostelny et al., 1992, J. Immunol. 148:1547-1553. Traditionally, the recombinant production of bispecific antibodies was based on the coexpression of two immunoglobulin heavy chain-light chain pairs, with the two heavy chains having different specificities (Millstein and Cuello, 1983, Nature 305, 537-539). In addition, bispecific antibodies may be formed as “diabodies” or “Janusins.” In some embodiments, the bispecific antibody binds to two different epitopes of an antigen. In some embodiments, the modified antibodies described above are prepared using one or more of the variable domains or CDR regions from an antibody with conditional affinity provided herein.

Also provided herein are compositions comprising antibodies conjugated (for example, linked) to an agent that facilitate coupling to a solid support (such as biotin or avidin). For simplicity, reference will be made generally to antibodies with the understanding that these methods apply to the antibodies with conditional affinity described herein. Conjugation generally refers to linking these components as described herein. The linking (which is generally fixing these components in proximate association at least for administration) can be achieved in any number of ways. For example, a direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

The antibodies can be bound to many different carriers. Carriers can be active and/or inert. Examples of well-known carriers include polypropylene, polystyrene, polyethylene, dextran, nylon, amylases, glass, natural and modified celluloses, polyacrylamides, agaroses and magnetite. The nature of the carrier can be either soluble or insoluble. Those skilled in the art will know of other suitable carriers for binding antibodies, or will be able to ascertain such, using routine experimentation. In some embodiments, the carrier comprises a moiety that targets a particular organ or tissue type.

An antibody or polypeptide provided herein may be linked to a labeling agent such as a fluorescent molecule, a radioactive molecule or any others labels known in the art. Labels are known in the art which generally provide (either directly or indirectly) a signal.

In some embodiments, an antibody with conditional affinity provided herein may be used to purify an antigen of interest (e.g. during a protein purification process). The conditionally-specific antibody may be at a first time incubated with a material containing the antigen of interest and under conditions in which the antibody binds to the antigen with high affinity (for example, if the antibody binds to antigen with high affinity under low agent concentration conditions, then at the first time, the antibody is incubated with the antigen in a low agent concentration condition), such that an antibody-antigen complex is formed. Optionally, the antibody-antigen complex may be washed with suitable buffers in order to separate the antigen from remainder of the material that originally contained the antigen. Then, at a second time, the antibody may be exposed to conditions in which the antibody binds to the antigen with low affinity, such that the antibody-antigen complex disassociates.

In some embodiments, antibodies may be made using hybridoma technology. It is contemplated that any mammalian subject including humans or antibody producing cells therefrom can be manipulated to serve as the basis for production of mammalian, including human, hybridoma cell lines. The route and schedule of immunization of the host animal are generally in keeping with established and conventional techniques for antibody stimulation and production, as further described herein. Typically, the host animal is inoculated intraperitoneally, intramuscularly, orally, subcutaneously, intraplantar, and/or intradermally with an amount of immunogen, including as described herein.

Hybridomas can be prepared from the lymphocytes and immortalized myeloma cells using the general somatic cell hybridization technique of Kohler, B. and Milstein, C., 1975, Nature 256:495-497 or as modified by Buck, D. W., et al., In Vitro, 18:377-381, 1982. Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce monoclonal antibodies provided herein. The hybridomas or other immortalized B-cells are expanded and subcloned, if desired, and supernatants are assayed for anti-immunogen activity by conventional immunoassay procedures (e.g., radioimmunoassay, enzyme immunoassay, or fluorescence immunoassay).

Hybridomas that produce antibodies provided herein may be grown in vitro or in vivo using known procedures. Monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity, if present, can be removed, for example, by running the preparation over adsorbents made of the immunogen attached to a solid phase and eluting or releasing the desired antibodies off the immunogen.

Immunization of a host animal with an antigen, or a fragment containing a target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N=C=NR, where R and R¹ are different alkyl groups, can yield a population of antibodies (e.g., monoclonal antibodies).

### Polynucleotides, Vectors, and Host Cells

Also provided herein are polynucleotides encoding any of the antibodies with conditional affinity provided herein, including antibody fragments, antigen binding regions, and modified antibodies described herein, such as, e.g., antibodies having impaired effector function. In another aspect, provided herein is a method of making any of the polynucleotides described herein. Polynucleotides can be made and expressed by procedures known in the art. Accordingly, the invention includes polynucleotides or compositions, including pharmaceutical compositions, comprising polynucleotides, encoding any of the antibodies with conditional affinity provided herein. Also provided are polynucleotides encoding any of the libraries described herein. Polynucleotides complementary to any such sequences are also encompassed. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

In some embodiments, provided herein is a polynucleotide sequence encoding one or more CDRs of an antibody having conditional affinity (e.g. as provided in the Examples). The sequence encoding an antibody of interest may be maintained in a vector in a host cell and the host cell can then be expanded and frozen for future use. Vectors (including expression vectors) and host cells are further described herein.

To express an antibody with conditional affinity provided herein, DNA fragments encoding VH and VL regions can first be obtained using any of the methods described above. Various modifications, e.g. mutations, deletions, and/or additions can also be introduced into the DNA sequences using standard methods known to those of skill in the art. For example, mutagenesis can be carried out using standard methods, such as PCR-mediated mutagenesis, in which the mutated nucleotides are incorporated into the PCR primers such that the PCR product contains the desired mutations or site-directed mutagenesis.

Variants of polynucleotides that encode an antibody having conditional affinity provided herein are also encompassed. Generally, polynucleotide variants may contain one or more substitutions, additions, deletions and/or insertions such that the immunoreactivity of the encoded polypeptide is not diminished, relative to the original starting molecule. The effect on the immunoreactivity of the encoded polypeptide may generally be assessed as described herein. Variants preferably exhibit at least about 70% identity, more preferably, at least about 80% identity, yet more preferably, at least about 90% identity, and most preferably, at least about 95% identity to a polynucleotide sequence that encodes an antibody provided herein.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, or 40 to about 50, in which a sequence may be compared to a reference sequence of the same number of contiguous positions after the two sequences are optimally aligned.

Optimal alignment of sequences for comparison may be conducted using the MegAlign® program in the Lasergene® suite of bioinformatics software (DNASTAR®, Inc., Madison, WI), using default parameters. This program embodies several alignment schemes described in the following references: Dayhoff, M.O., 1978, A model of evolutionary change in proteins – Matrices for detecting distant relationships. In Dayhoff, M.O. (ed.) Atlas of Protein Sequence and Structure, National Biomedical Research Foundation, Washington DC Vol. 5, Suppl. 3, pp. 345-358; Hein J., 1990, Unified Approach to Alignment and Phylogenes pp. 626-645 Methods in Enzymology vol. 183, Academic Press, Inc., San Diego, CA; Higgins, D.G. and Sharp, P.M., 1989, CABIOS 5:151-153; Myers, E.W. and Muller W., 1988, CABIOS 4:11-17; Robinson, E.D., 1971, Comb. Theor. 11:105; Santou, N., Nes, M., 1987, Mol. Biol. Evol. 4:406-425; Sneath, P.H.A. and Sokal, R.R., 1973, Numerical Taxonomy the Principles and Practice of Numerical Taxonomy, Freeman Press, San Francisco, CA; Wilbur, W.J. and Lipman, D.J., 1983, Proc. Natl. Acad. Sci. USA 80:726-730.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e. the window size) and multiplying the results by 100 to yield the percentage of sequence identity.

It will be appreciated by those of ordinary skill in the art that, as a result of the degeneracy of the genetic code, there are many nucleotide sequences that encode a polypeptide as described herein. Nonetheless, polynucleotides that vary due to differences in codon usage are specifically contemplated by the present invention.

The polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al., 1989.

Alternatively, PCR allows reproduction of DNA sequences. PCR technology is well known in the art and is described in U.S. Pat. Nos. 4,683,195, 4,800,159, 4,754,065 and 4,683,202, as well as PCR: The Polymerase Chain Reaction, Mullis et al. eds., Birkauswer Press, Boston, 1994.

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., 1989, supra, for example.

Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors are further provided. Expression vectors generally are replicable polynucleotide constructs that contain a polynucleotide according to the invention. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

Also provided are host cells comprising any of the polynucleotides described herein. Any host cells capable of over-expressing heterologous DNAs can be used for the purpose of isolating the genes encoding the antibody or expressing the polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E.coli or B.subtillis) and yeast (such as S.cerevisae, S.pombe; or K.lactis). An expression vector can be used to direct expression of an antibody with conditional affinity. In some embodiments, expression vectors to obtain expression of an exogenous protein in vivo may be used. See, e.g., U.S. Pat. Nos. 6,436,908; 6,413,942; and 6,376,471. Administration of expression vectors includes local or systemic administration, including injection, oral administration, particle gun or catheterized administration, and topical administration.

Targeted delivery of therapeutic compositions containing an expression vector, or subgenomic polynucleotides can also be used. Receptor-mediated DNA delivery techniques are described in, for example, Findeis et al., Trends Biotechnol., 1993, 11:202; Chiou et al., Gene Therapeutics: Methods And Applications Of Direct Gene Transfer, J.A. Wolff, ed., 1994; Wu et al., J. Biol. Chem., 1988, 263:621; Wu et al., J. Biol. Chem., 1994, 269:542; Zenke et al., Proc. Natl. Acad. Sci. USA, 1990, 87:3655; Wu et al., J. Biol. Chem., 1991, 266:338. Therapeutic compositions containing a polynucleotide may be administered in a range of about 100 ng to about 200 mg of DNA for local administration in a gene therapy protocol. Concentration ranges of about 500 ng to about 50 mg, about 1 µg to about 2 mg, about 5 µg to about 500 µg, and about 20 µg to about 100 µg of DNA can also be used during a gene therapy protocol. The therapeutic polynucleotides and polypeptides can be delivered using gene delivery vehicles. The gene delivery vehicle can be of viral or non-viral origin (see generally, Jolly, Cancer Gene Therapy, 1994, 1:51; Kimura, Human Gene Therapy, 1994, 5:845; Connelly, Human Gene Therapy, 1995, 1:185; and Kaplitt, Nature Genetics, 1994, 6:148). Expression of such coding sequences can be induced using endogenous mammalian or heterologous promoters. Expression of the coding sequence can be either constitutive or regulated.

### Compositions

In some embodiments, provided herein are pharmaceutical compositions comprising an effective amount of an antibody with conditional affinity described herein. Examples of such compositions, as well as how to formulate, are also provided herein. In some embodiments, the composition comprises one or more antibodies with conditional affinity. In some embodiments, the antibody with conditional affinity is a human antibody. In other embodiments, the antibody with conditional affinity is a humanized antibody. In some embodiments, the antibody with conditional affinity comprises a constant region that is capable of triggering a desired immune response, such as antibody-mediated lysis or ADCC. In other embodiments, the antibody with conditional affinity comprises a constant region that does not trigger an unwanted or undesirable immune response, such as antibody-mediated lysis or ADCC.

It is understood that the compositions can comprise more than one antibody with conditional affinity (e.g., a mixture of antibodies with conditional affinity that recognize different epitopes of an antigen, optionally where two or more or all of the antibodies have variable antigen affinity depending on the concentration of a particular small molecule agent).

Compositions provided herein can further comprise pharmaceutically acceptable carriers, excipients, or stabilizers (Remington: The Science and practice of Pharmacy 20^{th} Ed., 2000, Lippincott Williams and Wilkins, Ed. K. E. Hoover), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Antibodies having conditional affinity and compositions thereof can also be used in conjunction with, or administered separately, simultaneously, or sequentially with other agents that serve to enhance and/or complement the effectiveness of the agents.

Also provided are compositions, including pharmaceutical compositions, comprising any of the polynucleotides provided herein. In some embodiments, the composition comprises an expression vector comprising a polynucleotide encoding an antibody with conditional affinity as described herein.

### Methods for Preventing or Treating Conditions

The antibodies provided herein are useful in various applications including, but are not limited to, therapeutic treatment methods and diagnostic treatment methods.

In one aspect, provided herein is a method for treating a cancer. In some embodiments, the method of treating a cancer in a subject comprises administering to the subject in need thereof an effective amount of a composition (e.g., pharmaceutical composition) comprising an antibody with conditional affinity or lymphocytes containing an antibody with conditional affinity (e.g. a CAR T cell), as described herein. As used herein, cancers include, but are not limited to bladder cancer, breast cancer, cervical cancer, choriocarcinoma, colon cancer, esophageal cancer, gastric cancer, glioblastoma, glioma, brain tumor, head and neck cancer, kidney cancer, lung cancer, oral cancer, ovarian cancer, pancreatic cancer, prostate cancer, liver cancer, uterine cancer, bone cancer, leukemia, lymphoma, sarcoma, blood cancer, thyroid cancer, thymic cancer, eye cancer, and skin cancer. In some embodiments, provided is a method of inhibiting tumor growth or progression in a subject, comprising administering to the subject in need thereof an effective amount of a composition comprising the an antibody with conditional affinity or lymphocytes containing an antibody with conditional affinity (e.g. a CAR T cell) as described herein. In other embodiments, provided is a method of inhibiting metastasis of cancer cells in a subject, comprising administering to the subject in need thereof an effective amount of a composition comprising any of the antibodies with conditional affinity or lymphocytes containing an antibody with conditional affinity (e.g. a CAR T cell) as described herein. In other embodiments, provided is a method of inducing regression of a tumor in a subject, comprising administering to the subject in need thereof an effective amount of a composition comprising an antibody with conditional affinity or lymphocytes containing an antibody with conditional affinity (e.g. a CAR T cell), as described herein.

In another aspect, provided is a method of detecting, diagnosing, and/or monitoring a cancer. For example, antibodies with conditional affinity as described herein can be labeled with a detectable moiety such as an imaging agent and an enzyme-substrate label. The antibodies as described herein can also be used for in vivo diagnostic assays, such as in vivo imaging (e.g., PET or SPECT), or a staining reagent.

In some embodiments, the methods described herein further comprise a step of treating a subject with an additional form of therapy. In some embodiments, the additional form of therapy is an additional anti-cancer therapy including, but not limited to, chemotherapy, radiation, surgery, hormone therapy, and/or additional immunotherapy.

With respect to all methods described herein, reference to antibodies with conditional affinity also includes compositions comprising antibodies and one or more additional agents. These compositions may further comprise, for example, small molecule agents or suitable excipients, such as pharmaceutically acceptable excipients including buffers, which are well known in the art. Methods provided herein can be used alone or in combination with other methods of treatment.

An antibody with conditional affinity can be administered to a subject via any suitable route. It should be apparent to a person skilled in the art that the examples described herein are not intended to be limiting but to be illustrative of the techniques available. Accordingly, in some embodiments, the antibody with conditional affinity is administered to a subject in accord with known methods, such as intravenous administration, e.g., as a bolus or by continuous infusion over a period of time, by intramuscular, intraperitoneal, intracerebrospinal, transdermal, subcutaneous, intra-articular, sublingually, intrasynovial, via insufflation, intrathecal, oral, inhalation or topical routes. Administration can be systemic, e.g., intravenous administration, or localized. Commercially available nebulizers for liquid formulations, including jet nebulizers and ultrasonic nebulizers are useful for administration. Liquid formulations can be directly nebulized and lyophilized powder can be nebulized after reconstitution. Alternatively, an antibody with conditional affinity can be aerosolized using a fluorocarbon formulation and a metered dose inhaler, or inhaled as a lyophilized and milled powder.

In some embodiments, an antibody with conditional affinity is administered via site-specific or targeted local delivery techniques. Examples of site-specific or targeted local delivery techniques include various implantable depot sources of the antibody with conditional affinity or local delivery catheters, such as infusion catheters, indwelling catheters, or needle catheters, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Various formulations of an antibody with conditional affinity may be used for administration. In some embodiments, an antibody with conditional affinity may be administered neat. In some embodiments, an antibody with conditional affinity and a pharmaceutically acceptable excipient may be in various formulations. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 20^{th} Ed. Mack Publishing, 2000.

In some embodiments, these agents are formulated for administration by injection (e.g., intraperitoneally, intravenously, subcutaneously, intramuscularly, etc.). Accordingly, these agents can be combined with pharmaceutically acceptable vehicles such as saline, Ringer’s solution, dextrose solution, and the like. The particular dosage regimen, i.e., dose, timing and repetition, may depend on the particular individual and that individual’s medical history.

An antibody with conditional affinity can be administered using any suitable method, including by injection (e.g., intraperitoneally, intravenously, subcutaneously, intramuscularly, etc.). An antibody with conditional affinity can also be administered topically or via inhalation, as described herein. In some embodiments, for administration of antibodies having conditional affinity, an initial candidate dosage can be about 2 mg/kg. For the purpose of the present invention, a typical daily dosage might range from about any of 3 µg/kg to 30 µg/kg to 300 µg/kg to 3 mg/kg, to 30 mg/kg, to 100 mg/kg or more, depending on the factors mentioned above. For example, dosage of about 1 mg/kg, about 2.5 mg/kg, about 5 mg/kg, about 10 mg/kg, and about 25 mg/kg may be used. In some embodiments, a fixed dose of antibody having conditional affinity may be administered to subjects (i.e. the dose is independent of subject mass). For repeated administrations over several days or longer, depending on the condition, the treatment is sustained until a desired suppression of symptoms occurs or until sufficient therapeutic levels are achieved, for example, to reduce symptoms associated with cancer. The progress of this therapy may be monitored by conventional techniques and assays. The dosing regimen (including the antibody with conditional affinity) can vary over time. In some embodiments, the dosing regimen may also include administering to the subject the small molecule agent which affects the affinity of the antibody with conditional affinity for an antigen.

In some embodiments, the appropriate dosage of an antibody with conditional affinity may depend on the antibody with conditional affinity (or compositions thereof) employed, the type and severity of symptoms to be treated, whether the agent is administered for preventive or therapeutic purposes, previous therapy, the patient’s clinical history and response to the agent, the patient’s clearance rate for the administered agent, and the discretion of the attending physician. Typically the clinician will administer an antibody with conditional affinity, and optionally, a small molecule agent, until a dosage is reached that achieves the desired result. Dose and/or frequency can vary over course of treatment. Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host’s immune system. Frequency of administration may be determined and adjusted over the course of therapy, and is generally, but not necessarily, based on treatment and/or suppression and/or amelioration and/or delay of symptoms. Alternatively, sustained continuous release formulations of antibodies having conditional affinity may be appropriate. Various formulations and devices for achieving sustained release are known in the art.

In one embodiment, dosages for an antibody with conditional affinity, and optionally, dosage for a corresponding small molecule agent may be determined empirically in individuals who have been given one or more administration(s) of an antibody with conditional affinity. Individuals are given incremental dosages of an antibody with conditional affinity, and optionally increasing or decreasing amounts of a small molecule agent. To assess efficacy, an indicator of the disease can be followed. In some embodiments, administration of a small molecule agent to a subject may increase the efficacy of an antibody with conditional affinity in the subject (i.e. by increasing the affinity of the antibody for an antigen). In other embodiments, administration of a small molecule to a subject may decrease the efficacy of an antibody with conditional affinity in the subject (i.e. by decreasing the affinity of the antibody for an antigen).

In some embodiments, an antibody with conditional affinity may be administered in combination with the administration of one or more additional therapeutic agents. These include, but are not limited to, the administration of a chemotherapeutic agent, a vaccine, a CAR-T cell-based therapy, radiotherapy, a cytokine therapy, a vaccine, an inhibitor of other immunosuppressive pathways, an inhibitors of angiogenesis, a T cell activator, an inhibitor of a metabolic pathway, an mTOR inhibitor, an inhibitor of an adenosine pathway, a tyrosine kinase inhibitor including but not limited to inlyta, ALK inhibitors and sunitinib, a BRAF inhibitor, an epigenetic modifier, an inhibitor of Treg cells and/or of myeloid-derived suppressor cells, a JAK inhibitor, a STAT inhibitor, a cyclin-dependent kinase inhibitor, a biotherapeutic agent (including but not limited to antibodies to VEGF, VEGFR, EGFR, Her2/neu, other growth factor receptors, CD20, CD40, CD-40L, CTLA-4, OX-40, 4-1BB, and ICOS), an immunogenic agent (for example, attenuated cancerous cells, tumor antigens, antigen presenting cells such as dendritic cells pulsed with tumor derived antigen or nucleic acids, immune stimulating cytokines (for example, IL-2, IFNα2, GM-CSF), and cells transfected with genes encoding immune stimulating cytokines such as but not limited to GM-CSF). Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gamma1l and calicheamicin phil1, see, e.g., Agnew, Chem. Intl. Ed. Engl., 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin, and deoxydoxorubicin), pegylated liposomal doxorubicin, epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′,2″-trichlorotriethylamine; trichothecenes (especially T-2 toxin, verracurin A, roridin A and anguidine); vindesine; dacarbazine; mannomustine; mitobronitol; mitolactol; pipobroman; gacytosine; arabinoside (“Ara-C”); cyclophosphamide; thiotepa; paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; and pharmaceutically acceptable salts, acids or derivatives of any of the above.

### Formulations

Therapeutic formulations of an antibody with conditional affinity used as provided herein are prepared for storage by mixing an antibody having the desired degree of purity with optional pharmaceutically acceptable carriers, excipients or stabilizers (Remington, The Science and Practice of Pharmacy 20^{th} Ed. Mack Publishing, 2000), in the form of lyophilized formulations or aqueous solutions. Acceptable carriers, excipients, or stabilizers are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Liposomes containing an antibody with conditional affinity may be prepared by methods known in the art, such as described in Epstein, et al., Proc. Natl. Acad. Sci. USA 82:3688 (1985); Hwang, et al., Proc. Natl Acad. Sci. USA 77:4030 (1980); and U.S. Pat. Nos. 4,485,045 and 4,544,545. Liposomes with enhanced circulation time are disclosed in U.S. Patent No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The active ingredients may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy 20^{th} Ed. Mack Publishing (2000).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT ™ (injectable microspheres composed of lactic acid-glycolic acid copolymer and leuprolide acetate), sucrose acetate isobutyrate, and poly-D-(-)-3-hydroxybutyric acid.

The formulations to be used for in vivo administration must be sterile. This is readily accomplished by, for example, filtration through sterile filtration membranes. Therapeutic antibody with conditional affinity compositions are generally placed into a container having a sterile access port, for example, an intravenous solution bag or vial having a stopper pierceable by a hypodermic injection needle.

The compositions provided herein may be in unit dosage forms such as tablets, pills, capsules, powders, granules, solutions or suspensions, or suppositories, for oral, parenteral or rectal administration, or administration by inhalation or insufflation.

In some embodiments, an antibody with conditional affinity provided herein is prepared in an injectable form (e.g. for intravenous or subcutaneous injection), and a small molecule agent which affects the affinity of the antibody for an antigen is prepared for enteral administration (e.g. in tablet or liquid form).

For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules.

### Kits

The invention also provides kits comprising any of the antibodies with conditional affinity described herein. Kits provided herein may include one or more containers comprising an antibody with conditional affinity described herein, and optionally, a corresponding small molecule agent (i.e. a small molecule agent that can affect the affinity of the antibody for an antigen) and instructions for use in accordance with any of the methods of the invention described herein. Generally, these instructions comprise a description of administration of the antibody with conditional affinity, and optionally, a small molecule agent, for the above described therapeutic treatments. In some embodiments, kits are provided for producing a single-dose administration unit. In certain embodiments, the kit can contain both a first container having a dried protein and a second container having an aqueous formulation. In certain embodiments, kits containing single and multi-chambered pre-filled syringes (e.g., liquid syringes and lyosyringes) are included. In some embodiments, the kit can contain a container containing a small molecule agent. In some embodiments, the small molecule agent may be in a separate container from the container containing the antibody with conditional affinity.

The instructions relating to the use of an antibody with conditional affinity and optionally, a small molecule agent, generally include information as to dosage, dosing schedule, and route of administration for the intended treatment (including dosing information for both the antibody with conditional affinity and for the small molecule agent). The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). In some embodiments, a kit also comprises a small molecule agent which can alter the affinity of the antibody with conditional affinity in the kit for an antigen.

Kits may optionally provide additional components such as buffers and interpretive information. Normally, the kit comprises a container and a label or package insert(s) on or associated with the container.

Incorporated by reference herein for all purposes is the content of U.S. Provisional Pat. Application Nos. 62/484,776 (filed Apr. 12, 2017) and 62/637,077 (filed Mar. 1, 2018).

### General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M.J. Gait, ed., 1984); Methods in Molecular Biology, Humana Press; Cell Biology: A Laboratory Notebook (J.E. Cellis, ed., 1998) Academic Press; Animal Cell Culture (R.I. Freshney, ed., 1987); Introduction to Cell and Tissue Culture (J.P. Mather and P.E. Roberts, 1998) Plenum Press; Cell and Tissue Culture: Laboratory Procedures (A. Doyle, J.B. Griffiths, and D.G. Newell, eds., 1993-1998) J. Wiley and Sons; Methods in Enzymology (Academic Press, Inc.); Handbook of Experimental Immunology (D.M. Weir and C.C. Blackwell, eds.); Gene Transfer Vectors for Mammalian Cells (J.M. Miller and M.P. Calos, eds., 1987); Current Protocols in Molecular Biology (F.M. Ausubel et al., eds., 1987); PCR: The Polymerase Chain Reaction, (Mullis et al., eds., 1994); Current Protocols in Immunology (J.E. Coligan et al., eds., 1991); Short Protocols in Molecular Biology (Wiley and Sons, 1999); Immunobiology (C.A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J.D. Capra, eds., Harwood Academic Publishers, 1995), as well as in subsequent editions and corresponding websites of the above references, as applicable.

### Examples

The following examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

### Example 1: Generation of a Library for Screening for Antibodies with Methotrexate-Modulated Conditional Affinity

This example illustrates the generation of a synthetic human single-chain variable fragment (scFv) library which may be used to screen for scFvs which bind to antigens with methotrexate-modulated affinity. In other words, the library can be used to screen for scFvs which have a relatively high affinity or relatively low affinity for an antigen of interest under a high concentration or low concentration of methotrexate, as desired, and selected for by the screening conditions. The heavy chain sequence from a methotrexate binding antibody (“MTX-VH”) and three light chain germline frameworks were selected as the backbone for the synthetic human antibody library designed and synthesized in a manner similar to that previously described [Zhai W. et al, J. Mol. Biol. 412, 55-71 (2011)]. In brief, VK1-39, VK3-20 and VL1-47 were chosen based on their frequency in the memory compartment of the immune repertoire obtained from 218 donors, canonical complementarity determining region (CDR) structural diversity, and stability. Representative crystal structures for each germline found in the Protein Databank Bank (PDB) were analyzed in conjunction with the immune repertoire data to select CDR positions to diversify based on their structural proximity to the antigen and abundance of naturally observed amino acid variability, respectively. No diversity was introduced into CDR1 or CDR2 of the MTX-VH. A unique germline specific design was generated for light chain CDR1 and CDR2 based on the natural amino acid distribution found in the entire repertoire of 218 donors based on non-redundant clones to avoid biasing the results based on clonal expansion. Unique cassettes for CDR3 of lengths 8-10 for VK1-39, lengths 8-10 for VK3-20, lengths 10-12 for VL1-47 and lengths 6-17 for MTX-VH were chosen and account for 89%, 82%, 85% and 81% of the unique sequences found in the repertoire analyzed, respectively. The library was synthesized using the Slonomics DNA synthesis technology, cloned into a scFv phagemid then transformed into E.coli resulting in a library of 1.5 × 10¹¹ transformants.

The amino acid sequence encoded by the VH region of the library is:

QVQLVESGGGLVQAGGSLRLSCAASRRSSRSWAMHWVRQAPGKGLEWVAV
ISYDGRLKYYADSVKGRFTISRDNAEYLVYLQMNSLRAEDTAVYYCAA
[X₆₋₁₇] WGQGTLVTVSS

, in which X is any amino acid, and there may be any of 6, 7, 8, 9, 10, 11, 12, 13, 14, 15, 16, or 17 “X” amino acids (SEQ ID NO: 166). Also, CDR1, CDR2, and CDR3 sequences are in sequential order and are underlined (Kabat) and in bold (Chothia). As depicted in the sequence, the CDR3 region of the VH region (i.e. the X amino acids) is highly variable in the number, sequence, and type of amino acid present.

### Example 2: Selection of Anti-CD33 Antibodies Having Conditional Affinity

This Example describes selection of anti-CD33 antibodies having conditional affinity.

Antibodies toward human CD33 (also known as “Siglec-3”; UniProt Accession Number: P20138) that exhibit diminished binding affinities in the presence methotrexate (“MTX”) were identified from the phage displayed libraries described in Example 1 by subjecting the libraries to 5 rounds of panning. The first 4 rounds of panning used biotinylated CD33 antigen captured on streptavidin or neutravidin coated 96-well plates. The 5^{th} round of panning was performed in solution by preincubating the phage displayed antibodies with biotinylated antigen then capturing them on a neutravidin coated 96-well plate. 1536 clones were randomly chosen from the 4^{th} and 5^{th} round of panning against the CD33 antigen and their binding specificity determined by phage ELISA. Clones that specifically bound to CD33 were sequenced and all unique clones were subjected to a phage competition ELISA to assess their relative affinities to CD33 and sensitivity to MTX by measuring their binding strengths in the presence or absence of 10 µM (micromolar) MTX (i.e. in 0 or 10 µM MTX). Clones that demonstrated a decrease in binding in the presence of MTX were expressed as scFv-Fc fusions and their CD33 binding kinetics in the presence and absence of 10 µM MTX were measured using a biosensor.

All binding kinetics analysis was performed on label-free Biacore surface plasmon biosensors (Biacore T200™ from GE Life Sciences) at 37° C. in 10 mM Hepes pH 7.4, 150 mM NaCl, 0.05% Tween-20 (HBST+), with or without 10 µM MTX. The Biacore data were processed in Biacore T200 Evaluation Software, and the data were double-referenced (Myszka, 1999, J Mol Recognit 12(5):279-284) and fit globally to a simple Langmuir with mass transport model to determine the equilibrium dissociation constant, K_{D}, from the ratio of the kinetic rate constants (K_{D} = k_{off}/kₒₙ). In some cases the binding of antigen was very low (or not detectable) in the presence of 10 µM MTX and as a result kinetic rate constants could not be accurately determined. In these cases, a K_{D} (or K_{D} limit) was estimated based on the binding response at the highest analyte concentration tested or based on the typical limit of detection for a Biacore T200.

Table 2 provides kinetic data for various anti-CD33 clones having conditional affinity for CD33 identified through this screening process. As shown in Table 2, numerous clones were selected which have an antibody-antigen K_{D} in 10 µM MTX / antibody-antigen K_{D} in 0 µM MTX ratio of greater than, for example, 2, 3, 4, 5, 10, 20, or 50. For example, the K_{D} of the clone P02_D08 to CD33 in 10 µM MTX is 705 nM whereas in 0 µM MTX the K_{D} is 100 nM. Thus, the ratio of the K_{D} in 10 µM MTX / the K_{D} in 0 µM MTX is: 705 nM / 100 nM, and equals ~ 7.1. In other words, the affinity of the P02_D08 clone for CD33 is 7.1 times greater in 0 µM MTX than in 10 µM MTX. Also of note, for certain clones, the antibody-antigen K_{D} in 10 µM MTX is greater than a given limit (e.g. “>10929”). Thus, this value indicates a very weak interaction (as described by the limit) or a case where there is no antibody-antigen binding.

The majority of the anti-CD33 clones analyzed have less than 2-fold difference in binding affinity at 0 µM MTX vs. 10 µM MTX; most of these clones are not included in Table 2. However, for reference purposes, Table 2 also includes kinetic data for 2 of these types of anti-CD33 clones (P01_C05 and P02_C02).

During screening assays for anti-CD33 antibodies with conditional affinity, at least 1 clone was also identified that has an increased affinity for CD33 in the presence of 10 µM MTX. Kinetic information for this clone (P01_B02) is also provided in Table 2 below. As shown in Table 2, the K_{D} of the P01_B02 antibody-CD33 interaction in the presence of 0 µM MTX is 19 nM, whereas in the presence of 10 µM MTX, the K_{D} of the antibody-CD33 interaction is 1.6 nM. Thus, the ratio of K_{D} with MTX / without MTX is about 0.1 (i.e. the antibody has about 10 times greater affinity for CD33 in the presence of 10 µM MTX as compared to in 0 µM MTX).

TABLE 2

| | Kinetics toward antigen with 0 µM MTX | | | Kinetics toward antigen with 10 µM MTX | | | |
|---|---|---|---|---|---|---|---|
| Clone | kon (1/Ms) | koff (1/s) | K_{D} (nM) | kon (1/Ms) | koff (1/s) | K_{D} (nM)* | Ratio: K_{D} with MTX / K_{D} without MTX |
| P01_A09 | 1.1E+05 | 1.7E-02 | 154 | - | - | 1305 | 8.5 |
| P01_F05 | 7.1E+04 | 1.0E-02 | 143 | - | - | >13208 | >92.5 |
| P02_A10 | 8.4E+04 | 4.4E-02 | 523 | - | - | >9022 | >17.3 |
| P02_D08 | 6.1E+04 | 6.1E-03 | 100 | 8.8E+04 | 6.2E-02 | 705 | 7.1 |
| P02_E11 | 5.8E+04 | 3.8E-03 | 66 | - | - | 14594 | 221 |
| P02_H01 | 2.8E+04 | 3.7E-02 | 1321 | - | - | 4339 | 3.3 |
| P03_A01 | 5.0E+04 | 5.7E-03 | 115 | - | - | 4271 | 37.0 |
| P03_G12 | 7.1E+04 | 9.4E-03 | 132 | - | - | >10929 | >83.0 |
| P04_C09 | 1.6E+05 | 1.8E-03 | 12 | 4.4E+04 | 8.9E-03 | 204.6 | 17.7 |
| P07_C04 | 9.5E+04 | 2.9E-03 | 31 | 1.7E+05 | 2.5E-02 | 143.6 | 4.7 |
| P08_C08 | 6.3E+04 | 4.5E-03 | 72 | 9.4E+04 | 5.1E-02 | 547 | 7.6 |
| P16_E06 | 1.2E+05 | 1.1E-02 | 92 | 7.5E+04 | 8.7E-02 | 1167 | 12.7 |
| P02_B03 | 1.4E+05 | 2.3E-02 | 170 | - | - | >340 | >2.0 |
| P02_D09 | 4.0E+04 | 1.8E-02 | 456 | - | - | >9295 | >20.4 |
| P02_E08 | 4.7E+04 | 1.0E-02 | 211 | - | - | >726 | >3.4 |
| P03_H04 | 7.2E+04 | 2.6E-02 | 366 | - | - | >15731 | >43.0 |
| P04_C05 | 8.3E+04 | 2.1E-03 | 25 | 4.5E+04 | 1.3E-02 | 297.8 | 12 |
| P01_C05 | 7.6E+04 | 6.6E-03 | 86 | 1.0E+05 | 4.5E-03 | 44.7 | 0.5 |
| P02_C02 | 9.2E+04 | 2.0E-02 | 213 | 1.1E+05 | 2.5E-02 | 220.7 | 1 |
| P01_B02 | 1.4E+05 | 2.6E-03 | 19 | 3.9E+05 | 6.0E-04 | 1.6 | 0.1 |

“*”: For interactions where kₒₙ and k_{off} are reported, the K_{D} was calculated using k_{off}/kₒₙ. For weak interactions in which kinetic rate constants could not be accurately determined (e.g. some of the cases of antigen-antibody interaction in the presence of 10 µM MTX), estimates of the K_{D} were made from the binding response at the highest antigen concentration tested, or based on the typical limit of detection for a Biacore T200.

The amino acid sequences of the VH and VL regions of the clones referenced in Table 2 are provided below in Table 3.

TABLE 3

Variable Region Sequences of anti-CD33 Antibodies with Conditional Affinity

| | | | |
|---|---|---|---|
| mAb ID | VL Seg Name | Heavy Chain Variable Region | Light Chain Variable Region |
| P01_ A09 | IGKV 1-39-SL | QVQLVESGGGLVQAGGSLRLSCA ASRRSSRSWAMHWVRQAPGKGL EWVAVISYDGRLKYYADSVKGRFT ISRDNAEYLVYLQMNSLRAEDTAV YYCAAERPTSDGSTGGMDV WGQ GTLVTVSS (SEQ ID NO: 3) | DIQMTQSPSSLSASVGDRVTITCR ASQSISSYLN WYQQKPGKAPKLL IYGASTLQS GVPSRFSGSGSGTD FTLTISSLQPEDFATYYCQQSYSS GT FGQGTKVEIK (SEQ ID NO: 4) |
| P01_ C05 | IGKV 1-39-SL | QVQLVESGGGLVQAGGSLRLSCA ASRRSSRSWAMHWVRQAPGKGL EWVAVISYDGRLKYYADSVKGRFT ISRDNAEYLVYLQMNSLRAEDTAV YYCAAEQWGTNDGPDGGMDY W GQGTLVTVSS (SEQ ID NO: 5) | DIQMTQSPSSLSASVGDRVTITCR ASQTIGSHLN WYQQKPGKAPKLL IYGTSNLQS GVPSRFSGSGSGTD FTLTISSLQPEDFATYYCQQSYSS PLT FGQGTKVEIK (SEQ ID NO: 6) |
| P01_ F05 | IGKV 1-39-SL | QVQLVESGGGLVQAGGSLRLSCA ASRRSSRSWAMHWVRQAPGKGL EWVAVISYDGRLKYYADSVKGRFT ISRDNAEYLVYLQMNSLRAEDTAV YYCAAGPDRWAGYDAFDV WGQG TLVTVSS (SEQ ID NO: 7) | DIQMTQSPSSLSASVGDRVTITCR ASQTINTHLN WYQQKPGKAPKLL IYGASNLQG GVPSRFSGSGSGT DFTLTISSLQPEDFATYYCQQSYS TPLT FGQGTKVEIK (SEQ ID NO: 8) |
| P02_ A10 | IGKV 1-39-SL | QVQLVESGGGLVQAGGSLRLSCA ASRRSSRSWAMHWVRQAPGKGL EWVAVISYDGRLKYYADSVKGRFT ISRDNAEYLVYLQMNSLRAEDTAV YYCAAESEWDTGGFDV WGQGTL VTVSS (SEQ ID NO: 9) | DIQMTQSPSSLSASVGDRVTITCR ASQSIGSYLN WYQQKPGKAPKLL IYSSSNLQS GVPSRFSGSGSGTD FTLTISSLQPEDFATYYCQQSYTS GT FGQGTKVEIK (SEQ ID NO: 10) |
| P02_ C02 | IGKV 1-39-SL | QVQLVESGGGLVQAGGSLRLSCA ASRRSSRSWAMHWVRQAPGKGL EWVAVISYDGRLKYYADSVKGRFT ISRDNAEYLVYLQMNSLRAEDTAV YYCAAEGGMGLDP WGQGTLVTV SS (SEQ ID NO: 11) | DIQMTQSPSSLSASVGDRVTITCR ASQTISSYLN WYQQKPGKAPKLLI YAASNLHS GVPSRFSGSGSGTD FTLTISSLQPEDFATYYCQQSYSS GT FGQGTKVEIK (SEQ ID NO: 12) |
| P02_ D08 | IGKV 1-39-SL | QVQLVESGGGLVQAGGSLRLSCA ASRRSSRSWAMHWVRQAPGKGL EWVAVISYDGRLKYYADSVKGRFT ISRDNAEYLVYLQMNSLRAEDTAV YYCAAERGDGLDGNVGLD IWGQ GTLVTVSS (SEQ ID NO: 13) | DIQMTQSPSSLSASVGDRVTITCR ASQSISKYLN WYQQKPGKAPKLL IYGASTLQS GVPSRFSGSGSGTD FTLTISSLQPEDFATYYCQQSYSS GT FGQGTKVEIK (SEQ ID NO: 14) |
| P02_ E11 | IGKV 1-39-SL | QVQLVESGGGLVQAGGSLRLSCA ASRRSSRSWAMHWVRQAPGKGL EWVAVISYDGRLKYYADSVKGRFT ISRDNAEYLVYLQMNSLRAEDTAV YYCAAEASTDNGLDY WGQGTLVT VSS (SEQ ID NO: 15) | DIQMTQSPSSLSASVGDRVTITCR ASQTISRHLN WYQQKPGKAPKLL IYSASSLAS GVPSRFSGSGSGTD FTLTISSLQPEDFATYYCQQSHSA GT FGQGTKVEIK (SEQ ID NO: 16) |
| P02_ H01 | IGKV 1-39-SL | QVQLVESGGGLVQAGGSLRLSCA ASRRSSRSWAMHWVRQAPGKGL EWVAVISYDGRLKYYADSVKGRFT ISRDNAEYLVYLQMNSLRAEDTAV YYCAAGSGGGDGLDV WGQGTLV TVSS (SEQ ID NO: 17) | DIQMTQSPSSLSASVGDRVTITCR ASQTISSYLN WYQQKPGKAPKLLI YSSSTLQG GVPSRFSGSGSGTD FTLTISSLQPEDFATYYCQQSYSS GT FGQGTKVEIK (SEQ ID NO: 18) |
| P03_ A01 | IGKV 1-39-SL | QVQLVESGGGLVQAGGSLRLSCA ASRRSSRSWAMHWVRQAPGKGL EWVAVISYDGRLKYYADSVKGRFT ISRDNAEYLVYLQMNSLRAEDTAV YYCAAETGSVGLDY WGQGTLVTV SS (SEQ ID NO: 19) | DIQMTQSPSSLSASVGDRVTITCR ASQGISTYLN WYQQKPGKAPKLL IYGATSLES GVPSRFSGSGSGTD FTLTISSLQPEDFATYYCQQGYS QG TFGQGTKVEIK (SEQ ID NO: 20) |
| P03_ G12 | IGKV 1-39-SL | QVQLVESGGGLVQAGGSLRLSCA ASRRSSRSWAMHWVRQAPGKGL EWVAVISYDGRLKYYADSVKGRFT ISRDNAEYLVYLQMNSLRAEDTAV YYCAADPTEGWSYLDYWGQGTL VTVSS (SEQ ID NO: 21) | DIQMTQSPSSLSASVGDRVTITCR ASQGISSYLN WYQQKPGKAPKLL IYGASRLQS GVPSRFSGSGSGTD FTLTISSLQPEDFATYYCQQGYSS GT FGQGTKVEIK (SEQ ID NO: 22) |
| P04_ C09 | IGKV 1-39-SL | QVQLVESGGGLVQAGGSLRLSCA ASRRSSRSWAMHWVRQAPGKGL EWVAVISYDGRLKYYADSVKGRFT ISRDNAEYLVYLQMNSLRAEDTAV YYCAAEANESDSGGIDV WGQGTL VTVSS (SEQ ID NO: 23) | DIQMTQSPSSLSASVGDRVTITCR ASQTIGGYLN WYQQKPGKAPKLL IYAASSLQS GVPSRFSGSGSGTD FTLTISSLQPEDFATYYCQQGYSS GA FGQGTKVEIK (SEQ ID NO: 24) |
| P07_ C04 | IGKV 1-39-SL | QVQLVESGGGLVQAGGSLRLSCA ASRRSSRSWAMHWVRQAPGKGL EWVAVISYDGRLKYYADSVKGRFT ISRDNAEYLVYLQMNSLRAEDTAV YYCAAEPDGEGTNGGLDI WGQGT LVTVSS (SEQ ID NO: 25) | DIQMTQSPSSLSASVGDRVTITCR ASQNISRYLN WYQQKPGKAPKLL IYSASSLQT GVPSRFSGSGSGTD FTLTISSLQPEDFATYYCQQSYSS GA FGQGTKVEIK (SEQ ID NO: 26) |
| P08_ C08 | IGKV 1-39-SL | QVQLVESGGGLVQAGGSLRLSCA ASRRSSRSWAMHWVRQAPGKGL EWVAVISYDGRLKYYADSVKGRFT ISRDNAEYLVYLQMNSLRAEDTAV YYCAAEGSGSTDGSGAIDV WGQG TLVTVSS (SEQ ID NO: 27) | DIQMTQSPSSLSASVGDRVTITCR ASQSIGSYLN WYQQKPGKAPKLL IYAASSLQS GVPSRFSGSGSGTD FTLTISSLQPEDFATYYCQQSYSS GS FGQGTKVEIK (SEQ ID NO: 28) |
| P16_ E06 | IGKV 1-39-SL | QVQLVESGGGLVQAGGSLRLSCA ASRRSSRSWAMHWVRQAPGKGL EWVAVISYDGRLKYYADSVKGRFT ISRDNAEYLVYLQMNSLRAEDTAV YYCAAEGGDYPGTSDGGLDI WGQ GTLVTVSS (SEQ ID NO: 29) | DIQMTQSPSSLSASVGDRVTITCR ASQSISNYLN WYQQKPGKAPKLL IYGASSLQS GVPSRFSGSGSGTD FTLTISSLQPEDFATYYCQQSYSA GT FGQGTKVEIK (SEQ ID NO: 30) |
| P01__ B02 | IGKV 1-39-SL | QVQLVESGGGLVQAGGSLRLSCA ASRRSSRSWAMHWVRQAPGKGL EWVAVISYDGRLKYYADSVKGRFT ISRDNAEYLVYLQMNSLRAEDTAV YYCAAADWYSGGGDALDI WGQG TLVTVSS (SEQ ID NO: 31) | DIQMTQSPSSLSASVGDRVTITCR ASQTIYNYLN WYQQKPGKAPKLL IYGASNLQS GVPSRFSGSGSGTD FTLTISSLQPEDFATYYCQQTYSS PT FGQGTKVEIK (SEQ ID NO: 32) |
| P02_ B03 | IGLV 1-47-SL | QVQLVESGGGLVQAGGSLRLSCA ASRRSSRSWAMHWVRQAPGKGL EWVAVISYDGRLKYYADSVKGRFT ISRDNAEYLVYLQMNSLRAEDTAV YYCAADQDVGSVGNYYGNGMDV WGQGTLVTVSS (SEQ ID NO: 33) | QSVLTQPPSASGTPGQRVTISCS GSSSNIGSNYVN WYQQLPGTAP KLLIYRNNQRPS GVPDRFSGSKS GTSASLAISGLRSEDEADYYCAA WDDNPRWV FGTGTKLTVL (SEQ ID NO: 34) |
| P02_ D09 | IGLV 1-47-SL | QVQLVESGGGLVQAGGSLRLSCA ASRRSSRSWAMHWVRQAPGKGL EWVAVISYDGRLKYYADSVKGRFT ISRDNAEYLVYLQMNSLRAEDTAV YYCAAEEGDGGFFDY WGQGTLVT VSS (SEQ ID NO: 35) | QSVLTQPPSASGTPGQRVTISCS GSSSNIGSNYVN WYQQLPGTAP KLLIYRNNERPS GVPDRFSGSKS GTSASLAISGLRSEDEADYYCAA WDGSLSGRGV FGTGTKLTVL (SEQ ID NO: 36) |
| P02_ E08 | IGLV 1-47-SL | QVQLVESGGGLVQAGGSLRLSCA ASRRSSRSWAMHWVRQAPGKGL EWVAVISYDGRLKYYADSVKGRFT ISRDNAEYLVYLQMNSLRAEDTAV YYCAASDYDGGGLFDL WGQGTLV TVSS (SEQ ID NO: 37) | QSVLTQPPSASGTPGQRVTISCS GSNSNIGDNYVH WYQQLPGTAP KLLIYMNNQRPS GVPDRFSGSKS GTSASLAISGLRSEDEADYYCAT WDDSLSGVV FGTGTKLTVL (SEQ ID NO: 38) |
| P03_ H04 | IGLV 1-47-SL | QVQLVESGGGLVQAGGSLRLSCA ASRRSSRSWAMHWVRQAPGKGL EWVAVISYDGRLKYYADSVKGRFT ISRDNAEYLVYLQMNSLRAEDTAV YYCAADPHDGYGGGPGFDY WGQ GTLVTVSS (SEQ ID NO: 39) | QSVLTQPPSASGTPGQRVTISCS GSSSNIGSNYVY WYQQLPGTAPK LLIYRSNQRPS GVPDRFSGSKSG TSASLAISGLRSEDEADYYCAAW DDTLSAVV FGTGTKLTVL (SEQ ID NO: 40) |
| P04_ C05 | IGLV 1-47-SL | QVQLVESGGGLVQAGGSLRLSCA ASRRSSRSWAMHWVRQAPGKGL EWVAVISYDGRLKYYADSVKGRFT ISRDNAEYLVYLQMNSLRAEDTAV YYCAAGLPWTDY WGQGTLVTVSS (SEQ ID NO: 41) | QSVLTQPPSASGTPGQRVTISCS GSNSNIGSNYVS WYQQLPGTAP KLLIYSNNQRPS GVPDRFSGSKS GTSASLAISGLRSEDEADYYCAA WDDRLSVV FGTGTKLTVL (SEQ ID NO: 42) |

Also, CDRs and SEQ ID Nos of the CDRs of the conditionally specific anti-CD33 antibodies of Table 3 are provided below in Table 4.

TABLE 4

Anti-CD33 antibodies having conditional affinity and their antigen-binding CDR sequences according to Kabat (underlined) and Chothia (bold).

| | | | | |
|---|---|---|---|---|
| mAb ID | | CDR1 | CDR2 | CDR3 |
| P01_A09 | H | RRSSRSWAMH (SEQ ID Nos: 43 (whole), 116, and 117) | VISYDGRLKYYADSVKGR F (SEQ ID Nos: 44 (whole) and 118) | ERPTSDGSTGGMDV (SEQ ID NO: 45) |
| | L | RASQSISSYLN (SEQ ID NO: 46) | GASTLQS (SEQ ID NO: 47) | QQSYSSGT (SEQ ID NO: 48) |
| P01_C05 | H | RRSSRSWAMH (SEQ ID Nos: 43 (whole), 116, and 117) | VISYDGRLKYYADSVKGR F (SEQ ID Nos: 44 (whole) and 118) | EQWGTNDGPDGGMD Y (SEQ ID NO: 49) |
| | L | RASQTIGSHLN (SEQ ID NO: 50) | GTSNLQS (SEQ ID NO: 51) | QQSYSSPLT (SEQ ID NO: 52) |
| P01_F05 | H | RRSSRSWAMH (SEQ ID Nos: 43 (whole), 116, and 117) | VISYDGRLKYYADSVKGR F (SEQ ID Nos: 44 (whole) and 118) | GPDRWAGYDAFD V (SEQ ID NO: 53) |
| | L | RASQTINTHLN (SEQ ID NO: 54) | GASNLQG (SEQ ID NO: 55) | QQSYSTPLT (SEQ ID NO: 56) |
| P02_A10 | H | RRSSRSWAMH (SEQ ID Nos: 43 (whole), 116, and 117) | VISYDGRLKYYADSVKGR F (SEQ ID Nos: 44 (whole) and 118) | ESEWDTGGFDV (SEQ ID NO: 57) |
| | L | RASQSIGSYLN (SEQ ID NO: 58) | SSSNLQS (SEQ ID NO: 59) | QQSYTSGT (SEQ ID NO: 60) |
| P02_C02 | H | RRSSRSWAMH (SEQ ID Nos: 43 (whole), 116, and 117) | VISYDGRLKYYADSVKGR F (SEQ ID Nos: 44 (whole) and 118) | EGGMGLDP (SEQ ID NO: 61) |
| | L | RASQTISSYLN (SEQ ID NO: 62) | AASNLHS (SEQ ID NO: 63) | QQSYSSGT (SEQ ID NO: 48) |
| P02_D08 | H | RRSSRSWAMH (SEQ ID Nos: 43 (whole), 116, and 117) | VISYDGRLKYYADSVKGR F (SEQ ID Nos: 44 (whole) and 118) | ERGDGLDGNVGLDI (SEQ ID NO: 64) |
| | L | RASQSISKYLN (SEQ ID NO: 65) | GASTLQS (SEQ ID NO: 47) | QQSYSSGT (SEQ ID NO: 48) |
| P02_E11 | H | RRSSRSWAMH (SEQ ID Nos: 43 (whole), 116, and 117) | VISYDGRLKYYADSVKGR F (SEQ ID Nos: 44 (whole) and 118) | EASTDNGLDY (SEQ ID NO: 66) |
| | L | RASQTISRHLN (SEQ ID NO: 67) | SASSLAS (SEQ ID NO: 68) | QQSHSAGT (SEQ ID NO: 69) |
| P02_H01 | H | RRSSRSWAMH (SEQ ID Nos: 43 (whole), 116, and 117) | VISYDGRLKYYADSVKGR F (SEQ ID Nos: 44 (whole) and 118) | GSGGGDGLDV (SEQ ID NO: 70) |
| | L | RASQTISSYLN (SEQ ID NO: 62) | SSSTLQG (SEQ ID NO: 71) | QQSYSSGT (SEQ ID NO: 48) |
| P03_A01 | H | RRSSRSWAMH (SEQ ID Nos: 43 (whole), 116, and 117) | VISYDGRLKYYADSVKGR F (SEQ ID Nos: 44 (whole) and 118) | ETGSVGLDY (SEQ ID NO: 72) |
| | L | RASQGISTYLN (SEQ ID NO: 73) | GATSLES (SEQ ID NO: 74) | QQGYSQGT (SEQ ID NO: 75) |
| P03_G12 | H | RRSSRSWAMH (SEQ ID Nos: 43 (whole), 116, and 117) | VISYDGRLKYYADSVKGR F (SEQ ID Nos: 44 (whole) and 118) | DPTEGWSYLDY (SEQ ID NO: 76) |
| | L | RASQGISSYLN (SEQ ID NO: 77) | GASRLQS (SEQ ID NO: 78) | QQGYSSGT (SEQ ID NO: 79) |
| P04_C09 | H | RRSSRSWAMH (SEQ ID Nos: 43 (whole), 116, and 117) | VISYDGRLKYYADSVKGR F (SEQ ID Nos: 44 (whole) and 118) | EANESDSGGIDV (SEQ ID NO: 80) |
| | L | RASQTIGGYLN (SEQ ID NO: 81) | AASSLQS (SEQ ID NO: 82) | QQGYSSGA (SEQ ID NO: 83) |
| P07_C04 | H | RRSSRSWAMH (SEQ ID Nos: 43 (whole), 116, and 117) | VISYDGRLKYYADSVKGR F (SEQ ID Nos: 44 (whole) and 118) | EPDGEGTNGGLDI (SEQ ID NO: 84) |
| | L | RASQNISRYLN (SEQ ID NO: 85) | SASSLQT (SEQ ID NO: 86) | QQSYSSGA (SEQ ID NO: 87) |
| P08_C08 | H | RRSSRSWAMH (SEQ ID Nos: 43 (whole), 116, and 117) | VISYDGRLKYYADSVKGR F (SEQ ID Nos: 44 (whole) and 118) | EGSGSTDGSGAIDV (SEQ ID NO: 88) |
| | L | RASQSIGSYLN (SEQ ID NO: 58) | AASSLQS (SEQ ID NO: 82) | QQSYSSGS (SEQ ID NO: 89) |
| P16_E06 | H | RRSSRSWAMH (SEQ ID Nos: 43 (whole), 116, and 117) | VISYDGRLKYYADSVKGR F (SEQ ID Nos: 44 (whole) and 118) | EGGDYPGTSDGGLDI (SEQ ID NO: 90) |
| | L | RASQSISNYLN (SEQ ID NO: 91) | GASSLQS (SEQ ID NO: 92) | QQSYSAGT (SEQ ID NO: 93) |
| P01_B02 | H | RRSSRSWAMH (SEQ ID Nos: 43 (whole), 116, and 117) | VISYDGRLKYYADSVKGR F (SEQ ID Nos: 44 (whole) and 118) | ADWYSGGGDALDI (SEQ ID NO: 94) |
| | L | RASQTIYNYLN (SEQ ID NO: 95) | GASNLQS (SEQ ID NO: 164) | QQTYSSPT (SEQ ID NO: 97) |
| P02_B03 | H | RRSSRSWAMH (SEQ ID Nos: 43 (whole), 116, and 117) | VISYDGRLKYYADSVKGR F (SEQ ID Nos: 44 (whole) and 118) | DQDVGSVGNYYGNGM DV (SEQ ID NO: 98) |
| | L | SGSSSNIGSNYVN (SEQ ID NO: 99) | RNNQRPS (SEQ ID NO: 96) | AAWDDNPRWV (SEQ ID NO: 100) |
| P02_D09 | H | RRSSRSWAMH (SEQ ID Nos: 43 (whole), 116, and 117) | VISYDGRLKYYADSVKGR F (SEQ ID Nos: 44 (whole) and 118) | EEGDGGFFDY (SEQ ID NO: 101) |
| | L | SGSSSNIGSNYVN (SEQ ID NO: 99) | RNNERPS (SEQ ID NO: 102) | AAWDGSLSGRGV (SEQ ID NO: 103) |
| P02_E08 | H | RRSSRSWAMH (SEQ ID Nos: 43 (whole), 116, and 117) | VISYDGRLKYYADSVKGR F (SEQ ID Nos: 44 (whole) and 118) | SDYDGGGLFDL (SEQ ID NO: 104) |
| | L | SGSNSNIGDNYVH (SEQ ID NO: 105) | MNNQRPS (SEQ ID NO: 106) | ATWDDSLSGW (SEQ ID NO: 107) |
| P03_H04 | H | RRSSRSWAMH (SEQ ID Nos: 43 (whole), 116, and 117) | VISYDGRLKYYADSVKGR F (SEQ ID Nos: 44 (whole) and 118) | DPHDGYGGGPGF DY (SEQ ID NO: 108) |
| | L | SGSSSNIGSNYVY (SEQ ID NO: 109) | RSNQRPS (SEQ ID NO: 110) | AAWDDTLSAVV (SEQ ID NO: 111) |
| P04_C05 | H | RRSSRSWAMH (SEQ ID Nos: 43 (whole), 116, and 117) | VISYDGRLKYYADSVKGR F (SEQ ID Nos: 44 (whole) and 118) | GLPWTDY (SEQ ID NO: 112) |
| | L | SGSNSNIGSNYVS (SEQ ID NO: 113) | SNNQRPS (SEQ ID NO: 114) | AAWDDRLSVV (SEQ ID NO: 115) |

As shown in the data provided above, multiple different anti-CD33 clones having conditional affinity were isolated. Most of these anti-CD33 clones have reduced affinity for CD33 in the presence of 10 µM MTX, as compared to in the presence of 0 µM MTX. In addition, at least one anti-CD33 clone was identified that has increased affinity for CD33 in the presence of 10 µM MTX, as compared to in the presence of 0 µM MTX.

### Example 3: Selection of Anti-EGFR Antibodies Having Conditional Affinity

This Example describes selection of anti-EGFR antibodies having conditional affinity.

Antibodies toward human epidermal growth factor receptor (“EGFR”) (UniProt Accession Number: P00533) that exhibit diminished binding affinities in the presence methotrexate (“MTX”) were identified from the phage displayed libraries described in Example 1 by subjecting the libraries to 5 rounds of panning. The first 4 rounds of panning used biotinylated EGFR antigen captured on streptavidin or neutravidin coated 96-well plates. The 5^{th} round of panning was performed in solution by preincubating the phage displayed antibodies with biotinylated antigen then capturing them on a neutravidin coated 96-well plate. 1536 clones were randomly chosen from the 4^{th} and 5^{th} round of panning against the EGFR antigen and their binding specificity determined by phage ELISA. Clones that specifically bound to EGFR were sequenced and all unique clones were subjected to a phage competition ELISA to assess their relative affinities to EGFR and sensitivity to MTX by measuring their binding strengths in the presence or absence of 10 µM (micromolar) MTX (i.e. in 0 or 10 µM MTX). Clones that demonstrated a decrease in binding in the presence of MTX were expressed as scFv-Fc fusions and their EGFR binding kinetics in the presence and absence of 10 µM MTX were measured using a biosensor as described in Example 2.

Table 5 provides kinetic data for various anti-EGFR clones having conditional affinity for EGFR identified through this screening process. As shown in Table 5, numerous clones were selected for which have an antibody-antigen K_{D} in 10 µM MTX / antibody-antigen K_{D} in 0 µM MTX ratio of greater than, for example, 2, 3, 4, 5, or 10. The data in Table 5 may be interpreted as described above in Example 2.

Of note, the majority of the anti-EGFR clones analyzed have less than 2-fold difference in binding affinity at 0 µM MTX vs. 10 µM MTX; most of these clones are not included in Table 5. However, for reference purposes, Table 5 also includes kinetic data for 2 of these types of anti-EGFR clones (P01_B06 and P08_E05).

TABLE 5

| | Kinetics toward antigen with 0 µM MTX | | | Kinetics toward antigen with 10 µM MTX | | | |
|---|---|---|---|---|---|---|---|
| Clone | kon (1/Ms) | koff (1/s) | K_{D} (nM) | kon (1/Ms) | koff (1/s) | K_{D} (nM)* | Ratio: K_{D} with MTX / K_{D} without MTX |
| P14_E03 | 1.1E+05 | 6.7E-04 | 6.3 | 2.9E+04 | 7.2E-04 | 25 | 3.9 |
| P15_F02 | 8.2E+04 | 2.2E-02 | 266 | 5.0E+04 | 1.3E-01 | 2530 | 9.5 |
| P08_D06 | 7.1E+04 | 1.3E-02 | 185 | 2.4E+04 | 3.3E-02 | 1393 | 7.5 |
| P01_H04 | 8.5E+04 | 7.6E-02 | 889 | - | - | >1800 | >2.0 |
| P07_H08 | 3.3E+05 | 2.7E-01 | 823 | - | - | >9371 | >11 |
| P05_A11 | 1.2E+05 | 2.8E-02 | 235 | 8.7E+04 | 5.0E-02 | 580 | 2.5 |
| P08_E05 | 2.0E+05 | 7.1E-02 | 360 | 1.5E+05 | 7.3E-02 | 480 | 1.3 |
| P01_F02 | 6.1E+04 | 1.1E-02 | 188 | - | - | >9409 | >50 |
| P01_B06 | 1.0E+05 | 2.4E-02 | 230 | 1.0E+05 | 2.6E-02 | 256 | 1.1 |

“*”: For interactions where kₒₙ and k_{off} are reported, the K_{D} was calculated using k_{off}/kₒₙ. For weak interactions in which kinetic rate constants could not be accurately determined (e.g. some of the cases of antigen-antibody interaction in the presence of 10 µM MTX), estimates of the K_{D} were made from the binding response at the highest antigen concentration tested, or based on the typical limit of detection for a Biacore T200.

The amino acid sequences of the VH and VL regions of the clones described in Table 5 are provided below in Table 6.

TABLE 6

Variable Region Sequences of anti-EGFR Antibodies with Conditional Affinity

| | | | |
|---|---|---|---|
| mAb ID | VL Seg Name | Heavy Chain Variable Region | Light Chain Variable Region |
| P01_F02 | IGLV1 -47-SL | QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAADVESALAYGEPYHHMDV WGQGTLVTVSS (SEQ ID NO: 119) | QSVLTQPPSASGTPGQRVTISCSGSSSNIGRNYVY WYQQLPGTAPKLLIYRTSQRPS GVPDRFSGSKSGTSASLAISGLRSEDEADYYCATWDDSLSGWV FGTGTKLTVL (SEQ ID NO: 120) |
| P07_H08 | IGKV1 -39-SL | QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAAGFDWGWDSSDIYSAMDY WGQGTLVTVSS (SEQ ID NO: 121) | DIQMTQSPSSLSASVGDRVTITCRASQNIGRYLH WYQQKPGKAPKLLIYSASNLQS GVPSRFSGSGSGTDFTLTISSLQPEDFATYYCQQSYSSPFTF GQGTKVEIK(SEQ ID NO: 122) |
| P08_D06 | IGLV1 -47-SL | QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAADDDGDVYAGYSYGGFDY WGQGTLVTVSS (SEQ ID NO: 123) | QSVLTQPPSASGTPGQRVTISCSGSSSNIGRNYVY WYQQLPGTAPKLLIYRNDQRPS GVPDRFSGSKSGTSASLAISGLRSEDEADYYCAAWDDSLSGWV FGTGTKLTVL (SEQ ID NO: 124) |
| P14_E03 | IGLV1 -47-SL | QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAAAPRVDWSDVSSGIDY WGQGTLVTVSS (SEQ ID NO: 125) | QSVLTQPPSASGTPGQRVTISCSGSISNIGTNNVY WYQQLPGTAPKLLIYRNNQRPS GVPDRFSGSKSGTSASLAISGLRSEDEADYYCSAWDDSLSGVV FGTGTKLTVL (SEQ ID NO: 126) |
| P01_H04 | IGLV1 -47-SL | QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAADMDDMLSYYNSFDI WGQGTLVTVSS (SEQ ID NO: 127) | QSVLTQPPSASGTPGQRVTISCSGSSSNIGRNYVY WYQQLPGTAPKLLIYRNNQRPS GVPDRFSGSKSGTSASLAISGLRSEDEADYYCAAWDDSLSGWV FGTGTKLTVL (SEQ ID NO: 128) |
| P01_B06 | IGLV1 -47-SL | QVQLVESGGGLVQAGGSLRLSCAASRRSSRSWAMHWVRQAPGKGLEWVAVISYDGRLKYYADSVKGRFTISRDNAEYLVYLQMNSLRAEDTAVYYCAADTSMFASYDPMDV WGQG TLVTVSS (SEQ ID NO: 129) | QSVLTQPPSASGTPGQRVTISCSGSSSNIGRNYVN WYQQLPGTAPKLLIYRDDHRPS GVPDRFSGSKSGTSASLAISGLRSEDEADYYCASWDDTLSGWV FGTGTKLTVL (SEQ ID NO: 130) |
| P08_E05 | IGLV1 -47-SL | QVQLVESGGGLVQAGGSLRLSCA ASRRSSRSWAMHWVRQAPGKGL EWVAVISYDGRLKYYADSVKGRFT ISRDNAEYLVYLQMNSLRAEDTAV YYCAADGKGAAWSEVDEPDV WG QGTLVTVSS (SEQ ID NO: 131) | QSVLTQPPSASGTPGQRVTISC SGSSSNIGSNYVY WYQQLPGT APKLLIYQNNQRPS GVPDRFSG SKSGTSASLAISGLRSEDEADY YCAAWDDSLSGLV FGTGTKLT VL (SEQ ID NO: 132) |
| P05_A11 | IGLV1 -47-SL | QVQLVESGGGLVQAGGSLRLSCA ASRRSSRSWAMHWVRQAPGKGL EWVAVISYDGRLKYYADSVKGRFT ISRDNAEYLVYLQMNSLRAEDTAV YYCAASDDADIFDDYSYGAMDY W GQGTLVTVSS (SEQ ID NO: 133) | QSVLTQPPSASGTPGQRVTISC SGSSSNIGRNYVY WYQQLPGT APKLLIYMTSQRPS GVPDRFSG SKSGTSASLAISGLRSEDEADY YCASWDDNLRAWV FGTGTKLT VL (SEQ ID NO: 134) |
| P15_F02 | IGLV1 -47-SL | QVQLVESGGGLVQAGGSLRLSCA ASRRSSRSWAMHWVRQAPGKGL EWVAVISYDGRLKYYADSVKGRFT ISRDNAEYLVYLQMNSLRAEDTAV YYCAAARDVDGSGYDTIDV WGQG TLVTVSS (SEQ ID NO: 135) | QSVLTQPPSASGTPGQRVTISC SGSSSNIGRNYVY WYQQLPGT APKLLIYSNNQRPS GVPDRFSG SKSGTSASLAISGLRSEDEADY YCAAWDDSLSGVWV FGTGTKL TVL (SEQ ID NO: 136) |

Also, CDRs and SEQ ID Nos of the CDRs of the anti-EGFR antibodies having conditional affinity of Table 6 are provided below in Table 7.

TABLE 7

Anti-EGFR antibodies having conditional affinity and their antigen-binding CDR sequences according to Kabat (underlined) and Chothia (bold).

| | | | | |
|---|---|---|---|---|
| mAb ID | | CDR1 | CDR2 | CDR3 |
| P01_F02 | H | RRSSRSWAMH (SEQ ID Nos: 43 (whole), 116, and 117) | VISYDGRLKYYADSVKGRF (SEQ ID Nos: 44 (whole) and 118) | DVESALAYGEPYHHMDV (SEQ ID NO: 137) |
| | L | SGSSSNIGRNYVY (SEQ ID NO: 138) | RTSQRPS (SEQ ID NO: 139) | ATWDDSLSGWV (SEQ ID NO: 140) |
| P07_H08 | H | RRSSRSWAMH (SEQ ID Nos: 43 (whole), 116, and 117) | VISYDGRLKYYADSVKGRF (SEQ ID Nos: 44 (whole) and 118) | GFDWGWDSSDIYSAMDY (SEQ ID NO: 141) |
| | L | RASQNIGRYLH (SEQ ID NO: 142) | SASNLQS (SEQ ID NO: 143) | QQSYSSPFT (SEQ ID NO: 144) |
| P08_D06 | H | RRSSRSWAMH (SEQ ID Nos: 43 (whole), 116, and 117) | VISYDGRLKYYADSVKGRF (SEQ ID Nos: 44 (whole) and 118) | DDDGDVYAGYSYGGFDY (SEQ ID NO: 145) |
| | L | SGSSSNIGRNYVY (SEQ ID NO: 138) | RNDQRPS (SEQ ID NO: 146) | AAWDDSLSGWV (SEQ ID NO: 147) |
| P14_E03 | H | RRSSRSWAMH (SEQ ID Nos: 43 (whole), 116, and 117) | VISYDGRLKYYADSVKGRF (SEQ ID Nos: 44 (whole) and 118) | APRVDWSDVSSGIDY (SEQ ID NO: 148) |
| | L | SGSISNIGTNNVY (SEQ ID NO: 149) | RNNQRPS (SEQ ID NO: 96) | SAWDDSLSGVV (SEQ ID NO: 150) |
| P01_H04 | H | RRSSRSWAMH (SEQ ID Nos: 43 (whole), 116, and 117) | VISYDGRLKYYADSVKGRF (SEQ ID Nos: 44 (whole) and 118) | DMDDMLSYYNSFDI (SEQ ID NO: 151) |
| | L | SGSSSNIGRNYVY (SEQ ID NO: 138) | RNNQRPS (SEQ ID NO: 96) | AAWDDSLSGWV (SEQ ID NO: 147) |
| P01_B06 | H | RRSSRSWAMH (SEQ ID Nos: 43 (whole), 116, and 117) | VISYDGRLKYYADSVKGRF (SEQ ID Nos: 44 (whole) and 118) | DTSMFASYDPMDV (SEQ ID NO: 152) |
| | L | SGSSSNIGRNYVN (SEQ ID NO: 153) | RDDHRPS (SEQ ID NO: 154) | ASWDDTLSGWV (SEQ ID NO: 155) |
| P08_E05 | H | RRSSRSWAMH (SEQ ID Nos: 43 (whole), 116, and 117) | VISYDGRLKYYADSVKGRF (SEQ ID Nos: 44 (whole) and 118) | DGKGAAWSEVDEPDV (SEQ ID NO: 156) |
| | L | SGSSSNIGSNYVY (SEQ ID NO: 109) | QNNQRPS (SEQ ID NO: 157) | AAWDDSLSGLV (SEQ ID NO: 158) |
| P05_A11 | H | RRSSRSWAMH (SEQ ID Nos: 43 (whole), 116, and 117) | VISYDGRLKYYADSVKGRF (SEQ ID Nos: 44 (whole) and 118) | SDDADIFDDYSYGAMDY (SEQ ID NO: 159) |
| | L | SGSSSNIGRNYVY (SEQ ID NO: 138) | MTSQRPS (SEQ ID NO: 160) | ASWDDNLRAWV (SEQ ID NO: 161) |
| P15_F02 | H | RRSSRSWAMH (SEQ ID Nos: 43 (whole), 116, and 117) | VISYDGRLKYYADSVKGRF (SEQ ID Nos: 44 (whole) and 118) | ARDVDGSGYDTIDV (SEQ ID NO: 162) |
| | L | SGSSSNIGRNYVY (SEQ ID NO: 138) | SNNQRPS (SEQ ID NO: 114) | AAWDDSLSGVWV (SEQ ID NO: 163) |

As shown in the data provided above, multiple different anti-EGFR clones having conditional affinity were isolated. Most of these anti-EGFR clones have reduced affinity for EGFR in the presence of 10 µM MTX, as compared to in the presence of 0 µM MTX.

### Example 4: Specific in Vitro Cytotoxicity of CD33-Specific Conditional CAR T Cells and Short-Term Inhibition of their Activity

This example examines the target specificity and cytotoxicity of CAR T cells that contain an antibody with conditional affinity as described herein, in which the antibody is in scFv format in the chimeric antigen receptor (CAR) of the CAR T cells. CAR T cells that contain an antibody with conditional affinity as described herein in the scFv of the CAR may also be referred to as “conditional CAR T cells”, “conditional CAR Ts”, or the like.

In this example, the specific activity of different conditional CAR T cells was examined, each of which contained a different clone of an anti-CD33 antibody having conditional affinity in the respective CAR. The anti-CD33 clones tested were: P02_E11, P03_A01, P07_C04P02_E08, and P03_H04, which are described previously in, for example, Tables 2, 3, and 4. In addition, a positive control CAR T cell was prepared which contained a humanized IgG1 anti-CD33 antibody (i.e. which is not an antibody having conditional affinity) in the scFv of the CAR; this positive control CAR T cell is referred to herein as a “tool” CAR T cell. Untransduced T cells were also prepared. Also, each of the different conditional CAR T cells described above was generated in duplicate from PBMC cells from two different human donors.

Two target cell lines were used to evaluate the specific activity of the various different conditional CAR T cells: 1) a CD33-expressing, human acute myeloid leukemia cell line, “Molm-13” [obtained from DSMZ (Braunschweig, Germany)], and 2) a CD33-negative human chronic myeloid leukemia cell line, “K-562” [obtained from American Type Culture Collection (Manassas, VA)]. Both cell lines were transduced to stably express GFP and firefly luciferase. Sorted cells (GFP-positive) were cultured in RPMI 1640 medium supplemented with 10% heat inactivated Hyclone fetal bovine serum (GE Healthcare Life Sciences).

In vitro cytotoxicity assays were carried out in 96-well plates by co-culturing 50,000 T cells with 100,000 target cells (Molm13 or K562), for each different T cell described above. Co-cultures were maintained in a final volume of 100 ul of RPMI 1640 medium for 48 hours at 37 C with 5% CO₂. Target cell killing was assessed upon addition of One-Glo Luciferase Assay System (Promega) by measuring the luminescence (2103 EnVision™ Multilabel Plate Reader).

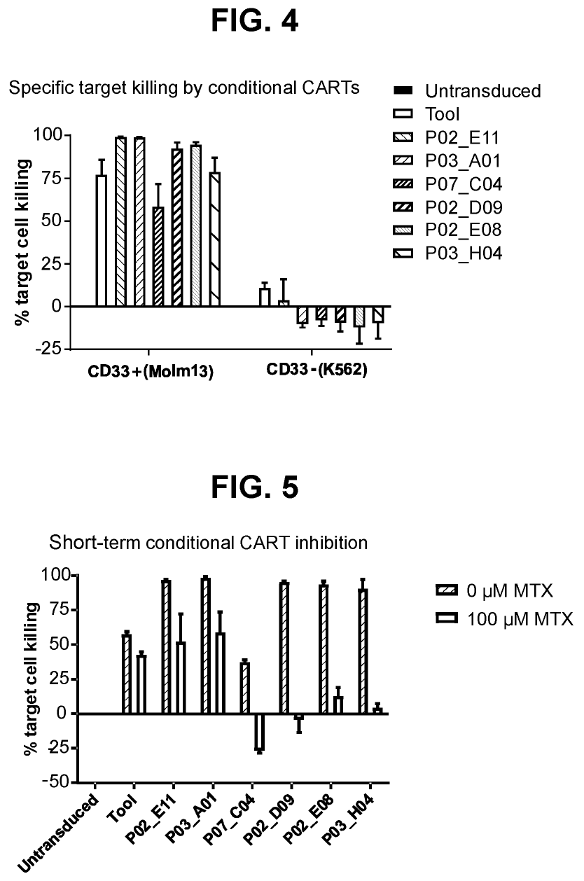

FIG. 4 shows the averaged results for each different CD33-specific CAR. In the graph, the Y-axis shows the % target cell killing, and the X-axis shows the target cell type [CD33 positive (Molm13) or CD33 negative (K562)] in the co-culture. The figure legend indicates the type of T cell in the co-culture. For additional reference, the type of T cell in the co-culture is listed here, in order from left to right along the X-axis: Molm13 co-cultures: [untransduced T cells (i.e. which do not contain a CAR)(these do not have a bar / value shown, as the value was 0%, per the “% target cell killing” calculation methodology described below); tool CAR T cell (i.e. positive control anti-CD33); P02_E11; P03_A01; P07_C04; P02_D09; P02_E08; and P03_H04]; K562 co-cultures: [untransduced T cells (these do not have a bar / value shown, as the value was 0%); tool T cell; P02_E11; P03_A01; P07_C04; P02_D09; P02_E08; and P03_H04]. “% target cell killing” was calculated by normalizing the luminescence values to those from co-cultures with untransduced T cells (i.e. which do not contain a CAR, and thus which are not expected to specifically target CD33-expressing cells). All of the tested anti-CD33 clones exhibited selective cytotoxicity against CD33-positive Molm13 cells.

This example demonstrates that CAR T cells which contain an anti-CD33 antibody having conditional affinity provided herein in the scFv of the CAR exhibit selective cytotoxicity against CD33-positive cells.

### Example 5: Short-Term Inhibition of CD33-Specific Conditional CAR T Cell Activity

This example examines the cytotoxicity of conditional CAR T cells containing various different anti-CD33 antibodies having conditional affinity against CD33-expressing target cells, in the presence and absence of methotrexate (MTX).

The anti-CD33 clones tested in this example were: P02_E11, P03_A01, P07_C04, P02_D09, P02_E08, and P03_H04. In addition, a tool CAR T cell was prepared as described in Example 4, as were untransduced T cells. Also, each of the different conditional CAR T cells described above was generated in duplicate from PBMC cells from two different human donors.

The target cell line used in this assay was MV4-11 (obtained from American Type Culture Collection), which expresses CD33. MV4-11 cells were transduced to stably express GFP and firefly luciferase, as described in Example 4.

In vitro cytotoxicity assays were carried out in 96-well plates by co-culturing 50,000 T cells with 100,000 MV4-11 cells, for each different T cells described above. In addition, each of the co-cultures contained either 100 µM MTX or 0 µM MTX. In both conditions, 1 mM leucovorin was supplemented to circumvent MTX toxicity. Co-cultures were maintained in a final volume of 100 µl of RPMI 1640 medium for 48 hours at 37 C with 5% CO₂. Target cell killing was assessed and plotted as described in Example 4.

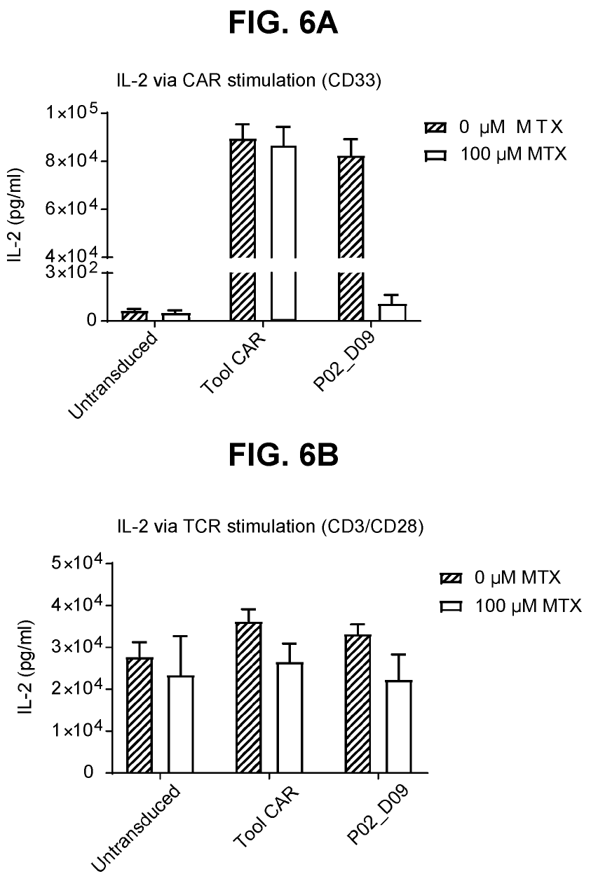

FIG. 5 shows the averaged results for each CD33-specific CAR, in the absence of MTX or the presence of 100 µM MTX. In the graph, the Y-axis shows the % target cell killing, and the X-axis shows the different CAR T cell in the co-culture. In addition, for each different T cell, two bars are present: left side: 0 µM MTX and right side: 100 µM MTX (also indicated in the figure legend). As shown in FIG. 5, all of the different clones of conditional CAR T cell exhibited significantly reduced cytotoxic activity towards the target cells in the presence of MTX as compared to in the absence of MTX (as indicated by the decrease in target cell killing in the presence of MTX as compared to in the absence of MTX). In contrast, conventional CAR T cells (Tool) were insensitive to the presence of MTX in terms of their target lysis efficiency. Without being bound by theory, it is believed that the MTX reduces the cytotoxic activity of the conditional CAR T cells by reducing the affinity of the anti-CD33 antibody in the CAR for the CD33 antigen in the target cell (and thereby reducing the binding of the CAR T cell to the target cell).

Thus, this experiment demonstrates that the cytotoxic activity of CAR T cells which contain an anti-CD33 antibody having conditional affinity provided herein in the scFv of the CAR can be modulated by MTX.

### Example 6: Affinity-Dependent Cytokine Secretion of CD33-Specific Conditional CAR T Cells

The example examines the levels secretion of the cytokines IL-2 and IFN-γ by conditional CAR T cells containing an anti-CD33 antibody having conditional affinity, after stimulation of the CAR T cell via either i) the CAR in the CAR T cell or ii) the TCR in the CAR T cell. In addition, these were examined in both the presence and absence of methotrexate (MTX).

The anti-CD33 clone used in this example was P02_D09. As shown in Table 2, the P02_D09 clone has significantly greater affinity toward CD33 (i.e. lower K_{D}) in the absence of MTX than in the presence of MTX (i.e. MTX reduces the affinity of P02_D09 for CD33). In addition, a tool T cell was prepared as described in Example 4, as were untransduced T cells.

The target cell line used in this assay was MV4-11, as described in Example 5.

Assays were carried out in 96-well plates by culturing 100,000 T cells with various other reagents (depending on the assay), for each of the different T cells described above. Each of the co-cultures contained either 100 µM MTX or 0 µM MTX. In both conditions, 1 mM leucovorin was supplemented to circumvent MTX toxicity. To stimulate the CAR T cells via the anti-CD33 CARs, 100,000 MV4-11 target cells (which express CD33) were included in the culture with the T cells. To stimulate the CAR T cells via the TCR, T cell Activation/Expansion Beads (Miltenyi) were included in the culture with the T cells (i.e. in the absence of target cells). T cell cultures (for TCR stimulation) or co-cultures (for CAR stimulation) were maintained in a final volume of 100 µl of RPMI 1640 medium for 24 hours at 37 C with 5% CO₂. The supernatant was then collected and frozen. The cytokines in the supernatant were measured using U-PLEX Th1/Th2 Combo plates (Meso-Scale Discovery; Rockville, MD) according to the manufacturer’s specifications.

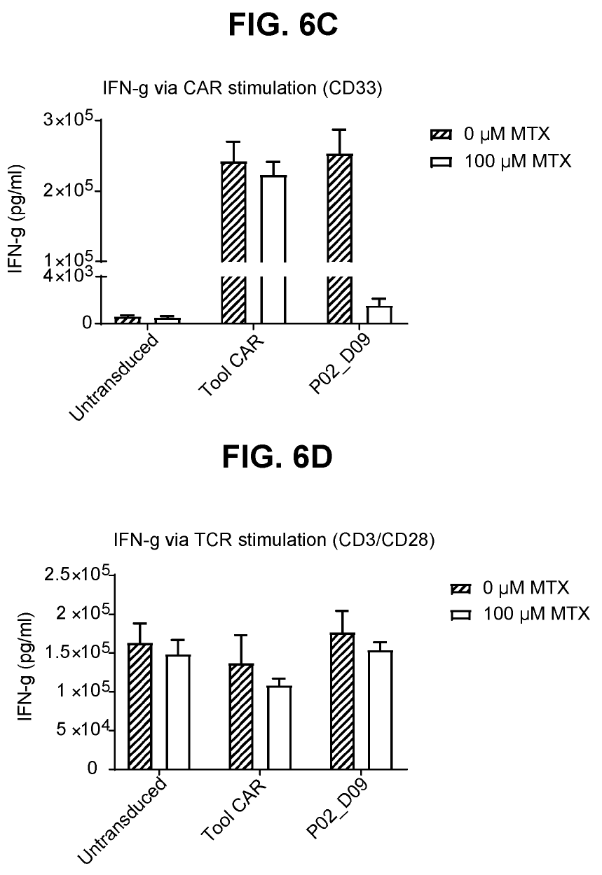

As shown in FIGS. 6A and 6C, upon activation of T cells via an anti-CD33 CAR (tool or conditional) IL-2 and IFN-γ secretion levels were similar between tool and conditional anti-CD33 CAR T cells in the absence of MTX. However, in the presence of MTX, cytokine production by P02_D09 was reduced back to the level comparable to that of unstimulated T cells (Untransduced) while the secretion remained high for the conventional CAR T cells.

As shown in FIGS. 6B and 6D, when T cells were activated in the presence of MTX through the stimulation of TCRs instead of CARs, the drastic decrease in cytokine release by the conditional CAR T cells seen with CAR-based stimulation was not observed. This provides additional evidence that the reduction in cytokine release in the presence of MTX by the CAR-stimulated cells containing an antibody having conditional affinity is not due to toxic effects of MTX but rather, it is due to reduced affinity of the CAR towards target protein.

This experiment thus provides additional evidence that the activation of a conditional CAR T cell containing an anti-CD33 antibody having conditional affinity provided herein in the scFv of the CAR can be modulated by MTX.

### Example 7: Affinity-Dependent Proliferation of CD33-Specific Conditional CAR T Cells

The example examines the proliferative capabilities of conditional CAR T cells containing an anti-CD33 antibody having conditional affinity after stimulation of the CAR T cell via either the CAR T cell’s i) CAR or ii) TCR. In addition, these were examined in both the presence and absence of methotrexate (MTX).

The anti-CD33 clone used in this example was P02_D09. In addition, a tool T cell was prepared as described in Example 4, as were untransduced T cells. T cells from one PBMC donor were used for this experiment.

The target cell line used in this assay was MV4-11, as described in Example 5.

The assays were carried out in 96-well plates by culturing 100,000 T cells with various other reagents (depending on the assay), for each of the different T cells described above. In addition, each of the co-cultures contained either 100 µM MTX or 0 µM MTX. In both conditions, 1 mM leucovorin was supplemented to circumvent MTX toxicity. The T cells were stimulated by CAR or TCR as in Example 6. Cultures were maintained in a final volume of 100 ul of RPMI 1640 medium for 7 days at 37 C with 5% CO₂. On day 7, cells were collected and counted via flow cytometry (BD LSR II) using 123Count eBeads (Thermo Fisher Scientific).

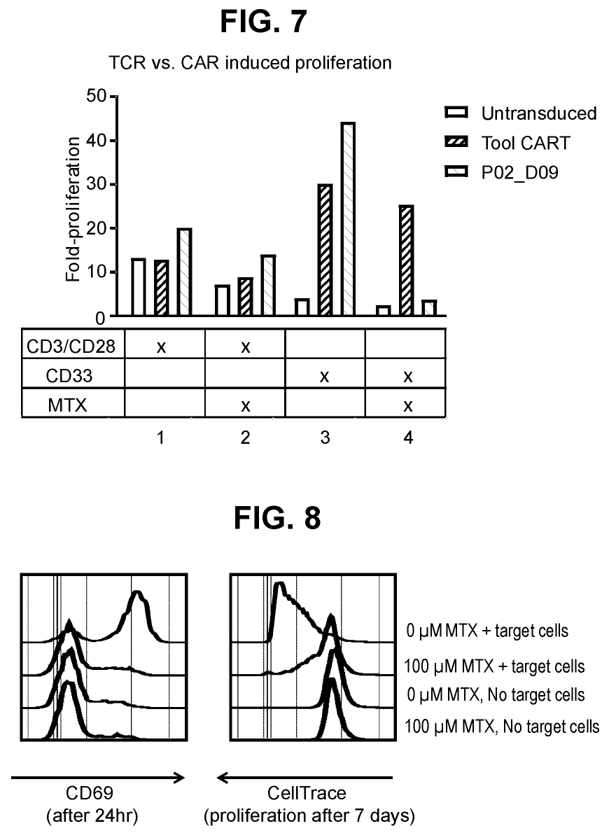

As shown in FIG. 7, the proliferative ability of CAR-stimulated P02_D09 CAR T cell is dramatically diminished in the presence of MTX, while conventional (tool) CAR T cells are not affected by MTX [compare the results depicted in column 3 (CAR stimulation; no MTX) and column 4 (CAR stimulation; 100 µM MTX)]. (In FIG. 7, “CD3/CD28” refers to TCR-stimulated cells, and “CD33” refers to CAR-stimualted cells.) For both P02_D09 and tool CAR T cells, proliferation of T cells is not affected by MTX when activated through the TCR [compare the results depicted in column 1 (TCR stimulation; no MTX) and column 2 (TCR stimulation; 100 µM MTX)], suggesting the inhibition of proliferation in the PO2-D09 CAR-stimulated T cell was due to MTX binding. Without being bound by theory, it is believed that the MTX reduces the proliferation of conditional CAR T cells by reducing the affinity of the anti-CD33 antibody in the CAR for the CD33 antigen in the target cell (thus reducing the binding of the CAR T cell to the CD33 antigen, and thereby reducing the subsequent activation and proliferation of CAR T cells that occurs upon the binding of a CAR T cell’s CAR to its antigen).

This experiment provides evidence that the proliferation of a conditional CAR T cell containing an anti-CD33 antibody having conditional affinity provided herein in the scFv of the CAR can be modulated by MTX.

### Example 8: Flow Cytometry Analysis of MTX-Dependent T Cell Activation and Proliferation

This example examines the effect of MTX on the activation and proliferation of conditional CAR T cells containing an anti-CD33 antibody having conditional affinity, as assessed by flow cytometry.

The anti-CD33 clone used in this example was P02_D09. CAR T cells containing this clone were generated in duplicate from PBMC cells from two different human donors. The target cell line used in this assay was MV4-11, as described in Example 5.

To assess activation of the conditional CAR T cells described above, in vitro cytotoxicity assays were carried out in 24-well plates by co-culturing 250,000 CAR T cells, with or without 250,000 MV4-11 cells. In addition, each of the co-cultures contained either 100 µM MTX or 0 µM MTX. In all conditions, 1 mM leucovorin was supplemented to circumvent MTX toxicity. Cells were collected after 24 hours of co-culture, labeled with fluorophore-conjugated antibody to CD69 (Biolegend) and analyzed by flow cytometry (BD LSR II). Activation was quantified via detection of the early activation marker, CD69.

To assess proliferative ability of the CAR T cells described above, the cells were prelabelled with the fluorescent intracellular dye, CellTrace Deep Red (Invitrogen), before co-culturing as described immediately above for the CAR T cell activation assay. The co-cultures were incubated for 7 days at 37 C with 5% CO₂. On day 7, cells were collected and analyzed via flow cytometry. Dilution of the CellTrace dye (leftward shift of peaks in the histogram) was interpreted as T cell proliferation.

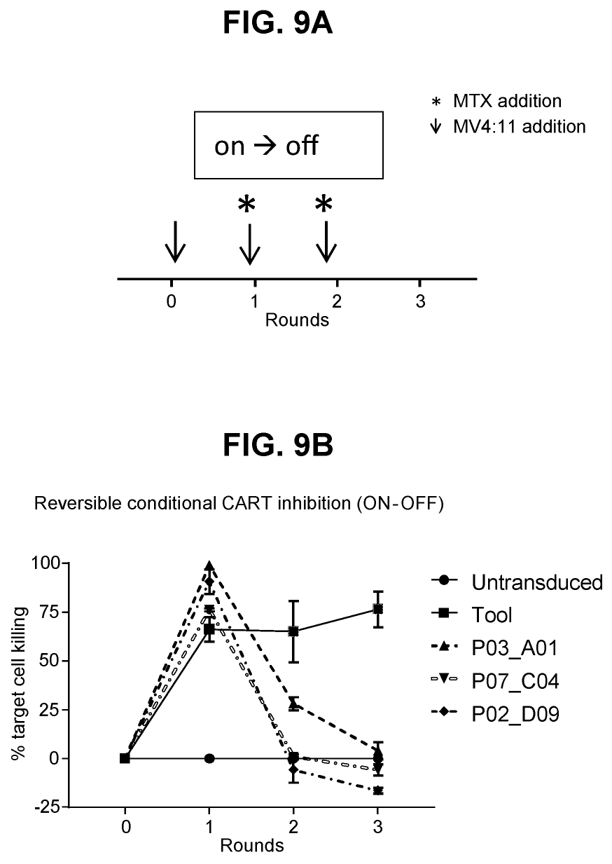

FIG. 8 shows that both activation as well as proliferation of P02_D09 is dramatically reduced in the presence of MTX. This can be seen by comparing the histograms of the 0 µM MTX + target cells co-cultures to the 100 µM MTX + target cells co-cultures for both the CD69 (activation) and CellTrace (proliferation) assays. In both assays, the 100 µM MTX + target cells co-culture histogram is distinctly different from the 0 µM MTX + target cell co-culture histogram, and it resembles the histogram for the cultures that contain no target cells. This indicates that the presence of 100 µM MTX reduces the activation and proliferation of conditional CAR T cells containing the anti-CD33 antibody P02_D09, and provides additional data indicating that CAR T cells containing an antibody having conditional affinity provided herein in the scFv of the CAR can be modulated by MTX.

### Example 9: Reversible Inhibition of CD33-Specific Conditional CAR T Cell Activity

This example demonstrates that CD33-specific conditional CAR T cell affinity toward CD33 can be reversible; active engagement of target cells by conditional CAR T cells can be halted while dormant T cells can be re-activated to lyse target cells by changing the concentration of MTX in the media.

The anti-CD33 clones tested in this example were: P03_A01, P07_C04, and P02_D09. In addition, a tool T cell was prepared as described in Example 4, as were untransduced T cells. Also, each of the different conditional CAR T cells described above was generated in triplicate from PBMC cells from three different human donors. The target cell line used in this assay was MV4-11; these cells were transduced to stably express GFP and firefly luciferase, as described in Example 5.

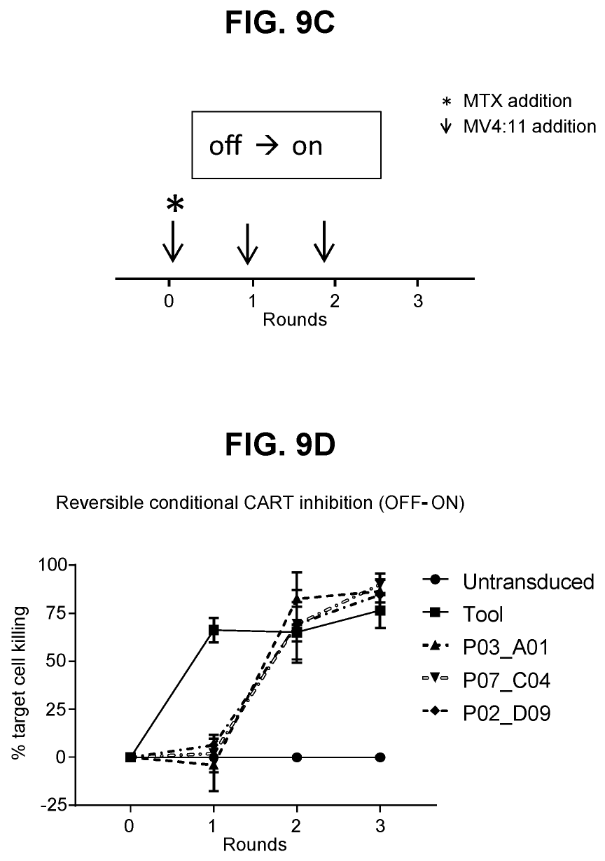

This experiment involved a series of in vitro cytotoxicity assays, in which co-cultures of T cells and MV4-11 target cells were incubated together for multiple rounds of sequential cytotoxicity assays, in which the MTX concentration changed at different rounds of the assays. In vitro cytotoxicity assays were carried out in 24-well plates by co-culturing 200,000 T cells with 200,000 MV4-11 cells, for each different T cells described above. In addition, each of the co-cultures contained either 100 µM MTX or 0 µM MTX at different times, as indicated by the schematics in FIGS. 9A and 9C. In both conditions, 1 mM leucovorin was supplemented to circumvent MTX toxicity. For each round of the assay, co-cultures were maintained in a final volume of 500 µl of RPMI 1640 medium at 37 C with 5% CO₂ for 48 hours. At the end of each round, cells were spun down (250 × g, 10 minutes), supernatant was removed, and fresh media containing fresh MV4-11 cells, leucovorin, and optionally MTX were added according to the schematics shown in FIGS. 9A and 9C. Target cell killing was assessed and plotted as described in Example 4.

FIGS. 9B and 9D show the averaged results for each CD33-specific CAR, for the different conditions as shown. Overall, all three of the conditional CAR T clones tested demonstrated reversible affinity toward CD33-expressing target cells, which could be modulated by the addition or removal of MTX.

In comparing the different panels of FIGS. 9A-9D, note that the schematics shown in FIGS. 9A and 9C indicate the MTX concentration in the co-culture at the start of the assay for a given round, whereas the results from that assay are shown the next round number in FIGS. 9B and 9D, respectively. So, for example, FIG. 9A indicates that there was no MTX at the start of the assays in Round 0; the results from these assays are plotted in FIG. 9B at Round 1. Then, after this round of the experiment was performed, fresh MV4-11 cells and other assay reagents were added to these same T cells (as described above), and MTX was also added to the co-culture (as shown in the FIG. 9A schematic for Round 1). The results from that round of co-culture were then plotted in FIG. 9B at Round 2, etc.

In the first scenario (testing the ability for an active CAR T cell containing an antibody having conditional affinity to be turned from “on” to “off”) (FIGS. 9A and 9B), conditional CAR T cells that are actively lysing the target cells (i.e. at Round 0-1) are blocked from target recognition by MTX in the following two rounds (i.e. at Round 1-2 and Round 2-3), where conditional T cell activity returns to background levels that are comparable to ‘Untransduced’ T cells. Among the three clones tested, P07_C04 and P02_D09 exhibit quick inhibition rates while the target lysis by P03_A01 requires two rounds of MTX to be fully inhibited (i.e. such that target cell killing returns to background levels). As a control, FIG. 9B also shows that the tool CAR T cell is not inhibited by MTX.

In the second scenario (testing the ability for an inactive CAR T cell containing an antibody having conditional affinity to be turned from “off” to “on”)(FIGS. 9C and 9D), conditional T cells that are incubated with MTX (i.e. at Round 0-1) are inhibited from lysing the target cells (see FIG. 9D, Round 1 data points), but this inhibition is relieved in subsequent rounds (Rounds 1-2 and Rounds 2-3) when the conditional T cells are incubated in co-cultures that did not contain MTX (see FIG. 9D, Round 2 and Round 3 data points). As a control, FIG. 9D also shows that the tool CAR T cell is not inhibited by MTX.

Thus, this example shows that conditional CAR T cells can be reversibly inhibited by MTX.

### Example 10: Short-Term Inhibition of EGFR-Specific Conditional CAR T Cell Activity

This example examines the cytotoxicity of conditional CAR T cells containing various different anti-EGFR antibodies having conditional affinity against EGFR-expressing target cells, in the presence and absence of methotrexate (MTX).

The anti-EGFR clones tested in this example were: P01_F02, P07_H08, P08_D06, P05_A11, and P15_F02 (each are described previously in, for example, Tables 5, 6, and 7). In addition, an EGFR-specific tool T cell was prepared, as were untransduced T cells.

The target cell line used in this assay was U87MG (obtained from American Type Culture Collection), which is a glioblastoma cell line that expresses EGFR. U87MG cells were transduced to stably express GFP and firefly luciferase, as in Example 4.

In vitro cytotoxicity assays were carried out in 96-well plates by co-culturing 50,000 T cells with 100,000 U87MG cells, for each different T cells described above. In addition, each of the co-cultures contained either 100 µM MTX or 0 µM MTX. In both conditions, 1 mM leucovorin was supplemented to circumvent MTX toxicity. Co-cultures were maintained in a final volume of 100 ul of RPMI 1640 medium for 48 hours at 37 C with 5% CO₂. Target cell killing was assessed and plotted as described in Example 4.

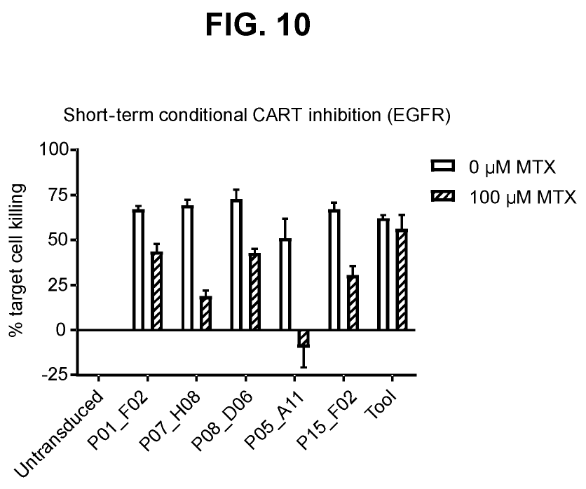

FIG. 10 shows the results for each EGFR-specific CAR, in the absence of MTX or the presence of 100 µM MTX. In the graph, the Y-axis shows the % target cell killing, and the X-axis shows the different EGFR-specific CAR T cell in the co-culture. In addition, for each different EGFR-specific CAR, two bars are present: left side: no MTX and right side: 100 µM MTX (also indicated in the figure legend). As shown in FIG. 10, all of the different clones of EGFR-specific conditional CAR T cell exhibited significantly reduced cytotoxic activity towards the target cells in the presence of MTX as compared to in the absence of MTX. In contrast, conventional EGFR-specific CAR T cells (Tool) were not affected by MTX in terms of their target lysis efficiency.

Thus, this experiment demonstrates that the cytotoxic activity CAR T cells which contain an anti-EGFR antibody having conditional affinity provided herein in the scFv of the CAR can be modulated by MTX.

### Example 11: Short-Term Inhibition of CD33-Specific Conditional CAR T Cell Activity Using MTX Analogues

This example examines the cytotoxicity of conditional CAR T cells containing various different anti-CD33 antibodies having conditional affinity against CD33-expressing target cells, in the presence of different methotrexate analogues that were modified at the glutamate tail of methotrexate. Included in this example as methotrexate analogues are: i) methotrexate that is covalently linked to a chain of 4 glutamate groups (“MTX-G4”)(Moravek Chemicals, CA) and ii) biotinylated methotrexate (“MTX-B”)(custom synthesized by Carbosynth, Berkshire, UK).

The anti-CD33 clones tested in this example were: P07_C04, P02_D09, P02_E08, and P03_H04. In addition, a tool T cell was prepared as described in Example 4, as were untransduced T cells. Also, each of the different conditional CAR T cells described above was generated in duplicate from PBMC cells from two different human donors.

The target cell line used in this assay was MV4-11 (obtained from American Type Culture Collection), which expresses CD33. MV4-11 cells were transduced to stably express GFP and firefly luciferase, as described in Example 4.

In vitro cytotoxicity assays were carried out in 96-well plates by co-culturing 50,000 T cells with 100,000 MV4-11 cells, for each different T cells described above. In addition, each of the co-cultures contained one of the following MTX-related conditions: 100 µM MTX, 0 µM MTX, 100 µM MTX-G4, or 100 µM MTX-B. In all conditions, 1 mM leucovorin was supplemented to circumvent MTX toxicity. Co-cultures were maintained in a final volume of 100 ul of RPMI 1640 medium for 48 hours at 37 C with 5% CO₂. Target cell killing was assessed and plotted as described in Example 4.

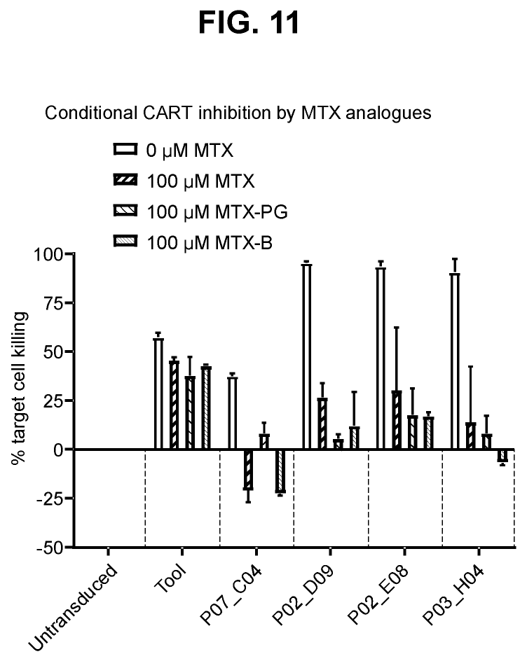

FIG. 11 shows the averaged results for each CD33-specific CAR, in the absence of MTX, or the presence of 100 µM MTX, MTX-G4, or MTX-B. In the graph, the Y-axis shows the % target cell killing, and the X-axis shows the different CD33-specific CAR T cell in the co-culture. In addition, for each different T cell, four bars are present, from left to right: 0 µM MTX, 100 µM MTX, 100 µM MTX-G4, and 100 µM MTX-B (also indicated in the figure legend). (No bars are present for the untransduced cells, as they had 0% target cell killing.) As shown in FIG. 11, all of the different clones of conditional CAR T cell exhibited significantly reduced cytotoxic activity towards the target cells in the presence of MTX, MTX-G4, or MTX-B, as compared to in the absence of MTX. In contrast, conventional CAR T cells (Tool) were insensitive to the presence of MTX, MTX-G4, or MTX-B in terms of their target lysis efficiency.

Thus, this experiment demonstrates that the cytotoxic activity CAR T cells which contain an anti-CD33 antibody having conditional affinity provided herein in the scFv of the CAR can be modulated by MTX or MTX covalently linked to a conjugate molecule, including polyglutamate (MTX-G4) and biotin (MTX-B).

Although the disclosed teachings have been described with reference to various applications, methods, kits, and compositions, it will be appreciated that various changes and modifications can be made without departing from the teachings herein and the claimed invention below. The foregoing examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

1. An antibody with conditional affinity which specifically binds an antigen with higher affinity under a low agent concentration than under a high agent concentration, wherein
a. the antibody comprises a heavy chain variable region (VH) and a light chain variable region (VL); and
b. the agent is a small molecule.

2. An antibody with conditional affinity which specifically binds an antigen with higher affinity under a high agent concentration than under a low agent concentration, wherein
a. the antibody comprises a heavy chain variable region (VH) and a light chain variable region (VL); and
b. the agent is a small molecule.

3. The antibody of claim 1, wherein the agent is methotrexate.

4. The antibody of claim 1, wherein the ratio of: [K_{D} of the antibody-antigen binding at 25° C. at high agent concentration] / [K_{D} of the antibody-antigen binding at 25° C. at low agent concentration] is more than, or ranges between 2, 3, 4, 8, 10, 16 or more.

5. The antibody of claim 1, wherein the binding of the antibody to the antigen under a low agent concentration at 25° C. has a K_{D} of about 1 nM to about 500 nM.

6. The antibody of claim 2, wherein the ratio of: [K_{D} of the antibody-antigen binding at 25° C. at low agent concentration] / [K_{D} of the antibody-antigen binding at 25° C. at high agent concentration] is more than, or ranges between 2, 3, 4, 8, 10, 16 or more.

7. The antibody of claim 1, wherein the binding of the antibody to the antigen under a high agent concentration at 25° C. has a K_{D} of about 1 nM to about 500 nM.

8. The antibody of claim 1, wherein the low agent concentration is about 0 nM to about 1 nM, and the high agent concentration is about 5 nM to about 100 mM.

9. The antibody of claim 1, wherein the low agent concentration is about 0 nM to about 1 µM, and the high agent concentration is about 5 µM to about 100 mM.

10. The antibody of claim 1, wherein the high agent concentration is at least about 2-times, 3-times, 5-times, 10-times, 20-times, 50-times, 100-times, or 1000-times greater than the low agent concentration.

11-12. (canceled)

13. The antibody of claim 1, wherein the VH region comprises a CDR1 and a CDR2 of the partial VH sequence shown in SEQ ID NO: 1.

14. The antibody of claim 1, wherein the VH region comprises a CDR1 comprising an amino acid sequence as shown in SEQ ID NO: 43, 116, or 117 and a CDR2 comprising an amino acid sequence as shown in SEQ ID NO: 44 or 118.

15. The antibody of claim 1, wherein the VH region comprises a framework region 3 (FR3) comprising the amino acid sequence Cys-Ala-Ala.

16. The antibody of claim 1, wherein the VH region comprises an amino acid sequence as shown in SEQ ID NO: 1.

17. The antibody of claim 1, wherein the small molecule agent specifically binds to the antibody at a location comprising the VH region.

18. A library comprising polynucleotides encoding a heterogeneous population of antibodies, wherein at least a first polynucleotide of the polynucleotides encodes a heavy chain variable region (VH) of an antibody that specifically binds an antigen with higher affinity under a low agent concentration than under a high agent concentration, wherein
a. the antibody comprises a VH and a light chain variable region (VL); and
b. the agent is a small molecule.

19-20. (canceled)

21. An isolated nucleic acid encoding at least the VH region, the VL region, or both the VH region and VL region of an antibody of claim 1 .

22. An isolated nucleic acid encoding a polypeptide comprising:
a single-chain Fv domain (scFv) which comprises a heavy chain variable region (VH) and a light chain variable region (VL), and which specifically binds an antigen with higher affinity under a low agent concentration than under a high agent concentration;
a transmembrane domain; and
an intracellular signaling domain,
wherein the agent is a small molecule.

23-25. (canceled)

26. An isolated cell line that produces the antibody of claim 1.

27. (canceled)

28. A host cell comprising a recombinant expression vector comprising the isolated nucleic acid of claim 21.

29. A method of producing an antibody with conditional affinity, the method comprising: culturing a host cell of claim 28 under conditions wherein the antibody is produced; and recovering the antibody.

30-31. (canceled)

32. A method of treating a disorder in a subject, the method comprising administering to a subject in need thereof an antibody of claim 1.

33-34. (canceled)
